(19) United States
(12) Patent Application Publication
(10) Pub. No.: US 2016/0174814 A1
(43) Pub. Date: Jun. 23, 2016

(54) ENDOSCOPE WITH SHARED WORKING CHANNEL

(71) Applicant: Morena Medical Applications Ltd., Tel-Aviv (IL)

(72) Inventor: Igor IGOV, Lod (IL)

(21) Appl. No.: 15/055,971

(22) Filed: Feb. 29, 2016

Related U.S. Application Data
(63) Continuation-in-part of application No. PCT/IL2014/050779, filed on Aug. 31, 2014.
Application No. PCT/IL2014/050779 is a continuation-in-part of application No. 14/492,297, filed on Sep. 22, 2014.
Application No. 14/492,297 is a continuation-in-part of application No. PCT/IL2014/050779, filed on Aug. 31, 2014.
(60) Provisional application No. 61/872,637, filed on Aug. 31, 2013.
(60) Provisional application No. 61/880,941, filed on Sep. 22, 2013.
(60) Provisional application No. 61/891,075, filed on Oct. 15, 2013.
(60) Provisional application No. 61/910,235, filed on Nov. 29, 2013.
(60) Provisional application No. 61/988,162, filed on May 3, 2014.
(60) Provisional application No. 62/015,271, filed on Jun. 20, 2014.

(51) Int. Cl. A61B 1/005; A61B 1/018; A61B 1/05; A61B 1/00

(52) U.S. Cl. CPC A61B 1/0051; A61B 1/00078; A61B 1/018; A61B 1/05

(57) ABSTRACT

Multiple tasks may be performed in a lumen of a living creature accessible via a path. One or more guides are optionally supplied. Optionally the guides communicate with a working channel of an endoscope. A tool may be conveyed along a guide through the working channel of the endoscope. For example a first tool may be conveyed to the lumen unblocking the working channel for a second tool. Optionally the second tool may subsequently be conveyed along a second guide. In some embodiments the guides may include thin channels in communication with the working channel via a longitudinal slit. Optionally the working channel may include a slit. For example large objects may be conveyed along the working channel partially protruding from the longitudinal slit.

### RELATED APPLICATIONS

This application is a Continuation-in-Part (CIP) of PCT Patent Application No. PCT/IL2014/050779 having International filing date of Aug. 31, 2014, which claims the benefit of priority under 35 USC §119(e) of U.S. Provisional Patent Application Nos. 61/872,637 filed on Aug. 31, 2013, 61/880,941 filed on Sep. 22, 2013, 61/891,075 filed on Oct. 15, 2013, 61/910,235 filed on Nov. 29, 2013, 61/988,162 filed on May 3, 2014 and 62/015,271 filed on Jun. 20, 2014.

This application is also a Continuation-in-Part (CIP) of U.S. patent application Ser. No. 14/492,297 filed on Sep. 22, 2014, which is a Continuation-in-Part (CIP) of PCT Patent Application No. PCT/IL2014/050779 having International filing date of Aug. 31, 2014.

The contents of the above applications are all incorporated by reference as if fully set forth herein in their entirety.

### FIELD AND BACKGROUND OF THE INVENTION

The present invention, in some embodiments thereof, relates to a system for controlling tools in a lumen of a living creature and, more particularly, but not exclusively, to a catheter for conveying one or more tools through a shared working channel.

U.S. Published Patent Application No. 20040092794 discloses that, “A steerable endoscopic sheath has a proximal end, a distal end and a working channel lumen disposed therein. A plurality of solid state light emitting devices such as light emitting diodes are positioned near the distal end of the sheath and are selectively energized to illuminate internal body tissues. An imaging device such as a photo diode or CCD array creates an image from light reflected from the tissue. The distal tip of the endoscopic sheath is selectively moveable with a deflection device that is insertable into the sheath. The deflection device includes a tip deflection mechanism that allows a user to move the tip of the deflection device. With the deflection device inserted in the sheath, movement of the distal tip causes a corresponding movement in the distal tip of the sheath. The distal tip of the sheath has a shape retaining mechanism that allows it to retain the shape imparted by the deflection device once the deflection device is removed from the sheath.”

U.S. Pat. No. 8,398,540 discloses “A disposable endoscope body having an enclosure in its distal end for a non-disposable element, such as a video camera head. Because of its complexity and sensitivity, the camera head is non-disposable and cannot be sterilized. The camera head is sealed in the endoscope body, such that it does not contaminate the patient during the procedure. Sealing is achieved using an end cap. After use, the camera is withdrawn from the proximal end of the endoscope, which, being near to or within the workstation, has not been inserted into the subject and should be free of patient contamination. Passage of the camera out through the proximal end therefore avoids contamination by the patient, such that it can be used again without need for sterilization. A dispensing kit is described, for supplying the sterile endoscope body and for installing and sealing the camera with a minimum of manual intervention.”

U.S. Published Patent Application No. 2012/0035416 “relates generally to medical devices and methods. The present medical devices comprises a platform comprising a magnetically-attractive material, and a camera coupled to the platform and configured to be moved in at least three degrees of freedom relative to the platform, where the camera's movement in each respective degree of freedom is controlled by a separate actuator coupled to the platform. The medical devices further comprise a housing disposed around at least a portion of the camera, the housing being at least partially transparent, and a wiper arm configured to move relative to the housing. Some embodiments of the present multi-degree-of-freedom cameras for a medical procedure, comprises a platform comprising a magnetically-attractive material, an apparatus to moving the platform within a body cavity of a patient when the apparatus is outside the body cavity, the apparatus comprising a magnetic assembly, and a camera coupled to the platform, and configured to be moved in at least three degrees of freedom relative to the platform, where the camera's movement in each respective degree of freedom is controlled by a separate actuator coupled to the platform.”

U.S. Pat. No. 5,166,787 discloses an endoscope having a video device arranged at the distal end of the endoscope shaft. The video device is connected by means of a transmission system to a supply unit arranged at the proximal end of the endoscope shaft. The video device is provided with a lens for imaging an object field and an illumination unit. The lens and the image recorder are combined into a video unit which is held in such a movable manner at the endoscope shaft that the outer contour of the cross-section of the video unit lays essentially within the outer contour of the cross-section of the distal end of the endoscope shaft when being introduced into the cavity to be examined. After termination of the introduction procedure, the video unit can be moved in relation to the distal end of the endoscopic shaft beyond the outer contour of the cross-section and/or longitudinal section.

U.S. Pat. No. 7,431,694 discloses a guide system for use with an endoscope, and a method of use. The guide system can include a track, in the form of a rail, and a mating member for engaging the rail. The guide system can also include an accessory, such as an accessory guide tube through which a medical instrument can be carried external of the endoscope. An end cap can be provided to support the track relative to the distal end of the endoscope.

Additional background art includes U.S. Pat. No. 5,329,887.

### SUMMARY OF THE INVENTION

According to an aspect of some embodiments of the present invention there is provided a catheter for transporting material between outside a living creature and a lumen inside the living creature including: a working channel including a proximal opening for access from outside of the living creature and a distal opening for positioning inside a lumen of the living creature; and a plurality of elongated guides extending along at least a portion of a length of the working channel, each of the guides in communication with the working channel along at least 5 cm of a length thereof.

According to some embodiments of the invention, the material includes a tool transportable between a first location outside the proximal opening, a second location inside the working channel and a third location outside the distal opening further including: a carriage mating to at least one guide of the plurality of elongated guides; the carriage connected to the tool off center of a cross section of the tool, traveling along the at least one guide from a proximal position to a distal position thereby conveying the tool from the second location to the third location and rotating around an axis of the at least one guide in the distal position thereby moving the tool to a fourth location away from an axis of the working channel.

According to some embodiments of the invention, the tool includes a camera.

According to some embodiments of the invention, in the third location a lens of the camera clears an outer cross section of the catheter.

According to some embodiments of the invention, the catheter further includes a mount external to the working channel, the mount rigidly supporting the camera outside the working channel.

According to some embodiments of the invention, in the tool includes a lens and at the third location the lens clears an outer cross section of the catheter.

According to some embodiments of the invention, the catheter further comprises: a closed channel of length at least 50% of the sleeve, the closed channel having a proximal opening for access from outside of the living creature and the closed channel closed from the region; a steering mechanism the steering mechanism reversibly inserted into the closed channel for steering the catheter.

According to some embodiments of the invention, the catheter further comprises: an engagable fitting for inhibiting rotation of the steering mechanism with respect to the closed channel.

According to some embodiments of the invention, the channel and the steering mechanism have matching non-rotationally symmetric cross sections such that the steering mechanism is inhibited from rotating in the channel.

According to some embodiments of the invention, the steering mechanism fits into the channel in a single orientation.

According to some embodiments of the invention, the closed channel is sealed off from the region.

According to some embodiments of the invention, the catheter further comprises: a working channel including a proximal opening for access from outside of the living creature and a distal opening for positioning inside a region of the living creature the working channel for transporting a tool between a first location outside the proximal opening, a second location inside the working channel and a third location outside the distal opening.

According to some embodiments of the invention, the closed channel is sealed off from the working channel.

According to some embodiments of the invention, the catheter further comprises: a closed channel having a proximal opening for access from outside of the living creature and closed from the region; a steering mechanism the steering mechanism reversibly inserted into the closed channel for steering the catheter.

a steering mechanism said steering mechanism reversibly inserted into said closed channel for steering said catheter.

According to some embodiments of the invention, the catheter further includes a source of suction connected to the proximal opening for suctioning a fluid from the lumen through the working channel subsequent to the moving.

According to some embodiments of the invention, at the at least one guide includes a guide channel having a circular cross section at least at a distal portion thereof.

According to some embodiments of the invention, the carriage is at least as long as the working channel and the rotating is by twisting a proximal portion of the carriage.

According to some embodiments of the invention, the communication extends to the distal opening.

According to some embodiments of the invention, the communication extends to the distal opening.

According to some embodiments of the invention, at least one of the guides includes a guide channel.

According to some embodiments of the invention, the guide channel includes a longitudinal opening communicating between the guide channel and the working channel.

According to some embodiments of the invention, the longitudinal opening has a width of between 1/10 and ¼ the width of the guide channel.

According to some embodiments of the invention, the catheter further includes a longitudinal opening communicating between the working channel and the lumen.

According to some embodiments of the invention, the catheter further includes a stiffener holding the longitudinal opening open.

According to some embodiments of the invention, the catheter further includes a mount for supporting the tool on an outer surface of a distal portion of the catheter.

According to some embodiments of the invention, the catheter further includes a mount external to the working channel, the mount rigidly supporting the tool outside the working channel.

According to some embodiments of the invention, the mount supports the tool substantially out of a contour of a cross section of the working channel.

According to some embodiments of the invention, a cross sectional area of each guide of the plurality of guides is less than one fifth of a cross sectional area of the working channel.

According to some embodiments of the invention, the catheter further includes a first distal finger for directing a distal end of a first guide of the elongated guides; and a control system accessible from a proximal end of the catheter for directing the first finger relative to an axis of the catheter.

According to some embodiments of the invention, the catheter further includes a second distal finger for directing a distal end of a second guide of the elongated guides; and wherein the control system is further for directing the second finger relative to an axis of the catheter.

According to some embodiments of the invention, the working channel has a minimum cross sectional area of at least one ninth the minimum cross sectional area of the catheter between the distal opening and the proximal opening.

According to some embodiments of the invention, each of the guides extends to the distal opening.

According to some embodiments of the invention, each of the guides extends to the distal proximal opening.

According to an aspect of some embodiments of the present invention there is provided a catheter including: a working channel between a proximal opening for access from outside of a living creature and a distal opening for positioning inside a lumen of the living creature the working channel having a minimum cross sectional area of at least one ninth the minimum cross sectional area of the catheter between the distal and proximal openings; and a longitudinal opening along at least a portion of a length of the working channel the longitudinal opening in communication with an exterior of the catheter the longitudinal opening having at least a length of 10 cm.

According to some embodiments of the invention, the catheter further includes a tool having a cross section greater than a cross section of the working channel in at least one dimension, the tool configured for passing along the working channel between the distal opening and the proximal opening while at least partially protruding through the longitudinal opening during the passing.

According to some embodiments of the invention, the catheter further includes an elongated guide extending along at least a portion of a length of the working channel communicating with an interior of the working channel.

According to some embodiments of the invention, the catheter further includes a carriage connected to the tool the carriage mating to the elongated guide for traveling along the guide from a proximal position to a distal position thereby conveying the tool along at least a portion of a length of the working channel.

According to some embodiments of the invention, the longitudinal opening is biased in an open configuration.

According to some embodiments of the invention, the catheter further includes a stiffener for holding the longitudinal channel open.

According to some embodiments of the invention, the longitudinal opening extends from the proximal opening to the distal opening.

According to an aspect of some embodiments of the present invention there is provided a system for transporting material along a path between outside a living creature and a region inside the living creature including: a plurality of elongated guides extending along the path in communication with the path; a tool transportable between a first location outside the living creature, a second location inside the path and a third location inside the region; and a carriage mating to at least one guide of the plurality of elongated guides, the carriage traveling along the at least one guide from a proximal position to a distal position thereby conveying the tool from the second location to the third location.

According to an aspect of some embodiments of the present invention there is provided a catheter including: a plurality of channels each channel including a proximal opening for access from outside of a living creature and a distal opening for positioning inside a lumen of the living creature; a plurality of fingers, each the finger including a distal opening of a channel of the plurality of channels; and a control mechanism at a proximal side of the catheter to direct at least two of the fingers in different directions.

According to some embodiments of the invention, the catheter further includes a working channel with a cross sectional area at least four times the cross sectional area of each channel of the plurality of channels and wherein the each channel of the plurality of channels communicates with the working channel along a length of the working channel and wherein the fingers are located around a distal opening of the working channel.

According to an aspect of some embodiments of the present invention there is provided a method for performing one or more tasks inside a lumen of a living creature, the lumen accessible through a working channel of a catheter the working channel including a distal opening inside the lumen and proximal opening outside the lumen including: providing one or more elongated guides located along at least a portion of a length of the working channel, each of the one or more guides in communication with the working channel along the at least a portion of the length; inserting a first tool into the proximal opening to a location in the working channel; blocking at least 50% of a cross section of the working channel at the location with the first tool; and unblocking the working channel by conveying the first tool along a first guide of the one or more guides from the location to a site in the lumen.

According to some embodiments of the invention, the working channel and a closed channel with a proximal opening are included in a sleeve, the and the method further comprising: inserting a steering mechanism into the proximal opening; introducing a distal portion of the sleeve into the region while steering the distal portion with the steering mechanism; and retrieving the steering mechanism through the proximal opening and retrieving said steering mechanism through said proximal opening.

According to some embodiments of the invention, the method further includes conveying a second tool along the working channel to a second site via a second guide of the one or more guides, the conveying while the first tool is located at the site.

According to some embodiments of the invention, the method further includes supporting the first tool an outer surface of the catheter.

According to some embodiments of the invention, the method further includes transporting a fluid along the working channel between the distal opening and the proximal opening subsequent to the unblocking while the first tool is located at the first site.

According to some embodiments of the invention, the method further includes retrieving the first tool out from the living creature through the proximal opening.

According to some embodiments of the invention, the method further includes conveying a second tool along the working channel to the lumen via the first guide subsequent to the retrieving.

According to some embodiments of the invention, the method further includes conveying the first tool along the working channel to a second site in the region via a second guide of the one or more guides, subsequent to the retrieving.

According to some embodiments of the invention, the method further includes suctioning a fluid from the lumen through the working channel subsequent to the unblocking while the first tool is in the lumen.

According to some embodiments of the invention, the first tool includes a camera and the method further includes: viewing the lumen via the camera during the suctioning.

According to some embodiments of the invention, the fluid at least 50% of a cross section of the working channel over at least 50% of a length of the working channel is open for transport of the fluid during the suctioning.

According to an aspect of some embodiments of the present invention there is provided a method for performing one or more tasks inside a region of a living creature, the region accessible by a path including: providing one or more elongated guides located along at least a portion of a length of the path, each of the one or more guides in communication with the path along the at least a portion of the length; inserting a first tool to a location on the path; blocking at least 50% of a cross section of the path at the location with the first tool; and unblocking the path by conveying the first tool along a first guide of the one or more guides to a site in the region.

According to an aspect of some embodiments of the present invention there is provided a method for performing one or more tasks inside a lumen of a living creature, the lumen accessible through a working channel of a catheter the working channel including a distal opening inside the lumen and proximal opening outside the lumen including: providing one or more elongated guides located along a length of the working channel, each of the one or more guides in communication with the working channel along the length each of the one or more guides having a proximal opening and a distal opening and providing first carriage connected to the tool off center of a cross section of the tool, the carriage mating to a first guide of the elongated guides the first carriage having a length greater than the working channel; blocking at least 50% of a cross section of the working channel at a location with the first tool; conveying the first tool along the working channel and out the distal opening of the working channel by means of the guide; and unblocking the working channel by twisting a proximal end of the guide to move the tool away from an axis of the working channel.

According to some embodiments of the invention, the method further includes performing a second function with the working channel subsequent to the unblocking.

According to some embodiments of the invention, the first tool includes a camera and the unblocking further includes moving a lens of the camera to clear an outer cross section of the catheter.

According to some embodiments of the invention, the method further includes rigidly supporting the camera on the catheter on the catheter away from the working channel.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart illustrating a method of controlling a tool in a lumen of a living creature in accordance with some embodiments of the current invention;

FIG. 2 is a flowchart illustrating sharing of a working channel in accordance with some embodiments of the current invention;

FIG. 3 is a chart of states of a tool and shared channel in accordance with an embodiment of the present invention;

FIGS. 4A, A′, B, B′ are a block diagrams illustrating systems for controlling a tool in a lumen in accordance with some embodiments of the current invention;

FIGS. 5A, B are two perspective views of a tool including a camera and a carriage in accordance with an embodiment of the current invention;

FIGS. 6A-G are perspective drawings illustrating deploying of a tool including camera in accordance with an embodiment of the current invention;

FIGS. 7A-C are perspective drawings illustrating the deploying of a pincers tool along with a camera using a shared channel in accordance with an embodiment of the current invention;

FIG. 8 is a perspective drawing illustrating a camera tool, an outer sleeve and an inner endoprobe in accordance with some embodiments of the current invention;

FIG. 9 is a perspective drawing illustrating a camera tool and an endoprobe including a pincer tool sharing a working channel of an outer sleeve in accordance with some embodiments of the current invention;

FIG. 10 is a perspective end on view of an endoprobe inserted into an outer sleeve in accordance with some embodiments of the current invention;

FIGS. 11A-E are perspective views illustrating alternative options for deploying a camera tool according to some embodiments of the current invention;

FIGS. 12A-D are perspective views of insertion of an active inner probe into a passive outer sleeve in accordance with some embodiments of the current invention;

FIGS. 13A-C are perspective view illustrations of an integrally steered outer sleeve catheter in accordance with some embodiments of the current invention;

FIG. 14 is a perspective view of an outer sleeve in accordance with some embodiments of the current invention;

FIGS. 15A, B are a perspective views of a working channel being shared by a suction tool and a flushing flow in accordance with some embodiments of the current invention;

FIGS. 16A-C illustrate use of guide channels for independent instruments in accordance with some embodiments of the current invention;

FIGS. 17A-E illustrate deployment and retrieval of a sample bag via a working channel in accordance with some embodiments of the current invention;

FIG. 18 is a flow chart of a method of employing a foldable tool in accordance with an embodiment of the current invention;

FIG. 19 is a state diagram of a foldable tool in accordance with an embodiment of the current invention;

FIGS. 20A, B are schematic illustrations of a working channel with tracks in accordance with some embodiments of the current invention;

FIG. 21 illustrates an endoprobe with external guide channels in accordance with some embodiments of the current invention;

FIG. 22 illustrates an endoprobe with external guide tracks inserted into a working channel in accordance with some embodiments of the current invention;

FIG. 23 is a perspective view of a tool supported on multiple guides in accordance with an embodiment of the present invention;

FIGS. 24A, B are a perspective views of rounded guides tracks in accordance with an embodiment of the present invention;

FIG. 25 is a perspective view of T-shaped slot guides in accordance with an embodiment of the present invention;

FIGS. 26A-C are perspective views of a catheter in use for removing a growth (for example a polyp) in accordance with an embodiment of the current invention;

FIGS. 27A, B are a perspective illustrations of a catheter including stiffening rings in accordance with an embodiment of the current invention;

FIG. 28 is a perspective view of cameras at multiple viewing positions in accordance with an embodiment of the present invention;

FIGS. 29A-C are perspective illustrations of a camera deployed ahead of a catheter in accordance with an embodiment of the present invention;

FIGS. 30A-G illustrate a large sample bag mounted on an external guide of a catheter in accordance with an embodiment of the current invention;

FIGS. 31A-F illustrate various options for a cross section of a multi-function catheter in accordance with some embodiments of the current invention;

FIGS. 32A-C are perspective views of a catheter with distal fingers in accordance with an embodiment of the present invention;

FIG. 33 is an exploded view of a catheter and a single distal finger in accordance with an embodiment of the present invention;

FIG. 34 illustrates a distal finger fitting a distal slit in accordance with an embodiment of the present invention;

FIGS. 35A-D illustrate frames of a catheter in accordance with some embodiment of the present invention;

FIGS. 36A, B illustrate a magnetic actuator in accordance with an embodiment of the present invention;

FIG. 36C illustrates a frame steered by magnetic actuators in accordance with an embodiment of the present invention;

FIGS. 37A-C illustrate a joint immobilizer for a catheter in accordance with an embodiment of the current invention;

FIGS. 38A-B are perspective distal and proximal views of a sleeve with a closed channel in accordance with an embodiment of the present invention;

FIGS. 39A-B are a perspective proximal views of a removable steering mechanism in accordance with an embodiment of the present invention;

FIG. 40 is a flowchart illustration of a method of steering a sleeve in a lumen in accordance with an embodiment of the current invention;

FIG. 41A is a perspective proximal view of a multi-channel sleeve in accordance with an embodiment of the present invention;

FIG. 41B is a perspective distal view of a sleeve with exemplary tools in accordance with an embodiment of the present invention;

FIGS. 42A-B are a perspective distal view of a sleeve with exemplary tools in accordance with an embodiment of the present invention; and

FIGS. 43A-B are a perspective view illustrations of a multi-camera endoscope in accordance with an embodiment of the current invention.

### DESCRIPTION OF SPECIFIC EMBODIMENTS OF THE INVENTION

The present invention, in some embodiments thereof, relates to a system for controlling tools in a lumen of a living creature and, more particularly, but not exclusively, to a catheter for conveying one or more tools through a shared working channel.

Overview

Catheter with Shared Working Channel

An aspect of some embodiments of the present invention relates to using a catheter to access a lumen inside a living creature. Optionally the catheter includes a shared working channel and/or one or more narrower guides (guides may include for example one or more additional channels). Each guide may communicate with the working channel over all or some of the length thereof. An operator employs, a guide to convey a first tool through the working channel to the lumen and/or to deploy the first tool in the lumen. Optionally deploying the tool into the lumen unblocks the working channel. Subsequently the operator may use a second guide to convey a second tool through the working channel to the lumen. Optionally one guide may be used to manipulate one tool in the lumen while the working channel and/or another guide is used to transport another tool through the working channel to the lumen. Optionally, operator employs the working channel and/or guides to control (for example to support and/or manipulate) a tool outside a distal opening of the working channel in the lumen. Optionally, the operator may remove a tool and/or reinsert the tool after removal (for example on the same guide and/or on a different guide). Optionally the lumen may also be used for transport of fluids, for example for suctioning fluids and/or debris from the lumen, for washing objects in the lumen, for irrigating the lumen, and/or for applying medicinal chemicals. For example, a camera and/or a tool may be retrieved and/or redeployed to a different position (for example to get a better view of an object) without moving and/or removing the catheter.

In some embodiments, a tool may be controlled inside a lumen. For example, a camera and/or a tool may be deployed in the lumen away from the axis of a catheter. For example, a camera may be deployed in a location that allows a viewing forward, backwards and/or to the side. For example, an operator may control a tool in the lumen by twisting a proximal end of a carriage connected to a guide and/or the tool. For example the operator may manipulate a tool in the lumen, unblock the lumen and/or unblock the distal opening of the lumen. The unblocked lumen may be used for example for passing liquid through the working channel (for example for irrigating and/or suctioning the lumen). For example, a camera may be deployed in a lumen through the working channel and/or deployed away from the working channel. Subsequently, while the viewing the lumen with the camera, the large working channel is optionally used to suction debris from the lumen and/or for an operative procedure.

Controlling One or More Tasks in Lumen with a Narrow Access Path

An aspect of some embodiments of the present invention relates to controlling one or more tasks inside a lumen accessible via a path and/or catheter channel. In some embodiments, one or more narrow guides may be placed along the path. For example, an operator located at the proximal end of the guides may use the guides and/or carriages attached to the guides to deploy and/or manipulate multiple tools in the lumen. Optionally, the guide may remain in position and/or substantially stationary while the tool is conveyed along the guide and/or stabilized by the guide and/or manipulated using the guide.

In some embodiments, a tool may be mountable external to the catheter. For example an external surface of a catheter may include a mount for connecting to a tool. Alternatively or additionally, a tool may include a mount for connecting to an outside surface of the catheter. For example, the tool may be conveyed by the guide through the working channel to the lumen. Outside the working channel (for example in the lumen) the tool may be supported using the guide and/or manipulated using the guide and/or directed using the guide. For example the tool may be directed to a location for mounting to the external surface of the catheter. Alternatively or additionally, a tool may be connected to an object outside of the catheter. Optionally, the tool is returned back to the working channel and/or the guide.

In some embodiments a guide may extend along the entire path. For example the guide may extend from a proximal opening to a distal opening of the path and/or channel. For example a proximal end of a guide may be accessible from outside the patient and/or a distal end of the guide may be exposed to the lumen. Alternatively or additionally, a guide and/or a catheter may be place along a portion of an access path to the lumen. For example, the proximal end of the guide may not reach all the way out of the patient and/or the distal end of the guide may not reach the lumen. For example the guide may be placed at a along a narrowing portion of the access path. Optionally, all or part of the guide may be retrieved from the patient after use. Alternatively or additionally, a guide may remain in a patient.

Working Channel with a Longitudinal Opening

An aspect of some embodiments of the current invention relates to a system and/or method for conveying a large object inside of a living creature. In some embodiments, a catheter may be supplied with a working channel having a longitudinal opening. For example, a working channel may have an opening and/or slit along all or part of its length. The slit may allow conveying a large object along the channel and/or positioning the channel over an endoprobe. In some embodiments, a catheter including a slit working channel may include no other channels; one or more other slit working channel; one or more closed cross section working channel; one or more guides in communication with the working channel; and/or one or more guides in communication with a space external to the catheter. For example the large object may be transported partially inside a channel and partially protruding from the opening and/or slit in the channel.

In some embodiments, while transporting an object in a lumen with a longitudinal opening the portion of the volume of the object inside the channel may range for example from 10% to 50% and or from 50% to 75% and/or from 75% to 90% and/or from 90% to 100%. The rest of the object may protrude through the longitudinal opening. Optionally the object may be enclosed in a vessel (for example a bag) while being transported. For the sake of the current disclosure, the term inside when applied to an elongated channel having a longitudinal opening will mean the space in the cross section enclosed by the walls of the catheter and line joining the edges of the opening and/or openings.

In some embodiments a sleeve or tube may be inserted into a slit working channel. For example a sleeve and/or a carriage may close the slit of the channel over all and/or part of its length. A sleeve may optionally have a closed geometry (for example encircling all or part of the interior of the working channel). Alternatively or additionally, a sleeve may have an open geometry, for example close the slit of the working channel without enclosing any space. Alternatively or additionally, a sleeve may have a closed geometry over one portion of its length and/or an open geometry over another portion of its length. For example, when the slit is closed, the channel may be used for transporting fluid and/or isolating a transported object (for example to avoid contamination).

In some embodiments a longitudinal guide may be supplied communication with an external surface of a catheter. Optionally a large object may be transported along the guide inside of a living creature.

In some embodiments, a connecting channel may unite two spaces. For example a connecting channel may unite a working channel and an outside surface of a catheter. Alternatively or additionally, a connecting channel may unite two working channels in a catheter. For example a connecting channel may run longitudinally along all or part of a working channel. Optionally, a carriage inserted into the connecting channel may partially and/or completely block communication between the two spaces. For example the carriage may be used to control communication between the two spaces. Alternatively or additionally, the connecting channel may include a guide channel for conveying and/or controlling a tool. For example a connecting guide channel may be used to convey a tool in either or both of the two spaces. For example, a connecting guide channel connecting two working channels may be used to convey a tool to a lumen and/or deploy the tool to the lumen from a first working channel. The same connecting guide may then be used to retrieve the tool through the second working channel. For example, a connecting guide channel connecting a working channel to an external surface may be used to convey a tool to a lumen and/or deploy the tool to the lumen from a first working channel. The same connecting guide may then be used to retrieve the tool along the outside of the catheter. Optionally the cross section of a slit, guide and/or working channel may take on one or more of many shapes, oval, rectangular, T-shaped, triangular, circular etc. For example the tool may be moved with respect to the axis of the guide and/or between lumens by twisting a carriage connected and/or inserted in the guide.

Irrigation and/or Extraction

An aspect of some embodiments of the current invention relates to system and or method for transporting materials into or out of a living creature. For example transporting material may include irrigating and/or draining and/or flushing and/or cleaning a target inside a living creature. For example a working channel of a catheter may be used to extract fluid and/or a solid object and/or a high viscous material from a lumen. Optionally, simultaneous to extraction, a tube and/or another channel may be provided to irrigate the lumen. For example the tube and/or other channel may be connected to the same catheter that is used for the extraction. In some embodiments, a catheter may be used to insert and/or retrieve tools and/or support and/or manipulate a tool inside the living creature before and/or during and/or after material transport. For example a catheter may be used for sequential and/or simultaneous transport of material, conveying of a tool and/or control of a tools. Optionally, a catheter may perform various transport and/or conveying functions without moving and/or removing the catheter.

### Introducing Multiple Tools Through Narrow Working Channel

An aspect of some embodiments of the present invention relates to introducing and/or retrieving multiple tools through a narrow working channel. Optionally a working channel may include the working channel of a catheter which is positioned along the path to a treatment site. In some embodiments, one or more tools are optionally passed sequentially through the working channel into the lumen of a patient. In some embodiments, one or more guides may be used to convey one or more tools through the working channel and/or to unblock the working channel. Optionally one tool may be passed into the working channel after the previous tool has passed out of the working channel. Alternatively or additionally, multiple tools may be passed through the working channel in a line. For example, all of the tools may be attached to a single guide. Alternatively or additionally, one or more guides may be used to convey each of a plurality of tools independently through the working channel. For example, the working channel may be too narrow for two tools to pass simultaneously. Optionally, a first tool may be directed along a guide from a location in the working channel to a site outside the working channel unblocking the working channel. Subsequently, the unblocked working channel may be used for another function. For example the unblocked working channel may be used for transporting a fluid into or out of the lumen. Alternatively or additionally, a second tool may be passed along a second guide through the unblocked working channel and/or past the location vacated by the first tool.

In some embodiments, the guide may be used to return a tool from the lumen to outside the lumen, for example to the working channel and/or to return the tool to outside the body of the living creature. For example, a tool may be deployed into a lumen and/or later returned to the working channel and/or retrieved. The tool optionally will remain connected to the guide while it is deployed. Alternatively, the tool may be disconnected from the guide while it is deployed and/or reconnected to the guide when it is returned. For example, a tool may be connected to a tether for return and/or retrieval. Optionally the tool may be deployed returned and/or retrieved along a single guide. Alternatively or additionally multiple guides may be used for conveying, deploying, returning and/or retrieving a tool. For example, multiple guides may be used sequentially and/or simultaneously.

### Steering for Multi-Function Catheter

An aspect of some embodiments of the present invention relates to steering a multifunctional catheter. In some embodiments, a multifunctional catheter is steered through a path into a lumen. A steering assembly is optionally an integral part of the multifunction catheter. Alternatively or additionally, the steering assembly may include a separate probe and/or endoprobe. For example, a steering probe may be inserted into one or more channels of the catheter and/or used to steer the distal end of the catheter along a path to a target lumen. Once the catheter is in place, the steering assembly may be removed from the catheter and/or the channels may be freed for other tasks.

In some embodiments a steering mechanism may be inserted into a closed channel. For example the steering mechanism may be shielded from contact with the patient, a working channel and/or another tool. Optionally the steering mechanism may fit snugly and/or irrotationally into the channel.

### Folding Tool

An aspect of some embodiments of the current invention relates to a tool that includes a folded and an unfolded state. For example the tool may be transported in the folded state past a narrowing of an access channel. Optionally the tool is unfolded before use. The tool may optionally be retrieved in a folded and/or unfolded state.

### Distal Fingers for an Endoprobe

An aspect of some embodiments of the current invention relates to an endoprobe with a distal end inserted into a lumen of a living creature including one or more distal fingers. Optionally a finger may be controlled independently of other fingers. Alternatively or additionally, multiple fingers may be controlled in a synchronized manner. For example, a finger may be directed inward towards an axis of the endoprobe and/or outward away from the axis of the endoprobe. Optionally one or more fingers may be used to direct a tool. For example the fingers may be used to point and/or direct and/or support a tool located in a lumen of a living creature. Optionally a finger may be used to facilitate steering of the endoprobe. Optionally a finger may assist untangling a tool and/or the endoprobe. Optionally a finger may include a guide. Optionally a continuous guide may pass through the main body of the catheter to the proximal end of the finger and along the finger to the distal end of the finger. For example a tool and/or a material may be conveyed along a guide in the body of the endoscope to the finger. Optionally the tool and/or material may continue along a guide of the finger to a site in the living creature. Optionally the guide of the finger may include a channel. Optionally the guide and/or channel of the finger may be used to control a tool and/or transfer material to a location inside a living creature.

### Optional Features

In some embodiments, the working channel may include a distal opening and/or a proximal opening. Optionally, in operation the proximal opening is accessible from the outside the living creature and/or the distal opening is exposed to the lumen. For example, the working channel may be wide enough for passing a tool and/or a fluid through the proximal opening, along the length of the working channel to the distal opening and/or into the lumen. Each guide may include, for example, a narrow channel and/or a track running along all and/or part of the length of the working channel. Optionally a guide channel and/or a working channel may have a circular cross section. Alternatively or additionally a guide channel and/or a working channel may have a non-circular cross section (for example semi-circular, rectangular, triangular, etc.).

In some embodiments, a catheter may include one or more working channels. Optionally a working channel has a closed perimeter. Alternatively or additionally, a working channel may be open over a portion of its length. In some embodiments, the working channel may be located in the center of the catheter. Alternatively or additionally the working channel may be located off center. In some embodiments, a catheter may include multiple working channels and/or guides.

For the sake of the disclosure, unless specified otherwise, the term catheter may include various kinds of catheters and/or endoscopes for example an encephaloscope, a laryngoscope, an esophagoscope, a thoracoscope, an angioscope, a nephroscope, a colonoscope, a proctoscope, an arthroscope, a rhinoscope, an esophagoscope, a bronchoscope, a mediastinoscope, a gastroscope, a laparoscope, an amnioscope, a cystoscope, and/or a hysteroscope, a urinary catheter, a nephritic catheter, an abdominal catheter, a venous catheter, an arterial catheter, an intracranial catheter, an epidural catheter, a tracheal tube, a central venous catheter, a Swan-Ganz catheter, en embryo transfer catheter, an umbilical line, a Tuohy-Borst adapter, an intrauterine catheter and/or a Quinton catheter. The size (for example the cross sectional area and/or the length) and/or form of the catheter may optionally vary. For example the size and/or form of a catheter may vary according to the type of catheter. For example, a colonoscope, may have a length ranging between 130 to 250 cm and/or longer and/or have a working channel cross sectional area ranging between 3 to 5 mm² and/or between 5 to 10 mm² and/or between 10 to 15 mm² and/or between 15 to 25 mm² and/or have a guide channel cross sectional area ranging between 0.1 to 1 mm² and/or between 1 to 10 mm² and/or between 10 to 15 mm² and/or between 15 to 50 mm². A urinary catheter may for example have a length ranging between 20 to 25 cm and/or 25 to 40 cm and/or have a cross sectional area of a working channel ranging between 1 to 4 mm² and/or between 4 to 20 mm² and/or between 20 to 40 mm² and/or between 40 to 60 mm² and/or have a cross sectional area of a guide channel ranging between 0.1 to 1 mm², and/or between 1 to 5 mm², and/or between 5 to 30 mm² For the sake of the current disclosure, the term distal portion of a catheter may mean for example the most distal half of the catheter and/or the most distal quarter of the catheter and/or the most distal ⅛ of the catheter and/or the most distal 1/10 of the catheter and/or the most distal portion of the catheter of length ranging between 1 and 10 cm and/or ranging between 1 mm and 1 cm.

For the sake of the current disclosure, the proximal portion of a catheter may be defined for example as the most proximal half of the catheter and/or the most proximal quarter of the catheter and/or the most proximal ⅛ of the catheter and/or the most proximal 1/10 of the catheter and/or the most proximal portion of the catheter of length ranging between 1 and 10 cm and/or ranging between 1 mm and 1 cm. In some embodiments, the cross sectional area of a working channel may range between 90% to ½ the cross area of the catheter and/or between ½ and ¼ the cross area of the catheter and/or between ¼ and 1/9 the cross area of the catheter and/or between 1/9 and 1/25 the cross area of the catheter and/or between 1/25 and 1/100 the cross area of the catheter. Unless stated otherwise, as used herein, the ratio of cross section of a working channel to the cross section of the catheter refers to ratio of the minimal cross section of the channel between a proximal and a distal opening of the channel to the minimal cross sectional area of the catheter between the same openings. Alternatively or additionally, the ratio of cross sectional area of the working channel to the cross sectional area of the catheter may refer to the minimal local ratio of the cross sectional areas at any location between a proximal opening of the channel and a distal opening of the channel. Alternatively, the ratio of cross section of a working channel to the cross section of the catheter may refer to ratio of the maximum cross section of the channel between a proximal and a distal opening of the channel to the maximum cross sectional area of the catheter between the same openings. Alternatively or additionally, the ratio of cross sectional area of the working channel to the cross sectional area of the catheter may refer to the maximum local ratio of the cross sectional areas at any location between a proximal opening of the channel and a distal opening of the channel.

In some embodiments one or more guides may run along an inner wall of the working channel. Optionally the cross section of the working channel will be greater than the cross section of a guide associated with the working channel. For example a catheter and/or sleeve may include 1 guide and/or 2 guides and/or 3 guides and/or 4 guides and/or 5 guides and/or 6 guides and/or 7 to 10 guides and/or 10 to 15 guides. Alternatively, one or more guides may run along an outer surface of the catheter. In some embodiments, a guide may extend beyond the catheter body. For example a guide may run along the length of a working channel and/or extend out an opening of the working channel. Alternatively or additionally, a carriage may be connected to a guide. The tool may be connected to the carriage. The carriage may extend beyond the guide, for example, the guide may be inside the working channel of the catheter while the carriage and/or the tool may extend out of the working channel. For example the distance that a tool may extend beyond the distal end of the working may range over many values and/or depend on the kind of catheter. For example a tool may extend out from an endoscope a distance of less than one outer width of the endoscope and/or between one to five times the outer width of the endoscope and/or between 5 to 20 times the outer width of the endoscope and/or more. A carriage is optionally controlled by a physical manipulation by a user (for example a carriage may have a handle for manipulation by a user outside the lumen and/or a connection to the carriage and/or the tool inside the lumen). Alternatively or additionally a carriage may be self propelled (for example by an actuator). For example, the carriage and/or the guide and/or the tool may include an actuator. The actuator optionally manipulates the tool and/or the carriage. The actuator may be controlled, for example by an on-board processor and/or a remote control unit.

In some embodiments, a carriage and/or tool may be introduced into the working channel and/or connected to the guide from the proximal end of the catheter. Alternatively or additionally, a carriage and/or tool may be introduced into the working channel and/or connected to the guide from the distal end of the catheter.

In some embodiments, a guide may include a track and/or a guide channel. A guide optionally communicates with the working channel and/or the outer surface of the catheter. Optionally a guide and/or guide channel may communicate with the outside of the catheter at its distal and/or proximal end. Optionally, a guide and/or guide channel may communicate with the outside of the catheter along its length. For example the guide may include a narrow guide channel with a longitudinal slit communicating with the working channel and/or the outer surface of the catheter. Optionally the slit connects to a distal opening and/or to a proximal opening of the guide and/or guide channel and/or working channel. Alternatively or additionally, the slit may run along central and/or interior portion of the guide channel and/or guide. Optionally a slit runs the entire length of the guide and/or guide channel (for example from a distal end and/or a distal opening to a proximal end and/or a proximal opening). For example, a guide may communicate with the working channel and/or the outside of the catheter along its entire length and/or along a length less than the width of the working channel and/or along a length ranging between the width of the working channel to ten times the width of the working channel and/or along a length ranging between ten times the width of the working channel to one hundred times the width of the working channel and/or over a length less than one tenth the length of the catheter and/or over a length between a tenth to a quarter of the length of the catheter and/or over a length ranging between a quarter to a half the length of the catheter and/or between half the length to the entire length of the catheter. For example, a working channel may communicate with one or more guides and/or the outside of the catheter along its entire length and/or along a length less than the width of the working channel and/or along a length ranging between the width of the working channel to ten times the width of the working channel and/or along a length ranging between ten times the width of the working channel to one hundred times the width of the working channel and/or over a length less than one tenth the length of the catheter and/or over a length between a tenth to a quarter of the length of the catheter and/or over a length ranging between a quarter to a half the length of the catheter and/or over a length ranging between a half to the entire the length of the catheter. The width of a communication slit may range for example between\ 0.01 to 0.1 mm and/or between\ 0.1 mm to 0.5 mm and/or between\ 0.5 mm to 2 mm and/or 2 mm or more. The width of a communication slit may range for example between 1/50 to 1/10 the width of the smaller channel and/or between 1/10 to ⅕ the width of the smaller channel and/or between ⅕ to ½ the width of the smaller channel and/or from ½ to the entire width of the smaller channel. In some embodiments the width of the slit may be variable along the length of the channel.

In some embodiments, the guide may include a track running along the working channel and/or an outer surface of the catheter. The guide may optionally extend along the entire length of the working channel and/or a portion thereof. The guide may communicate with the working channel along the entire length of the working channel and/or a portion thereof. In some cases a plurality of guides may all have similar width and/or form. Alternatively or additionally, each guide may have a different width and/or form. In some embodiments, a guide may have a cross sectional area ranging between ½ to ⅕ the cross sectional area of an associated working channel and/or between ⅕ to 1/20 the cross sectional area of an associated working channel and/or between 1/20 to 1/400 the cross sectional area of an associated working channel.

In some embodiments, a narrow guide channel may be used as a guide and/or used as an alternative working channel. For example, a narrow tool may be introduced into or out of the lumen through a guide channel and/or materials may be passed into or out of the lumen through a guide channel.

In some embodiments a catheter may include and/or not include one or more working channels, internal guides (for example communicating with one or more working channels), external guides (for example communicating with an external surface of the catheter), and/or closed guides. Optionally, a working channel may include a longitudinal opening and/or may be closed. A guide channel may interconnect two channels (for example working channels and/or guide channels) and/or may connect a channel to an external opening.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways. Similar steps and/or components and/or aspects may be labeled with the same numbering in multiple Figs. Descriptions of options, components, steps and/or aspects with regard to one Fig. apply also to similarly labeled options, components, steps and/or aspects of other Figs.

In some embodiments, a tool deployed on the outside of a catheter may be supplied with shielding. For example a shield may protect internal structures in the patient from damage and/or puncturing by the tool. For example a balloon may be mounted on the tool. Optionally, the tool may be conveyed through the catheter with the balloon in a contracted (for example deflated) state and/or the balloon may be expanded (for example) inflated while the tool is located outside the catheter.

In some embodiments, a working channel is used for transporting tools and/or materials to and/or from the lumen. For example the working channel may include the access path. Alternatively or additionally, the working channel may be included in a catheter. For example, the catheter may be positioned along an access path to the lumen. A guide may be used, for example, to convey one or more tools to the lumen and/or to control the one or more tools in the lumen (for example controlling a tool may include manipulating and/or supporting and/or directing the tool). For example, the tool may be deployed and/or retrieved and/or conveyed through the working channel via the guide. Alternatively or additionally, the tool may be controlled in the lumen via the guide. For example, a tool may be deployed in a lumen connected to a distal end of a guide. Alternatively or additionally, a tool may be directed along a guide to a location in the lumen where the tool can be supported (permanently or temporarily) by a connection to a body part. For example, the tool may remain connected to the guide and/or the tool may be disconnected from the guide. In some embodiments a tool may be deployed inside the lumen such that an opening of the working channel remains clear. Optionally, clearing the opening of the working channel may facilitate use of the working channel to pass further tools and/or materials into and/or out of the lumen. Optionally, the tool may be reconnected to the guide and/or the tool may be returned to the working channel and/or retrieved from the lumen through the working channel. Optionally, returning and/or retrieving the tool may include directing the tool via a guide to the working channel and/or conveying the tool along the working channel via the guide. Optionally, the tool is returned along the same guide that was used to deploy the tool. Alternatively or additionally, the tool may be returned using a different guide. In some embodiments of the current invention, one or more tools may be inserted into a working channel and/or conveyed in a working channel and/or deployed from a working channel and/or retrieved from a working channel without the use of a guide.

In some embodiments the guide may used to convey and/or guide a tool all the way and/or part of the way to a lumen and/or site of treatment. Alternatively or additionally, a tool may arrive to the site of treatment without the guide and/or the guide may be used to retrieve the tool. Alternatively or additionally, a tool may arrive to the site of treatment without the guide and/or the guide may be used to control the tool at the site of treatment.

In some embodiments, a working channel may include a narrowing at one or more locations. In some embodiments, the lumen may include a portion of the urinary tract, the digestive tract and/or the pulmonary tract.

In some embodiments a plurality of guides may be used to control a plurality of tools. For example a plurality of guides may be positioned along the access channel and/or a respective tool connected to each guide. Optionally, each tool is conveyed and/or directed and/or manipulated and/or supported independently.

In some embodiments, a single guide may be used for multiple tools. For example, a first tool may be disconnected from the guide and/of subsequently a second tool may be connected to the guide. For example, the first tool may be deployed into the lumen and/or disconnected from the guide. Subsequently, the second tool may be connected to the guide. Alternatively or additionally, the first tool may be returned from the lumen and/or retrieved from the working channel and/or then disconnected from the guide. Subsequently the guide is optionally used for a second tool. Alternatively or additionally, multiple tools may be connected to a single guide and/or a single tool may be connected to multiple guides.

In some embodiments a tool may block a channel and/or a path. Optionally, the tool may be moved to unblock the path and/or channel. For the sake of the current disclosure, unless stated otherwise blocking a path and/or channel is relative to a context. For example, in the context of passing a sequence of tools through a channel, a first tool blocks a channel when the second tool is prevented from passing through the channel due to the first tool. Unblocking the channel may mean allowing the second tool to pass through the channel. Alternatively or additionally, blocking may mean preventing a object from passing along the path when the cross sectional area ranges for example between 10% to 25% of the cross sectional area of the channel and/or path and/or 25% to 50% of the cross sectional area of the channel and/or path and/or 50% to 75% of the cross sectional area of the channel and/or path and/or 75% to 100% of the cross sectional area of the channel and/or path. Optionally, for the sake of this disclosure unblocking a path and/or channel may mean opening 10 to 25% of the blocked cross section of the path and/or channel and/or opening 25 to 50% of the blocked cross section and/or opening 75 to 100% of the blocked cross section.

In some embodiments, a tool may be inserted into a catheter and/or retrieved from the catheter and/or reinserted into the catheter while the distal end of the catheter remains in a lumen of a patient. For example inserting, retrieving and/or reinserting may be used to reposition a tool and/or a camera in a lumen. For example a camera may be relocated to a new position for example with an improved view and/or a tool may be placed in a new position for example with better access to a treatment location.

### EXEMPLARY EMBODIMENTS

### Method Controlling Tool Inside a Lumen

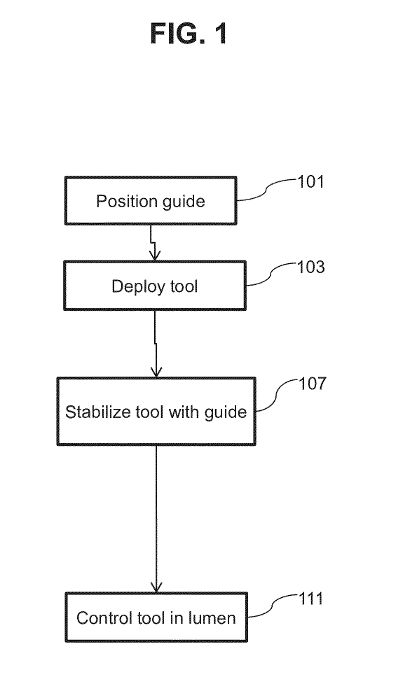

Referring now to the drawings, FIG. 1 illustrates a method of controlling a tool inside a lumen accessible via a working channel in accordance with some embodiments of the present invention. In some embodiments, a guide may be positioned 101 along a working channel. For example, one or more guides and/or working channels may be included in a catheter. Optionally, a tool is deployed 103 into the lumen through a working channel. Optionally, the guide is used to stabilize 107 and/or to control 111 the tool in the working channel and/or in the lumen (for example to stabilize the tool and/or support the tool and/or manipulate the tool).

In some embodiments, a catheter including one or more guides and/or a working channel may be positioned 101 along the path to a treatment site. For example a proximal opening of the catheter may be accessible the outside of the body and/or a distal opening may be exposed to the lumen. One or more tools may be conveyed along a working channel of the catheter and/or deployed 103 out the distal opening into the lumen. The guides may be used for example to convey the tools along the working channel.

In some embodiments, a guide may be used to stabilize, manipulate and/or control a tool in the lumen. For example, after a tool is deployed 103 into the lumen, the tool may remain connected to a guide. Alternatively or additionally, the guide may be used to deploy 103 the tool to a target site in the lumen. Optionally, the tool may be disconnected from the guide temporarily and/or permanently while the tool is in the lumen.

### Method of Sharing a Working Channel to a Lumen

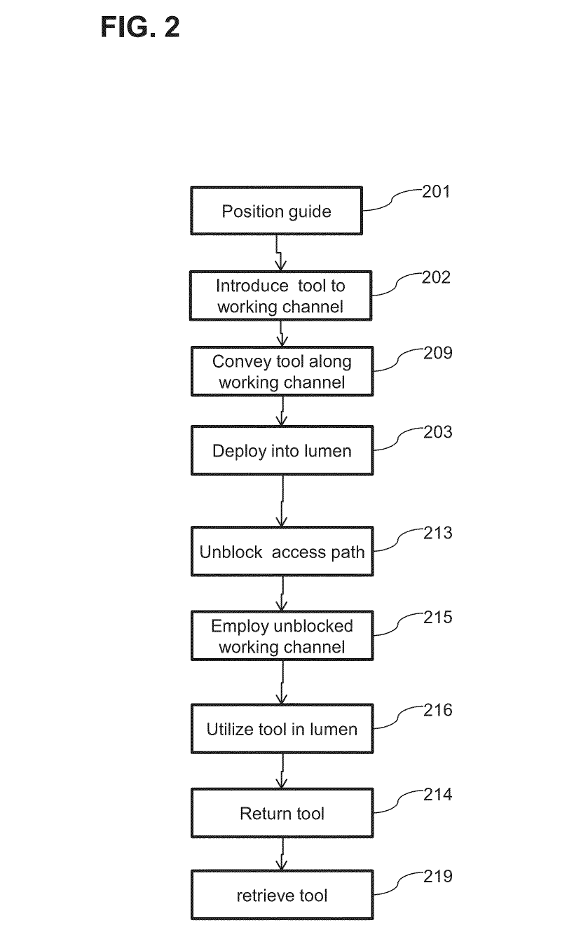

FIG. 2 illustrates a method of sharing a working channel in accordance with some embodiments of the present invention. Optionally, one or more guides may be positioned 201 along the working channel. A guide will optionally be narrow in comparison to the working channel. For example, while positioned 201, a guide may leave enough of the working channel free to allow the working channel to be employed 215 for various functions such as passing objects and/or fluids into and/or out of the lumen. In some embodiments, the guide and/or the working channel may be part of a catheter.

In some embodiments, the guide may be used by a user at a proximal end of the working channel to control a tool at a distal end of the working channel while the working channel remains clear for use for another function. Alternatively or additionally, the guide may be used and/or perform one function while the working channel remains clear for use for another function. For example, tools and/or fluid may be transported directly through the working channel to the lumen.

In some embodiments, a guide may be used to convey 209 a tool along a working channel and/or unblock 213 the working channel. For example, a first tool may be introduced 202 into the working channel. In some embodiments, the first tool may be large and/or may block the working channel. Optionally, a guide may be used to convey 209 the first tool along the working channel to a widening of the channel and/or a guide may be used to deploy 203 the first tool out from the working channel into the lumen. Conveying 209 the first tool to the widening location and/or deploying 203 the first tool may unblock 213 the working channel. The unblocked 213 working channel is optionally employed 215 for a further function. For example, the unblocked 213 working channel may be employed 215 for transferring a fluid into and/or out of the lumen. Alternatively, the unblocked working channel may be employed 215 for further conveying 209 a different tool to the lumen. Optionally, while the working channel is being employed 215 for a further function, the first tool may be utilized 216 in the lumen.

In some embodiments, while the tool is in the lumen it may remain attached to the guide. For example the guide may stabilize the tool and/or the guide may be used to manipulate the tool. Alternatively or additionally, a tool may be disconnected from the guide while it is in the lumen. Alternatively or additionally, the tool may be conveyed along the working channel without the guide. Alternatively or additionally, the tool may be connected to a guide when it is deployed into the lumen.

In some embodiments, a tool may be returned 214 from the lumen. Optionally, the guide will be used to return 214 a tool from the lumen. For example a tool may remain connected to the guide in the lumen. The tool may be returned 214 from the lumen using the guide. Alternatively or additionally, a tool may be connected and/or reconnected to a guide while in the lumen. Alternatively or additionally, the tool may be returned 214 from the lumen without the guide (for example the tool may be connected to a tether and/or drawn into the working channel by suction). Optionally, the tool be connected and/or be reconnected to the guide in the working channel. The guide is optionally used to retrieve 219 the tool, for example by conveying the tool along the working channel to outside the body.

States of Tool and/or a Working Channel

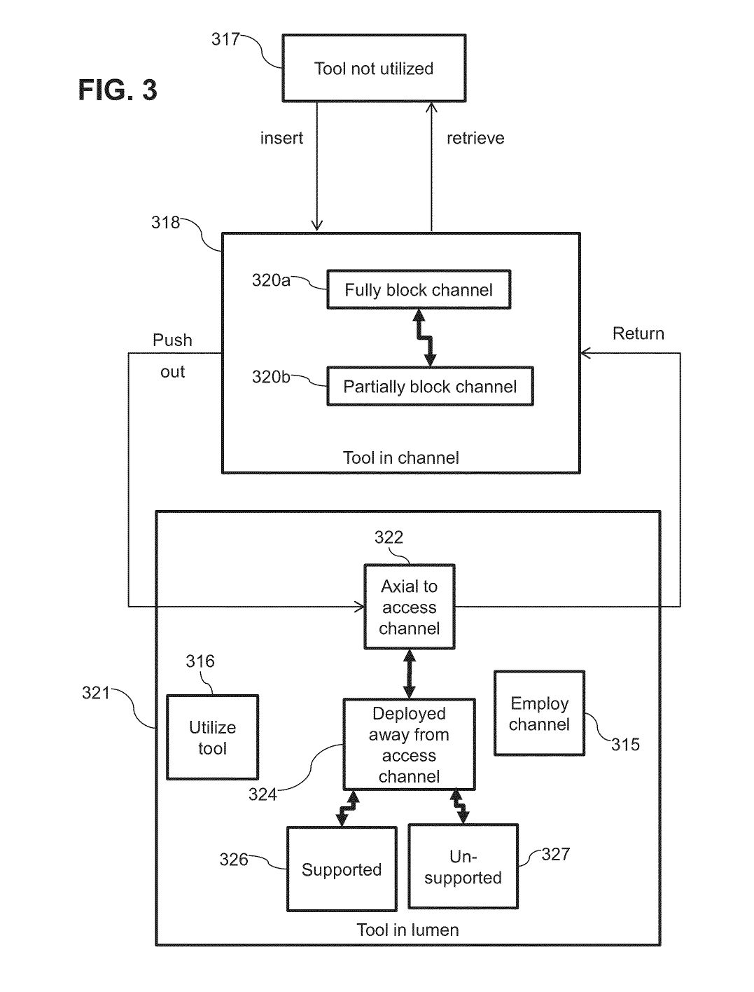

FIG. 3 is a state chart illustration of states of a tool in accordance with some embodiments with the current invention. Optionally, the tool may be connected to a guide in one, some, all and/or any of its states and/or a guide may be used to transport a tool between states.

In some embodiments, a tool may be located outside a patient while not being utilized 317. The tool may be inserted into a working channel 318. The working channel may include an access path to the lumen. For example, while located in the working channel, the tool may fully block 320a and/or partially block 320b the working channel.

In some embodiments the tool may be pushed out of the working channel into the lumen 321. In the lumen the tool may remain for example axial to the working channel 322 possibly blocking access to the channel. Optionally, the tool is deployed 324 away from the entrance of the channel possibly unblocking the channel. Optionally, tool is supported 326 by external means in the channel (for example by attachment to an organ of the patient and/or to by attachment to an external wall of a catheter). Alternatively or additionally, the tool may be unsupported 327 in the lumen (for example the tool may float freely in the lumen and/or the tool may be supported by the guide).

In some embodiments, the tool may be utilized 316 in the channel. Optionally, the working channel may be employed 315 for further functions, such as transporting materials and/or conveying further tools into the lumen. For example, a first tool may be conveyed through the channel to the lumen. Optionally, when first tool is deployed in the lumen, unblocking the channel, the channel may be employed to transport fluid and/or convey another tool into and/or out of the lumen.

In some embodiments a tool may be returned to the working channel and/or retrieved back out of the patient.

### System to Control a Tool Inside a Lumen Having a Narrow Access Path

In some embodiments of the current invention, a guide may be placed along an access path to a site inside a patient. For example, the guide 432a may be used to direct and/or convey a carriage 430a. Optionally the carriage 430a may be operationally connected to a tool 434. In some embodiments, the carriage 430a may stabilize the tool 434 in a lumen 456 at a site of treatment or examination. For example, where the lumen 456 is large and/or there is a high flow and/or a lot of movement in the lumen, it may be useful that the tool 434 remain connected to the guide 432a for support. Alternatively or additionally, carriage 430a may convey tool 434 to and/or from the site. For example when the path to the site is complex and/or narrow it may be advantages to use guide 432a to convey tool 434 to lumen 456. In some embodiments, the carriage 430a may be used to manipulate tool 434 according to commands from outside the patient.

In some embodiments an endoprobe 442a (for example an endoscope and/or a catheter) may be used to place guide 432a along all and/or part of a path from outside the patient to a treatment and/or examination site. Carriage 430a optionally moves along guide 432a. For example guide 432a may include a channel and/or a track. Optionally, carriage 430a is controlled from outside the patient. For example, a manipulator rod may run along the guide 432a from carriage 430a to a person who controls carriage 430a and/or tool 434 from outside the patient.

In some embodiments tool 434 may be connected to carriage 430a and/or carriage 430a may be connected to guide 432a before the tool is inserted into the patient. Alternatively or additionally, tool 434 may be connected to carriage 430a and/or carriage 430a to the guide 432a while tool 434 and/or carriage 430a is on the path to the site. Alternatively or additionally, the tool 434 may be connected to carriage 430a and/or carriage 430a to guide 432a while tool 434 and/or carriage 430a is in lumen 456. For example tool 434 may connected to guide 432a while in lumen 456 and/or guide 432a may be used to retrieve tool 434 from lumen 456.

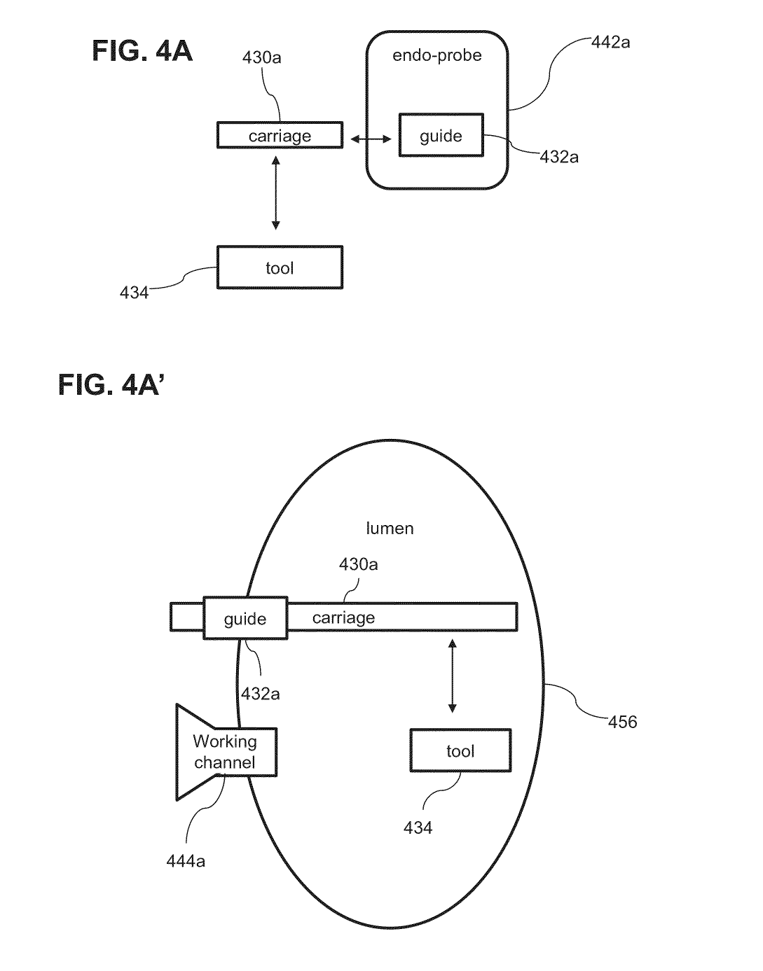

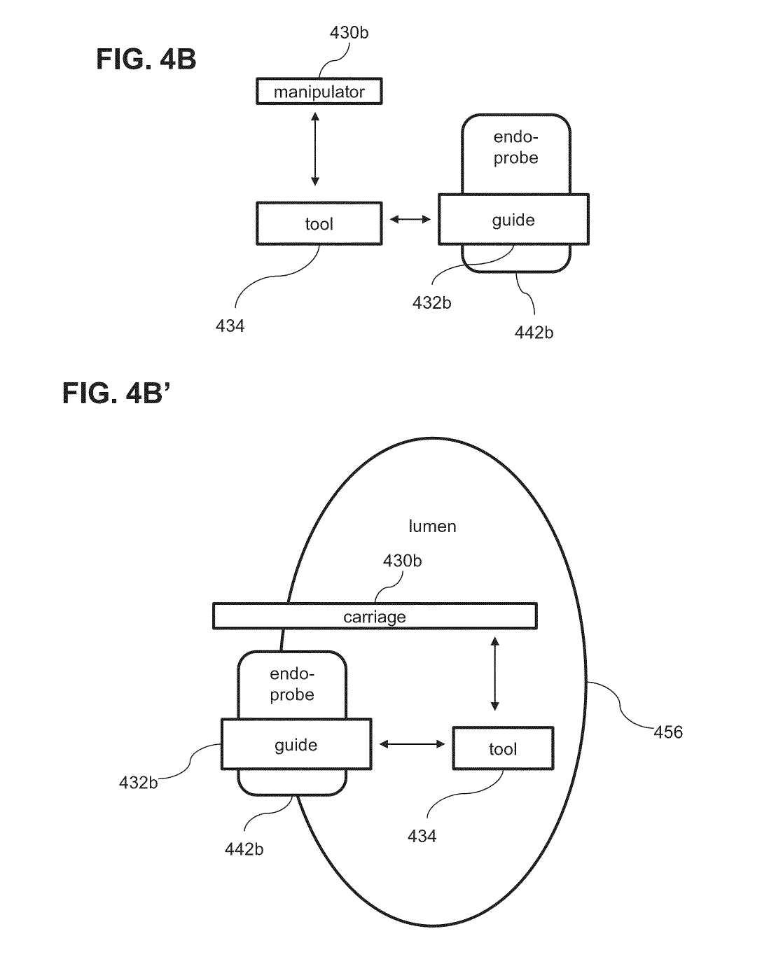

FIG. 4A′ is a block diagram illustrating tool 434 in lumen 456 with guide 432a in accordance with some embodiments of the current invention. Optionally, after guide 432a is placed in lumen 456 and/or the path thereto, endoprobe 442a that was used to place guide 432a is removed. For example, guide 432a may be used to direct and/or convey carriage 430a. In some embodiments, tool 434 may be inserted into lumen 456 through a working channel 444a and/or connected to carriage 430a inside lumen 456. For example working channel 444a may be temporary and/or may take a path different from guide 432a. For example, the tool 434 may be inserted through an incision which may be closed after tool 434 has been inserted. Optionally, the guide 432a may be used to return tool from lumen 456 without retrieving the tool 434 from the patient. For example, tool 434 may be returned to a position inside the patient where tool 434 can be left without doing harm and/or where tool 434 will eventually be expelled from the patient.

FIGS. 4B and 4B′ are block illustrations of a system for guiding tool 434 inside a patient in accordance with some embodiments of the invention. Optionally, tool 434 may be connected to a guide 432b directly and/or via a carriage. A manipulator 430b may be used to manipulate tool 434 while it is inside the patient. For example, manipulator 430b may be physically connected to tool 434 (for example manipulator 430b may include a wire). Alternatively or additionally, a manipulator 430b may be located outside the body of the patient (for example a magnet and/or a radio control unit). Guide 432b may be used to steady and/or direct and/or convey tool 434. In some embodiments an endoprobe 442b may be used to place guide 430b. Optionally, guide 430b may be a permanent part of endoprobe 442b. Alternatively or additionally, guide 430b and endoprobe 442b may be separate and/or independent.

### Camera for Use Inside a Lumen

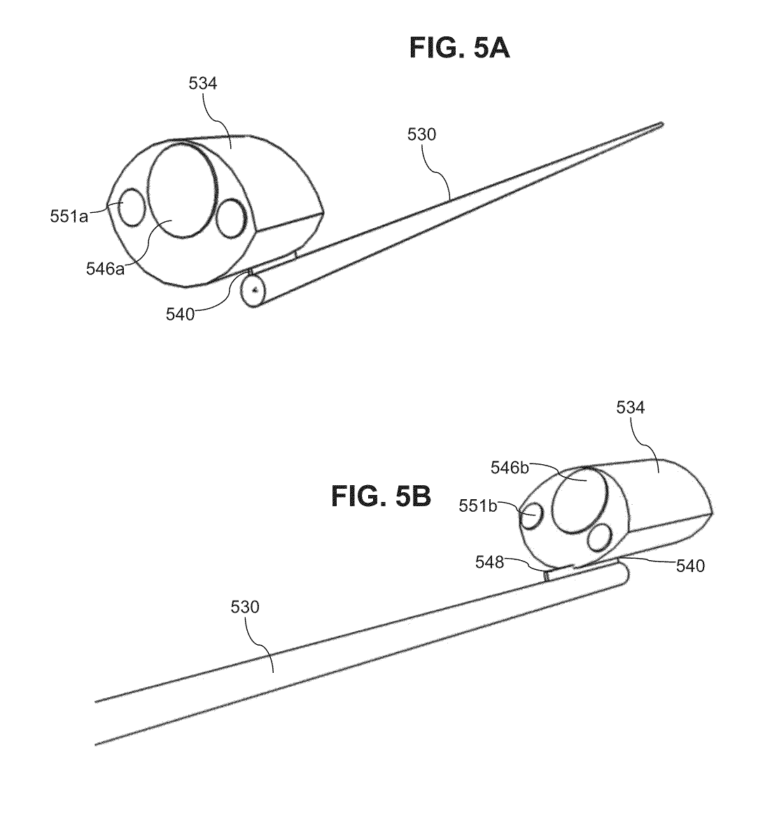

FIGS. 5A and 5B illustrate two perspective views of a tool 534 for use in a lumen of a patient in accordance with some embodiments of the present invention. For example tool 534 may include one or more cameras 546a,b and/or one or more light sources 551a,b. Tool 534 is optionally mounted on a carriage 530. Optionally carriage 530 may connect to a guide. For example, a guide may direct and/or convey and/or stabilize carriage 530 and/or tool 534 inside a working channel and/or inside a lumen, for example as explained herein below. For example, tool 534 is connected to carriage 530 off center of tool 534 (tool 534 and carriage 530 are not joined concentrically).

In some embodiments, carriage 530 may include a flexible and/or elastic rod and/or wire. For example, carriage 530 may be used to push and/or pull tool 534 along an access pathway (for example a working channel) to a lumen. Carriage 530 is optionally flexible enough to follow curves in the working channel, but stiff enough to retain its length (for example without folding over and/or significantly stretching). In some embodiments a carriage may be torsion resistant. For example, a user may twist a proximal end of the carriage in order to rotate the camera at the distal end.

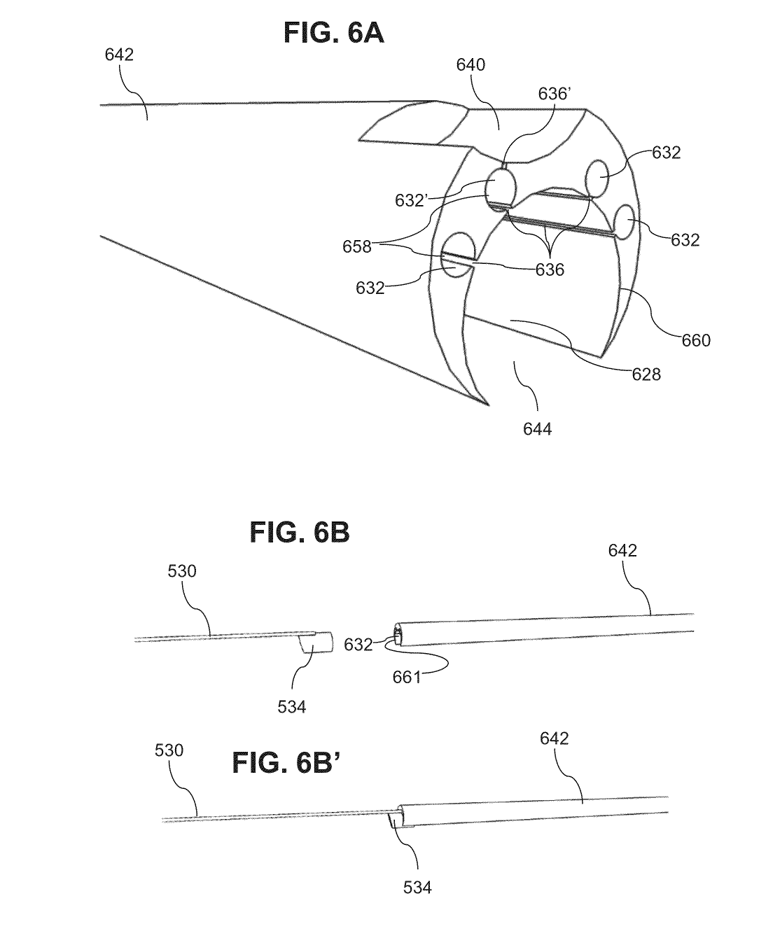

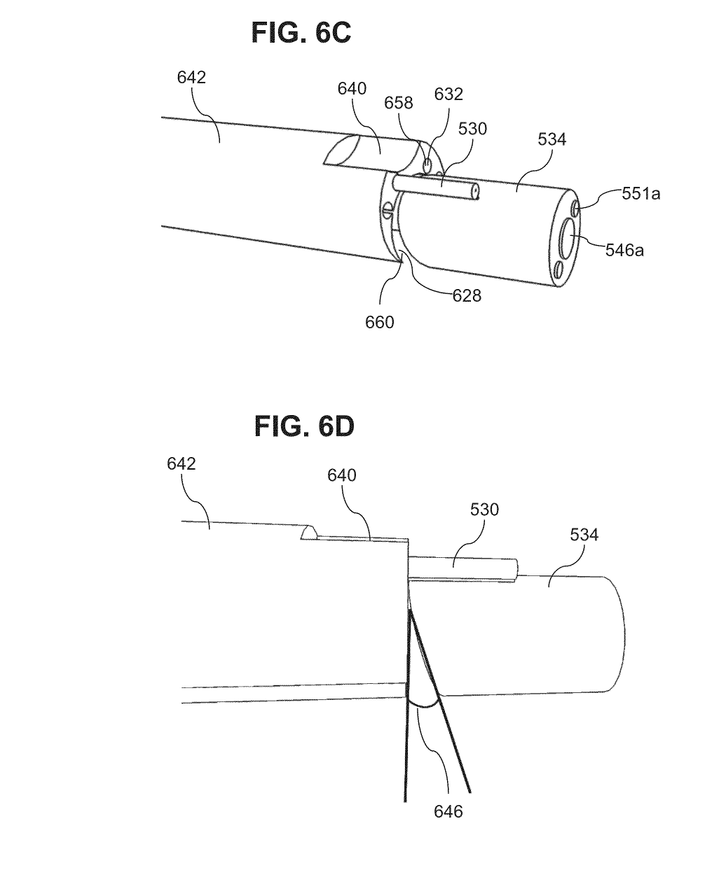

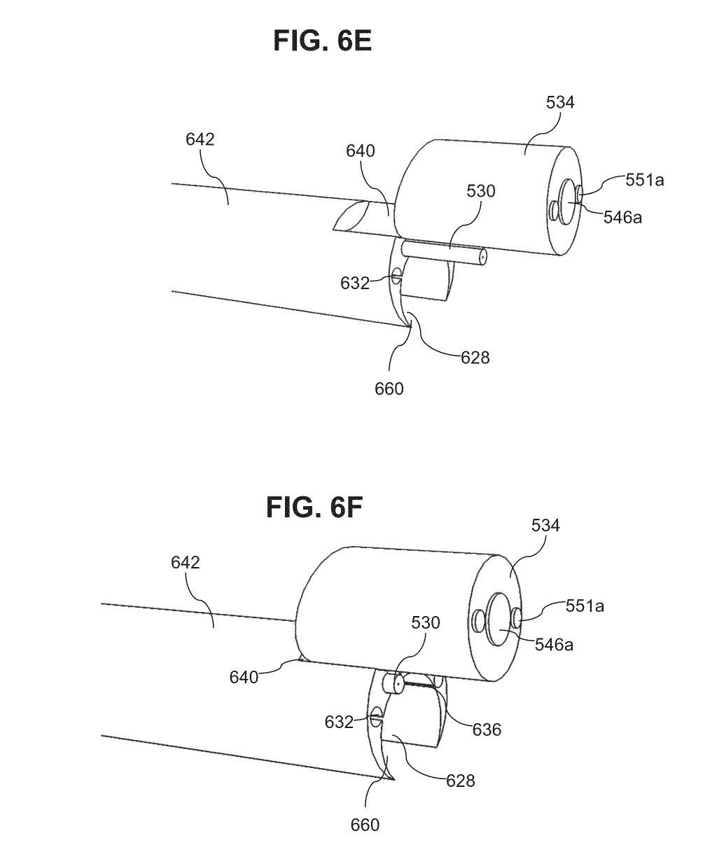

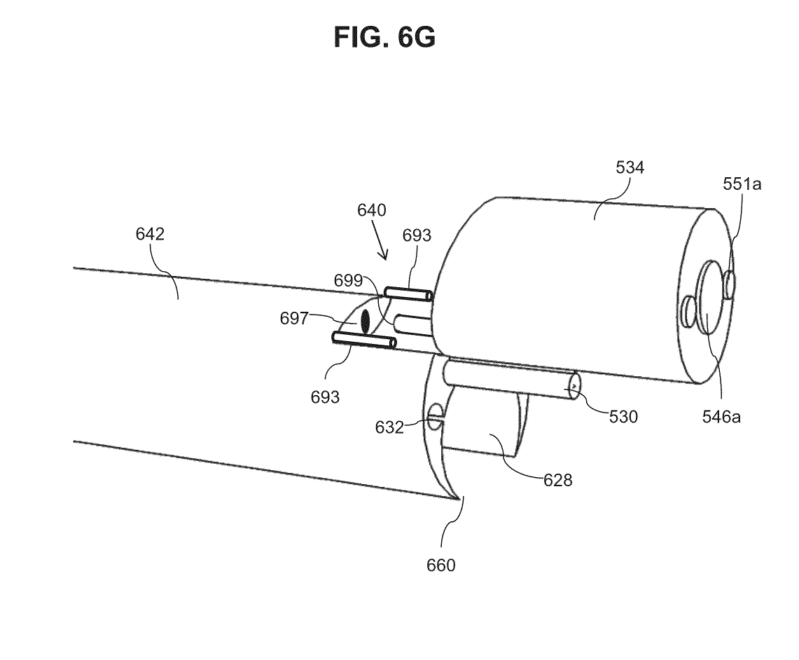

In some embodiments, tool 534 may be joined to carriage 530 by a mount 540. Carriage 530 and/or tool 534 may include an orientor 548. For example, orientor 548 may orient and/or stabilize tool 534 with respect to a guide and/or with respect to a catheter and/or with respect to another tool (for example as illustrated in FIGS. 6A-F). In the embodiment of FIGS. 5A, B, for example, orientor 548 and mount 540 are collinear and directed radially from the edge of carriage 530. Optionally a stabilizer and/or orientor may have different orientations and may be directed non-radially with respect to a carriage.

In some embodiments, tool 534 is permanently connected to mount 540 and/or carriage 530. Alternatively or additionally, a mount may be reversibly connected to the tool. For example the tool may be connected and/or disconnected and/or reconnected to the carriage and/or guide and/or manipulator. Optionally, tool 534 is directly and/or physically attached to carriage 530 and/or mount 540. Optionally or additionally, a tool may be connected to a carriage by a magnet or other indirect method.

Inserting a Tool into a Catheter with Guides

FIGS. 6A-F illustrate insertion and deployment of a tool with a catheter in accordance with an embodiment of the current invention. Optionally, a catheter includes an outer sleeve 642. Outer sleeve 642 optionally includes one or more large working channels 628. In some embodiments sleeve 642 includes one or more elongated guides 632 and/or 632′. For example, guides 632 may include narrow guide channels. Optionally guides 632, 632′ may be oriented parallel to working channel 628 (for example they may run along the length of working channel 628). Optionally guides 632 and 632′ may communicate with working channel 628 and/or with a region on the outside of the catheter. For example guides 632 and/or 632′ may convey carriage 530 and/or tool 534 along working channel 628 and/or outside the catheter. Optionally, outer sleeve 642 includes a mount 640. For example, mount 640 may be used to steady and/or support a tool and/or hold a tool rigidly to a catheter, for example tool 534. Optionally a mount may include a guide channel and/or a track and/or a hole and/or a pin and/or a peg.

In some embodiments, one or more of guides 632 and 632′ may be distributed around working channel 628. Optionally, a guide 632 runs the entire length of working channel 628. Alternatively or additionally, guide 632 may run along a portion of the length of the working channel 628. Optionally, a guide 632 communicates with working channel 628 and/or the outside of the catheter along its entire length and/or along a portion of its length.

In some embodiments, a guide 632 may include a guide channel. For example a guide channel may be narrower than the working channel 628. Communication between guide 632 and working channel 628 is optionally supplied by a longitudinally communication opening 636 for example in the form of a slit between guide 632 and working channel 628.

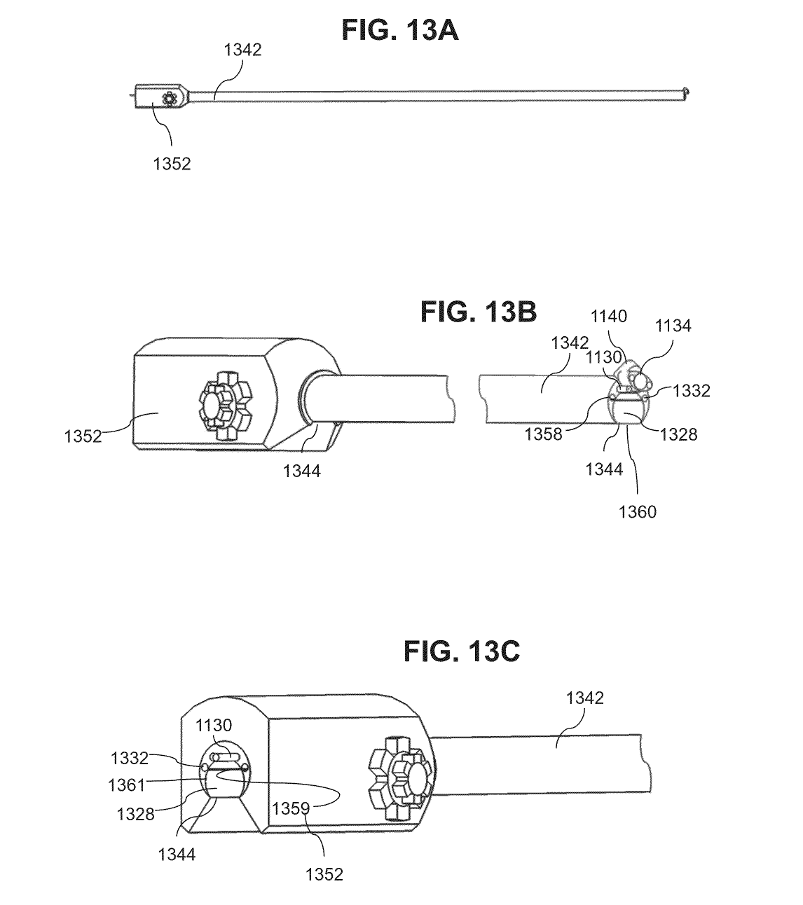

In some embodiments, a guide 632 and/or a working channel 628 may communicate with a space outside the catheter. Optionally a guide or guide channel and/or a working channel may communication with the outside of the catheter at one or both of the distal and/or proximal ends. For example a guide channel (for example guide 632) may include a distal opening 658 and/or a proximal opening (for example see proximal opening 1359 of FIG. 13C). For example working channel 628 may include a distal opening 660 and/or a proximal opening (for example see proximal opening 661 of FIG. 6B). Alternatively or additionally, one or both ends of a guide channel and/or working channel may be closed.

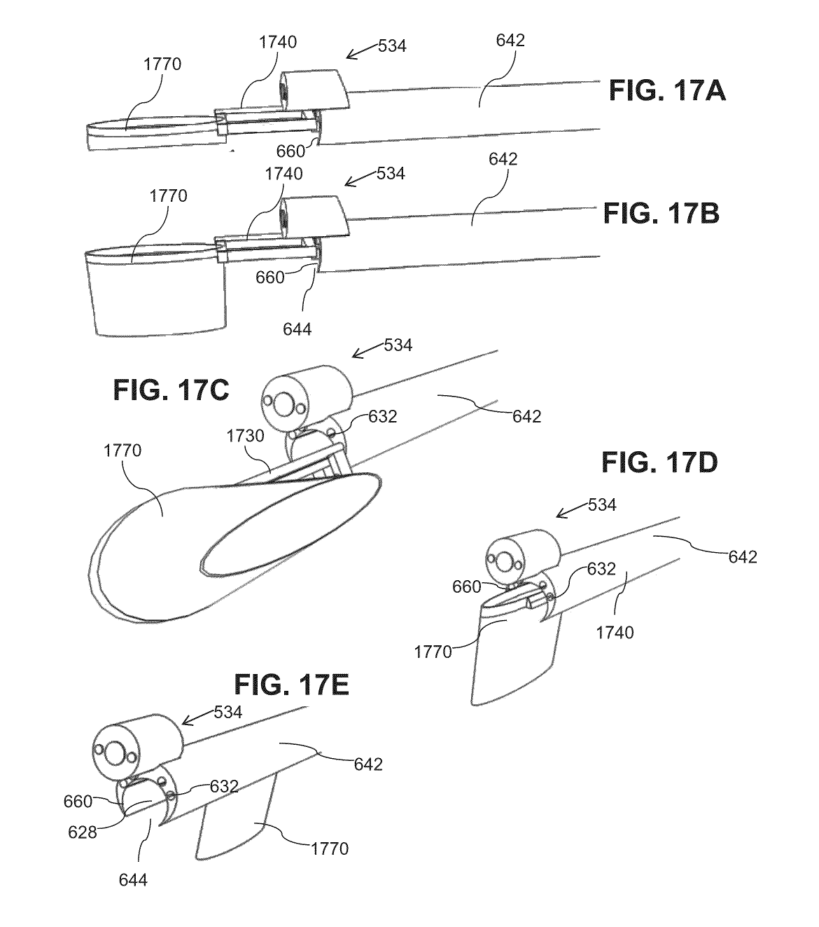

In some embodiments, a guide and/or a working channel may communicate with the space outside the catheter along all or part of its length. For example guide 632′ communicates with the outside of the catheter along a portion of its length via a communication opening 636′. For example guides 632 and 632′ communicate with the working channel 628 along their entire length via openings 636. Optionally openings 636 connect to distal openings 658 at the distal ends of respective guides 632 and 632′. Optionally, working channel 628 communicates with the exterior of the catheter along its length via a slit, for example longitudinal opening 644. For example longitudinal opening 644 may allow movement of large objects using working channel (for example as illustrated in FIGS. 17A-17D). For example longitudinal opening 644 may facilitate insertion of outer sleeve 642 around an endoprobe and/or longitudinal opening 644 may facilitate conveyance of a large object. Optionally the longitudinal opening 644 is biased open.

FIGS. 6B and 6B′ illustrate insertion of a tool into a catheter in accordance with an embodiment of the current invention. For example tool 534 is inserted into proximal opening 661 of outer sleeve 642. Optionally, carriage 530 is inserted into guide 632′. Optionally mount 540 passes through opening 636′ connecting tool 534 to carriage 530. Alternatively or additionally, a guide may be closed (without a slit) and/or a carriage passing through the guide may be connected to the tool via a magnet and/or another means. Note that longitudinal opening within a single channel may be oriented at right angles (90°) and or opposite (180°) and/or at an obtuse angle (>90°) and/or at an acute angle (<90°).

In some embodiments a guide and/or carriage may be used to manipulate and/or convey a tool inside a working channel. For example, carriage 530 may be used to push and/or convey tool 534 through working channel 628. For example, in FIGS. 6C and 6D, carriage 530 has been pushed from proximal opening through guide 632′ until carriage 530 reaches and/or extends out of distal opening 658. Optionally carriage 530 pushes tool 534 through working channel 628 from proximal opening 661 until it reaches and/or extends out of distal opening 660.

In some embodiments, a carriage and/or guide may be used to deploy and/or steady and/or support a tool outside of a catheter. For example in FIG. 6C tool 534 and/or a distal end of carriage 530 and/or mount 540 and/or orientor 548 have been extended and/or deployed out distal openings 658 and 660 of guide 632′ and/or opening 636 and/or channel 628 respectively. Optionally, a clearance 646 is provided between tool 534 and the distal end of the catheter. Optionally a center portion of carriage 530 remains inside guide 632′ and/or a proximal portion of carriage 530 extends out proximal opening of guide 632′.

In some embodiments, a carriage and/or guide may be used to manipulate a tool outside of a catheter. For example, after extending carriage 530 and/or tool 534 out of the distal end of the catheter, one/or both may be rotated into the configuration of FIG. 6E and/or retracted to the configuration of FIG. 6F. A user (for example a doctor performing a medical procedure with the catheter), may optionally twist the proximal end of carriage 530. For example, twisting carriage 530 while it is supported inside guide 632′ and/or while the distal end of carriage 530 and/or tool 534 are extended out of working channel 628 may move tool 534 away from the axis of working channel 628 and/or clearing distal opening 660 (moving out of at least 80% of the cross section of distal opening 660). Optionally, working channel 628 is unblocked and/or can be used for a further function. In the deployed position of FIG. 6F, the lens 546b of tool 534 is beyond the outer cross section of sleeve 642. For example, this allows a camera in tool 534 to see backward without being blocked by sleeve 642.

In some embodiments, a tool may be supported and/or steadied on an outside surface of a catheter. For example, in FIG. 6F, carriage 530 is used to pull tool 534 onto mount 640. Optionally, the distal end of carriage 530 is retracted at least partially back into guide 632′ (for example by a user pulling on the proximal end of carriage 530). Optionally, tool 534 is pulled back onto mount 640. Optionally, mount 540 and/or orientor 548 are pulled into slit communication opening 636′ orienting, supporting and/or steadying tool 534 on the outer surface of outer sleeve 642. Optionally, carriage 530 remains inside of guide 632′ retaining tool 534 in place on mount 540. Optionally, subsequently carriage 530 may be used to push tool 534 back off of mount 540 and/or to return tool 534 back into working channel 628 and/or to retrieve tool 534 out proximal opening 661 of working channel 628. Optionally, while tool 534 is steadied on mount 640 carriage 530 may be disconnected and/or retracted out of guide 632′. Optionally, while tool 534 is steadied on mount 640 carriage 530 may be reconnected to tool 534 for example to return tool 534 to working channel 628.

In some embodiments, the width of mount 540 may be the same as, slightly bigger than and/or slightly smaller than the width of communication opening 636. For example opening 636 may be slightly wider than mount 540 and/or opening 636′ may be slight narrower than mount 540. Optionally mount 540 fits loosely into opening 636 and/or can be easily conveyed along channel 628. Optionally mount 540 fits tightly into opening 636′ and/or tool 534 is held securely on mount 640.

In some embodiments, the height of mount 540 may be the same as, slightly bigger than and/or slightly smaller than the wall thickness of guide 632′ at communication opening 636. For example the wall thickness of guide 632′ at communication opening 636 may be slightly smaller than the height of mount 540 and/or the wall thickness of guide 632′ may be slight larger than the height of mount 540. Optionally tool 534 has clearance from opening 636 and/or can be easily conveyed along channel 628. Optionally tool 534 fits tightly onto opening 636′ and/or tool 534 is held securely on mount 640. For example, the thickness of mount 540 may range for example from 0.01 to 0.1 mm and/or from 0.1 mm to 0.5 mm and/or from 0.5 mm to 2 mm and/or 2 mm or more. For example the height of mount 540 may range for example between 0.1 mm to 0.5 mm and/or between 0.5 mm to 2 mm and/or between 2 mm or more. For example wall thickness of guide 632′ may range for example between 0.1 mm to 0.5 mm and/or between 0.5 mm to 2 mm and/or 2 mm or more.

In some embodiments, outer sleeve 642 may be inserted to a location in the subject and then tool 534 may be inserted using working channel 628 as an access channel. Alternatively or additionally, camera tool 534 may be positioned at the distal end of sleeve 642 before inserting sleeve 642 into a subject. Alternatively or additionally, a distal end of sleeve 642 may be inserted partially into the subject (for example past a narrowing of the access path) and then camera tool 534 may be positioned at the distal end of sleeve 642 and/or used to direct the catheter to a location.

FIG. 6G illustrates optional stabilizing features of a tool mount in accordance with an embodiment of the current invention. For example a tool mount may include a hole 697 that fits a peg 699 on the tool. Alternatively or additionally, the hole may be in the tool and the peg in the mount. For example a mount may include a track 693 which fits into a slot in the tool. Alternatively or additionally, the tool may include the track and the mount may include the slot. Optionally mount 640 and/or the stabilizing features may rigidly connect tool 534 to the outside of sleeve 642.

### Sharing a Working Channel

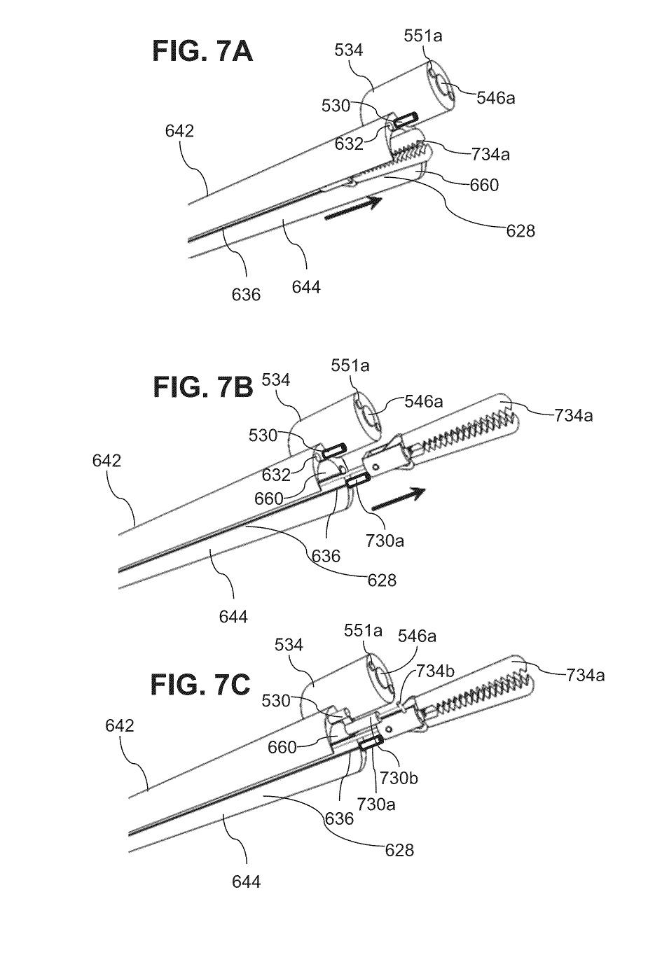

FIGS. 7A-C illustrate sharing of a working channel in accordance with an embodiment of the present invention. For example, multiple guides can be used to independently control multiple tools with a single catheter. Optionally the tools pass serially through a single working channel of a single catheter. For example the multiple tools may be deployed from a working channel and/or manipulated and/or supported simultaneously in a region inside the patient via a single catheter and/or working channel.

In some embodiments, after deploying a first tool, for example tool 534, is deployed in a lumen (for example as described herein above). Subsequently, a second tool, for example tool 734a is optionally conveyed through working channel 628 behind tool 534. Optionally, second tool 734a is connected to a second carriage 730a. Optionally, carriage 730a and/or tool 734a may be connected to a guide 632, separately from tool 534. Optionally, tool 734a is deployed and/or controlled independently from tool 534 (for example using a carriage and/or guide 632).

In some embodiments, a third tool 734b is deployed using another guide 632 and/or carriage 734b.

In some embodiment two tools may function cooperatively. Optionally, the two tools are conveyed to the region and/or controlled independently. For example the two tools may be controlled using different guides. For example, tool 734a includes a forceps that are biased to an open position and tool 734b includes a pushing element to close the forceps of tool 734b. Tools 734a,b may be deployed independently. Optionally tools 734a,b function cooperatively. For example, the forceps may be moved near an object to be grasped and then the pushing element may be used to close the forceps, grasping the object.

### Multi Part Catheter System

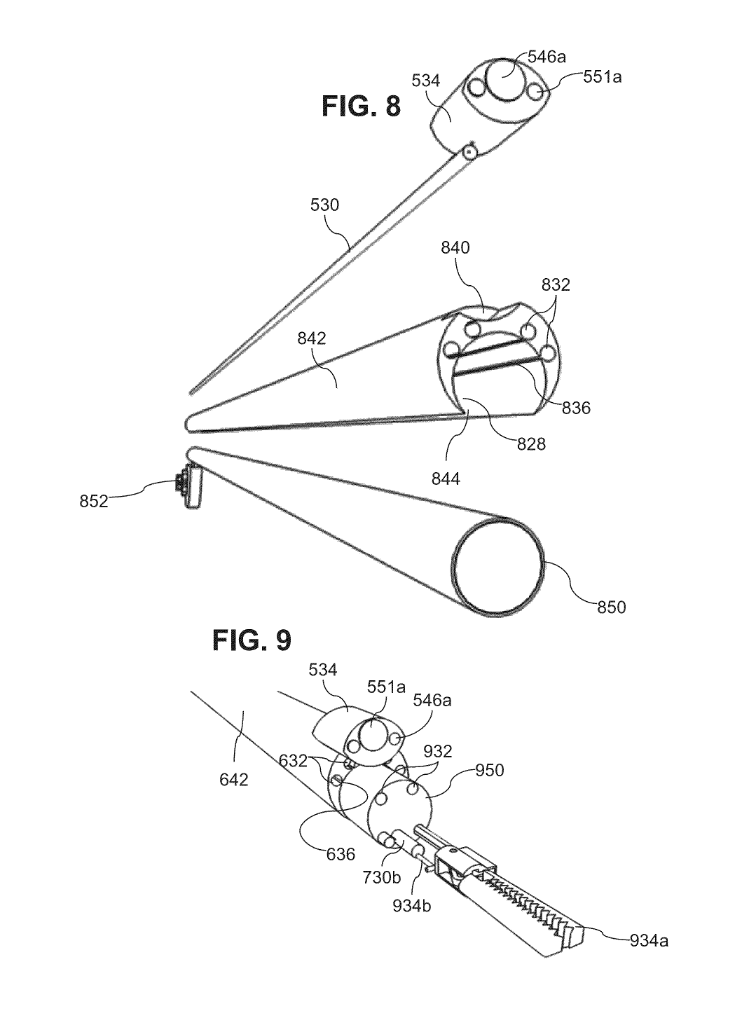

FIG. 8 illustrates a multi part catheter system in accordance with some embodiments of the current invention. For example, a multi-part system may include a passive outer sleeve and an endoprobe. For example outer sleeve may include a flexible tube. An endoprobe 850 may optionally provide a steering mechanism 852. Endoprobe 850, may, for example, fit into a working channel 828 of sleeve 842. Steering mechanism may protrude through a longitudinal opening 844 in a working channel 828. Steering mechanism 852 is optionally used to steer and/or bend a distal end of endoprobe 850 and/or passive sleeve 842.

In some embodiments, endoprobe 850 may be inserted though an access path to a target location in a subject. After endoprobe 850 is in position, sleeve 842 may be slid over endoprobe 850 into the target location (for example with endoprobe fitting into working channel 828 and/or one or more guides). Once sleeve 842 is in position, endoprobe 850 may be retrieved unblocking working channel 828. Once in position with working channel free, sleeve 842 may be used as an access channel for one or more tools for use in the lumen. Alternatively or additionally, the system including sleeve 842 and/or endoprobe 850 and/or a camera (for example tool 534) may be inserted as together to the target site. Alternatively or additionally, part of the transport to the target location may be achieved by endoprobe 850 without sleeve 842, and/or part of the transport may be made by endoprobe 850 and sleeve 842 together. Alternatively or additionally, part of the transport to the target location may be achieved by sleeve 842 and/or endoprobe 850 without a camera, part of the transport may be made by sleeve 842 and/or endoprobe 850 with the camera.

In some embodiments, passive outer sleeve 842 may include one or more guide 832 channels. Communication between guide 832 channels and working channel 828 is optionally provided by longitudinal slits 836. Passive sleeve 842 optionally includes a mount 840 for tool 534.

### Sharing a Working Channel

FIG. 9 illustrates a catheter system in accordance with an embodiment of the present invention. Optionally, a working channel may be shared with a complex tool. For example, an endoprobe 950 may include one or more channels and/or guides 932. For example endoprobe 950 may include multiple simple tools (for example forceps tool 934a and/or a pusher tool 934b). Optionally, working channel 628 of outer sleeve 642 may be used as an access channel for multiple tools, for example for a complex tool for example endoprobe 950 and/or a simple tool for example camera tool 534. Each tool may be inserted and/or deployed and/or retrieved in sequentially and/or simultaneously.

### Communication Between Guide Channels and an Endoprobe

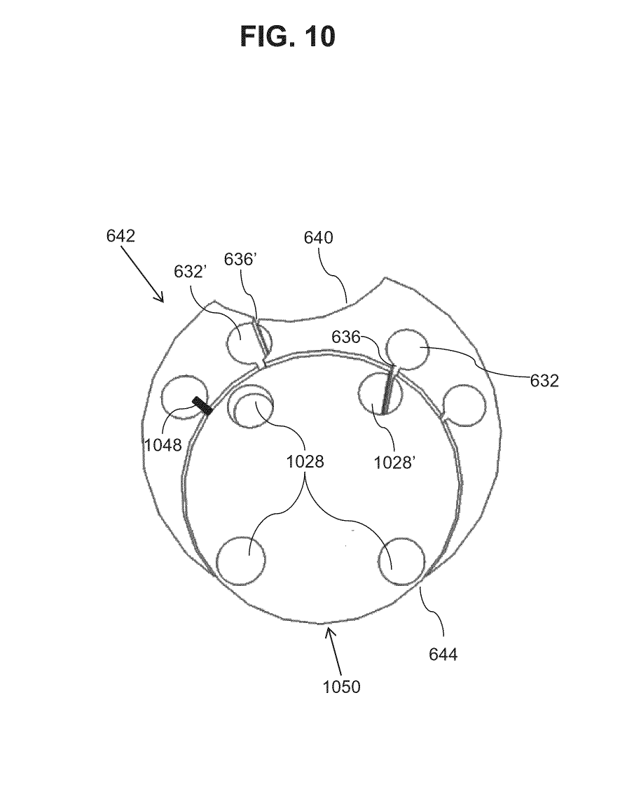

FIG. 10 illustrates a multi channel endoprobe inserted into a working channel of an outer sleeve in accordance with an embodiment of the current invention. Optionally, one or more guides of the outer sleeve may communicate with the endoprobe. For example, the endoprobe may include an orientor and/or a channel that communicates with one or more of the channels of the outer sleeve.

In some embodiments, an orientor may help position an endoprobe and/or a tool inside the working channel of sleeve 642. For example an orientor 1048 may be fixed on an endoprobe 1050. Orientor 1048 may fit into a slit and/or guide of sleeve 642 (for example one of openings 636 and/or guides 632). Orientor 1048 may fix the orientation of endoprobe 1050 with respect to sleeve 642.

In some embodiments, one or more channels of an endoprobe may communicate with a guide channel of an outer sleeve. For example, channel 1028′ of endoprobe 1050 is in communication with guide 632 channel of outer sleeve 642. Optionally, some channels 1028 of endoprobe 1050 are not in communication with the sleeve.

### Stabilizing a Camera on an External Surface of a Catheter

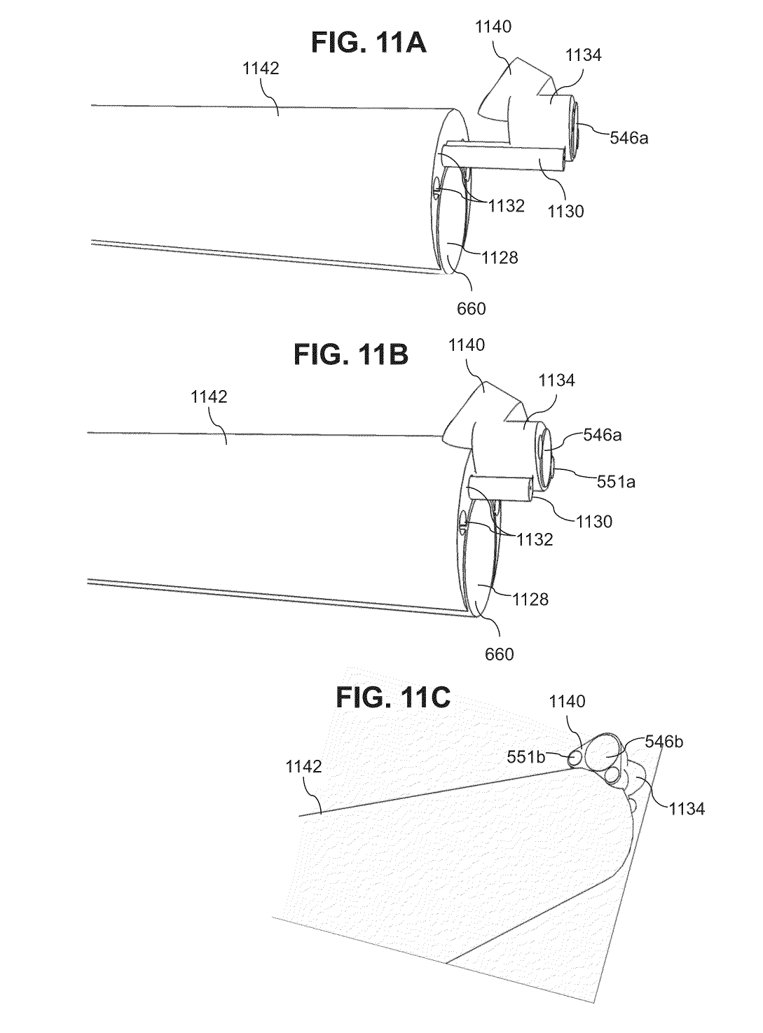

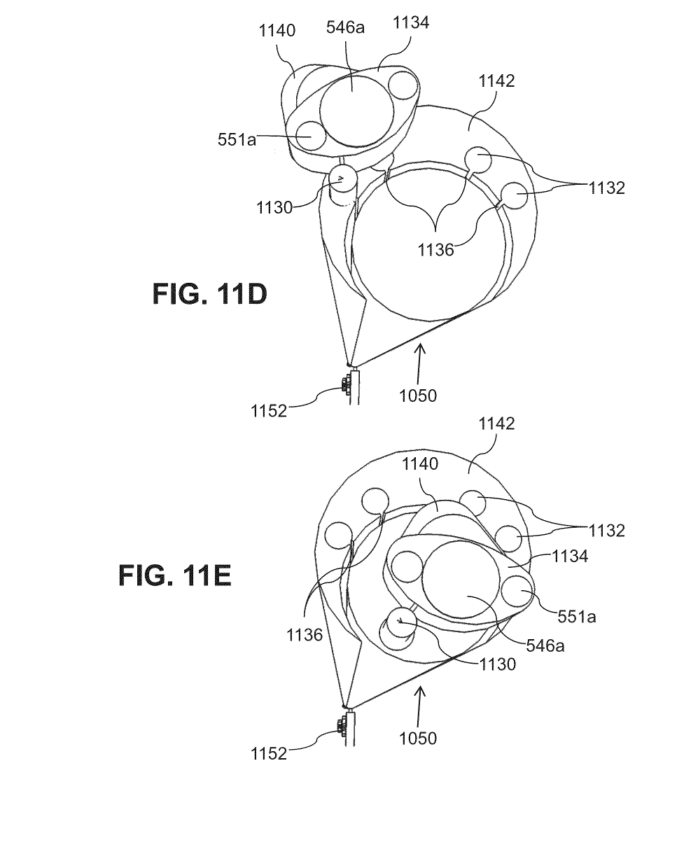

FIGS. 11A-11E illustrate a tool with a mount for support on an external surface of a catheter according to an embodiment of the present invention. Optionally a tool 1134 may include a mount 1140 for supporting the tool on the exterior surface of a catheter.

In some embodiments, tool 1134 (for example a cameras 546a,b pointed forward and backward with lighting 551a,b) may be inserted through a working channel and/or extended out the working channel and/or supported on a catheter in a manner similar for example to tool 534 illustrated in FIGS. 6A-6F. For example, a carriage 1130 may be inserted into one of a plurality of guide 1132 channel of an outer sleeve 1142. A guide channel 1132 optionally includes a slit 1136. Tool 1134 may be conveyed through working channel 1128. Optionally tool 1134 is pushed and/or controlled and/or manipulated by carriage 1130. For example tool 1134 may be conveyed along working channel 1128 tool 1134 it extends out the distal end of sleeve 1142. Optionally while tool 1134 is extended out of working channel 1128, carriage 1130 is twisted to deploy tool 1134 away from the axis of working channel as illustrated for example in FIG. 11A. Optionally, tool 1134 is retracted back until mount 1140 fits over the distal end of outer sleeve 1142 supporting tool 1134 on the outer surface of sleeve 1142. The configuration of mount 1140 allows supporting tool 1134 on the outer surface of sleeve 1142 without requiring a mount on the sleeve. The configuration of mount 1140 allows insertion of carriage 1130 through various guides 1132 of sleeve 1142 and/or supporting tool 1134 in various orientations for example as illustrated in FIG. 11D. Optionally, tool 1134 remains connected to carriage 1130 while tool 1134 is supported on the outside of the catheter. Alternatively or additionally, in some embodiments a tool may be disconnected, connected and/or reconnected with a carriage while the tool is supported on the outside of a catheter. Optionally, tool 1134 can be retrieved and redeployed from the proximal end of sleeve 1142 without removing sleeve 1142 from the target site in the patient. For example, this may make it possible to conveniently reposition a tool 1134 to get a better angle to view or act upon a target. Optionally, tool 1134 may be removed and/or repositioned depending on the status of sleeve 1142. For example, while sleeve is being moved through a narrow region, tool 1134 may be removed from sleeve 1142 to facilitate navigation in tight spots. Alternatively or additionally tool 1134 may be moved into the distal end of working channel 1128. In the distal end of working channel 1128 a camera in tool 1134 may be used to help navigate the catheter without protruding from the catheter and interfering with movement. Optionally, tool 1134 may be removed and/or replaced while sleeve 1142 remains in place in a patient. For example, a camera may be replaced with an alternative camera that has a different field of view and/or focal length and/or a different profile and/or a different sensitivity.

FIG. 11C illustrates tool 1134 stabilized on an outside surface of sleeve 1142 in accordance with an embodiment of the current invention. For example, while tool 1134 is stabilized on a catheter, lens 546b is outside the cross section of the catheter. While protruding from a distal opening of the catheter and/or while supported on the outside of the catheter, camera of tool 1134 views backwards (in the proximal direction) without being blocked by the catheter body.

FIG. 11E illustrates tool 1134 inserted into endoprobe 1050 which is inserted through sleeve 1142 in accordance with an embodiment of the current invention. Endoprobe 1050 and/or outer sleeve 1142 may optionally include a steering mechanism 1152.

### Assembling a Multi-Part Catheter

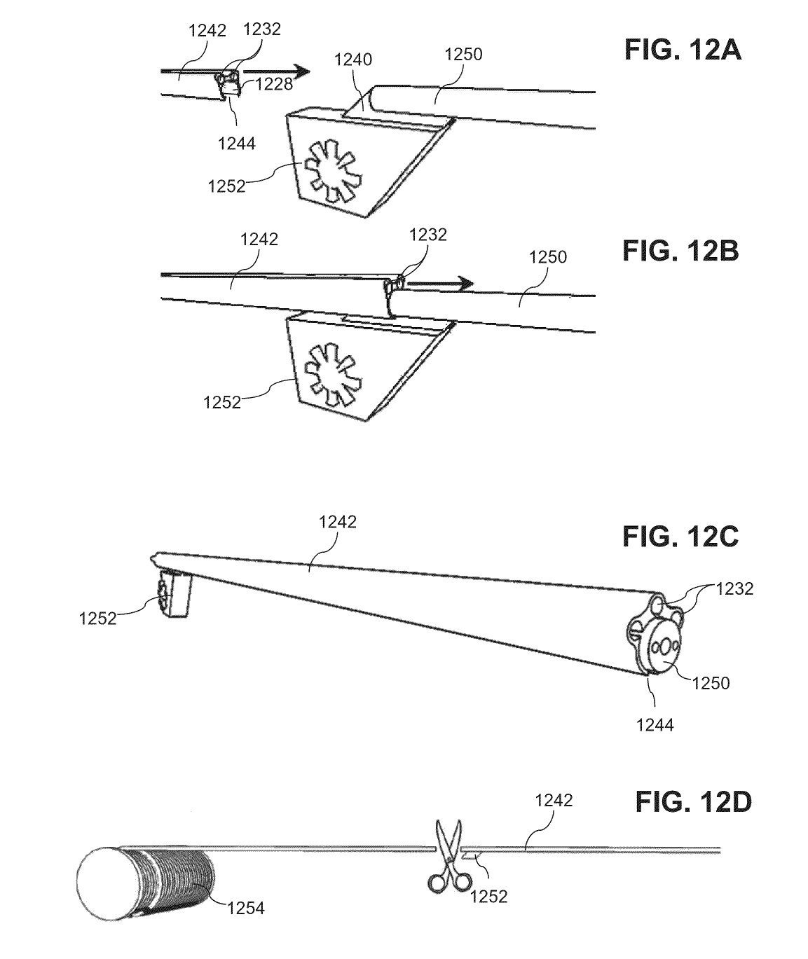

FIGS. 12A-12D illustrate positioning of an outer sleeve 1242 around an endoprobe 1250 and/or a steering mechanism 1252 in accordance with an embodiment of the present invention. Optionally, outer sleeve 1242 includes a working channel 1228 and a plurality of guides 1232. For example, working channel 1228 may include a longitudinal opening 1244 (for example see FIG. 12C).

In some embodiments, sleeve 1242 may be positioned onto endoprobe 1250 from a proximal end to endoprobe 1250 to the distal end thereof. For example, opening 1244 may fit around a mount 1240 of steering mechanism 1252. Optionally, mount 1240 fixes an orientation of sleeve 1242 with respect to endoprobe 1250. For example, endoprobe 1250 may be inserted into a subject and then sleeve 1242 may be inserted into the subject and/or over endoprobe 1250. Alternatively or additionally, sleeve 1242 may be positioned over endoprobe 1250 and the entire assembly may be inserted into the subject and/or advanced to a region of interest inside the subject. Alternatively or additionally, endoprobe 1250 may be inserted into a subject and then sleeve 1242 may be inserted into the subject and/or over endoprobe 1250 and then the entire assembly may be advanced to a region of interest inside the subject. In some embodiments, endoprobe may be removed from sleeve 1242 and working channel may be used for insertion of a tool and/or a material into the subject.

In some embodiments, a sleeve may be positioned over an endoprobe from the distal end of the endoprobe to the proximal end. Optionally, after positioning of the sleeve, the assembly may be inserted into a subject. Alternatively or additionally, the sleeve may be inserted into the subject and/or the endoprobe may be inserted into the subject through the sleeve.

In some embodiment, an outer sleeve may include a multi-channel hose. For example the hose may be disposable and/or passive. For example the hose may be supplied as a bulk roll 1254 and/or used as needed, for example as illustrated in FIG. 12D.

### Integrally Steered Catheter

FIGS. 13A-13C illustrate an integrally steered catheter assembly in accordance with an embodiment of the current invention. Optionally a sleeve 1342 includes one or more working channels 1328 and/or one or more guides 1332 and/or an integral steering mechanism 1352. Optionally, guides 1332 communicate with channel 1328 along the entire length thereof. Optionally, channel 1328 includes a longitudinal opening 1344 along the entire length thereof. Optionally guides 1332 are in the form of thin channels distributed around working channel 1328. Optionally all and/or some of the guides have a proximal opening 1359 and/or a distal opening 1358.

In some embodiments guides 1332 may communicate with channel 1328 along a portion of a length thereof. Optionally, working channel 1328 may include a longitudinal opening along a portion of the length thereof. Optionally, working channel 1328 includes a distal opening 1360 and/or a proximal opening 1361.

### Closed Multi-Track Outer Sleeve

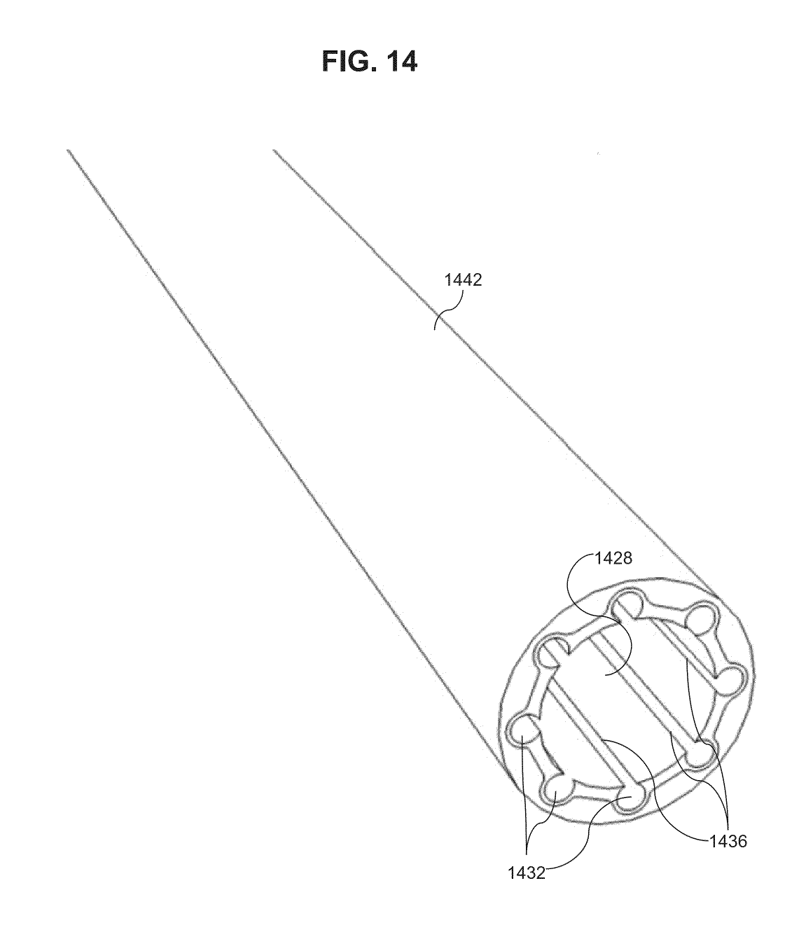

FIG. 14 illustrates a sleeve 1442 with a closed working channel 1428 in accordance with an embodiment of the current invention. A closed working channel 1428 may be useful for example when fluids need to be transported in the working channel and/or when the path to the region of interest includes delicate structures and/or delicate materials and/or dangerous materials that may be contaminated and/or cross contaminated when an open channel is inserted through them. Optionally, working channel may have a longitudinal opening near the distal end for example over a length ranging between 1 mm to 1 cm and/or 1 to 10 cm and/or 10 to 20 cm. Optionally, a working channel may have a longitudinal opening near the distal end for example over a length ranging between 1 times the width of the working channel to 10 times the width of the working channel and/or 10 to 20 times the width of the working channel and/or 20 to 100 times the width of the working channel. Also illustrated are guides 1432 and communication openings 1436.

Sharing a Working Channel for Access and/or Fluid Transport

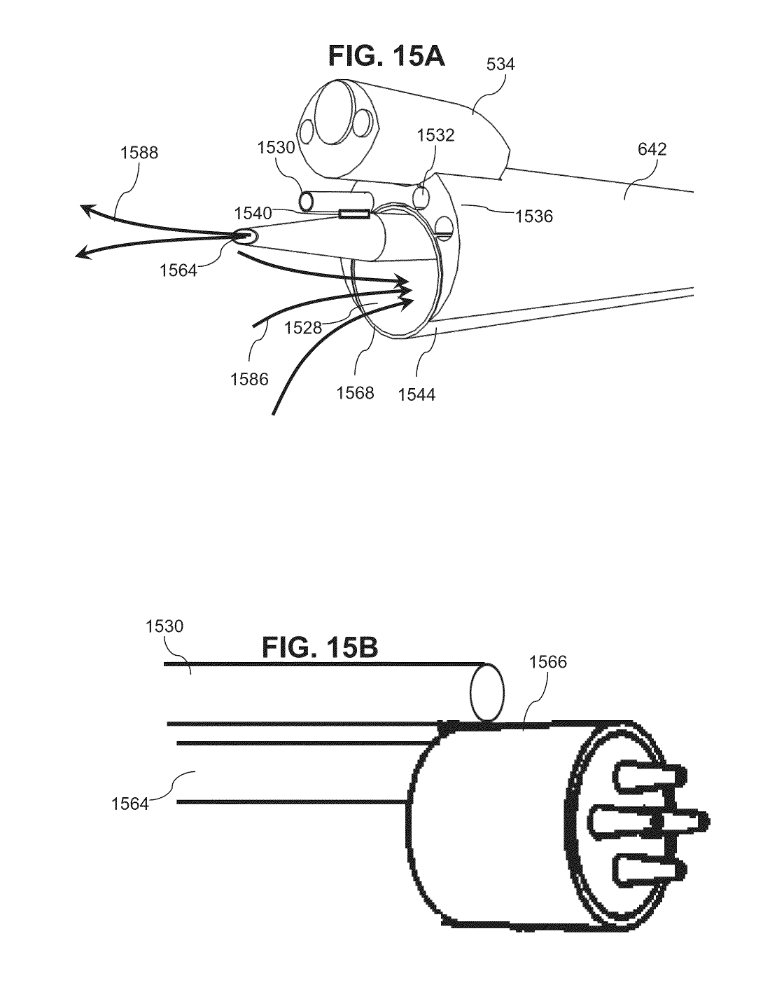

FIG. 15A illustrates use of a working channel 1528 for fluid transport in accordance with at least one embodiment of the current invention. For example fluid may be pumped into and/or suctioned 1586 out of a lumen directly through a working channel 1528. Optionally an inner sleeve 1568 is inserted into working channel 1528 to prevent fluid from leaking into a communication opening 1544. Alternatively or additionally, a tube 1564 may be inserted through a working channel. Optionally irrigation fluid is pumped 1588 into the lumen through tube 1564. Alternatively or additionally fluid may be suctioned out of a lumen using a tube. Optionally the tube 1564 may be conveyed through channel 1528 and/or controlled in the lumen using a carriage 1530 and/or guide 1532 and/or mount 1540. For example, a carriage 1530 may slide along a guide 1532. Carriage 1530 may be connected to tube 1564 by a mount 1540. For example, mount 1540 may pass through a communication opening 1536 between a guide 1532 channel and working channel 1528. Optionally sleeve 1568 may include a slit to allow communication between guide 1532 and working channel 1528. In some embodiments of the current invention, one or more tubes and/or tools may be conveyed through a working channel, deployed, controlled and/or retrieved without the use of a guide. Optionally, tube 1564 extends distally beyond the opening of channel 1528. For example, the distance between the end of tube 1564 and the opening of channel 1528 may impede diversion of fluid exiting tube 1564 by suction 1586 from channel 1528.

In some embodiments, fluid being suctioned out of a lumen may include for example blood and/or mucus and/or feces and/or pieces of tissue and/or a stone and/or a misplaced object (for example a suture that was left behind and/or a clip that was improperly placed). For example, when colonoscopy is being performed on an improperly prepared patient (due to an emergency situation and/or incomplete preparation) irrigation and/or suction can be used to clean an area. Optionally in accordance with the current invention a single catheter may be used to search the intestine and/or irrigate and/or clean an investigational area (including for example suctioning 1586 feces through a large working channel 1528). Then without moving, removing and/or replacing the catheter (for example sleeve 642) tools may be introduced into the lumen for a therapeutic procedure. Optionally, a large portion of the cross section of working channel 1528 (for example between 30 to 50% of the cross section and/or between 50 to 70% of the cross section and/or between 70 to 100% of the cross section) is available over a large portion of the length of channel 1528 (for example between 30 to 50% of the length and/or between 50 to 70% of the length and/or between 70 to 100% of the length) for various functions (such as transporting fluids and/or debris and/or tools) while a camera (for example tool 534) remains in the lumen. For example tool 534 may continuously monitor the lumen while working channel is performing the various functions. Optionally various tools and/or materials may be moved into and/or out of the lumen through working channel 1528 while sleeve 642 and/or tool 534 remains in place.

FIG. 15B illustrates an alternative washing nozzle 1566 in accordance with an embodiment of the present invention. Optionally a small tube 1564 is connected to a large spray nozzle 1566 for cleaning objects in a lumen. Nozzle 1566 is attached to a carriage 1530. For example nozzle 1566 is may be large enough to block a majority of a cross section of working channel 1528. For example, tube 1564 only blocks a small portion of working channel 1528 (for example between 5% to 25% and/or between 25% and 50%). Optionally, during washing, nozzle is supported on carriage 1530 away from the distal opening of channel 1528. During washing channel 1528 is optionally used for suctioning fluids away from the work area. Optionally, a large spray nozzle 1566 can be used for irrigation and/or washing of objects while a majority of channel 1528 is used for suctioning away debris.

### Use of Guide Channels

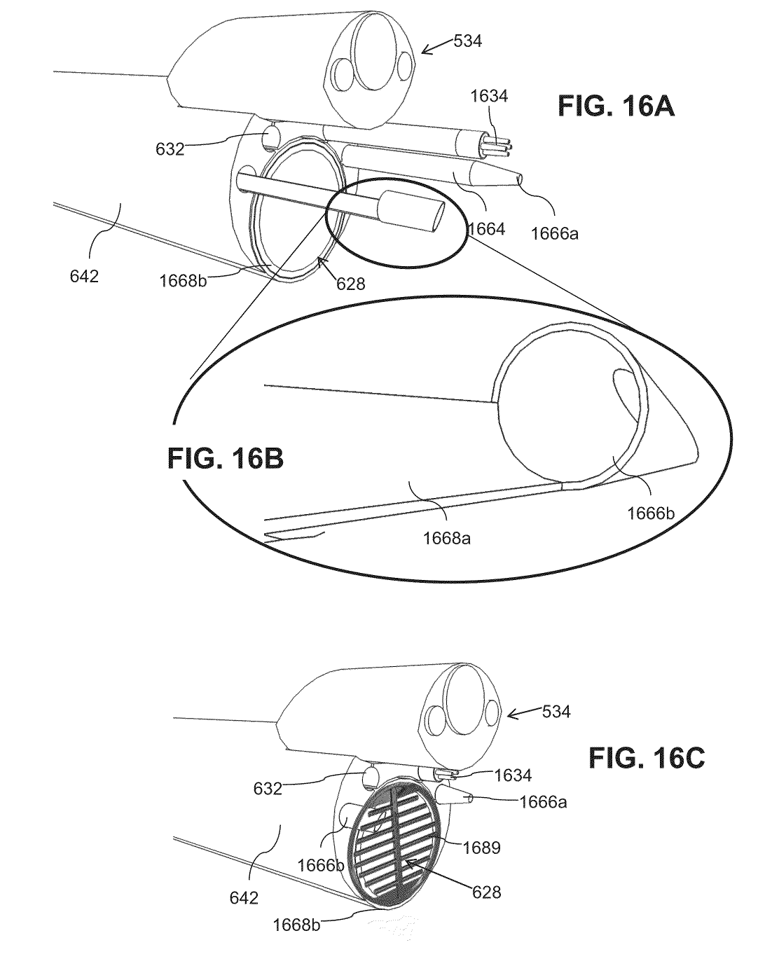

FIGS. 16A-C illustrate use of a guide 632 channel to perform a task in accordance with an embodiment of the present invention. For example, a tool 1634 may be conveyed to a region in a subject through a guide 632 channel. Alternatively or additionally a guide 632 channel may be used to convey a fluid. For example, a tube 1664 and/or nozzle 1666a may pass through a guide 632 channel and/or a sleeve 1668a and/or nozzle 1666b may a pass through a guide 632 channel. For example, sleeve 1668a may block a communication opening 636 for example to prevent fluid from leaking between working channel 628 and guide 632. Optionally sleeve 1668a may have an open cross section, blocking just communication opening 636. Optionally a sleeve 1668b may be inserted into working channel 628. For example sleeve 1668b has a closed cross section. Alternatively or additionally an open cross section sleeve may be used for example to close off longitudinal opening 644. A guide 632 channel and/or a working channel 628 may be used for example to pass a fluid into a subject and/or to drain fluid from a subject and/or suction material from the subject. Optionally, a sleeve 1668b may include a filter 1689 and/or a grating, for example to prevent clogging of channel 628 by large particle and/or objects.

Transport of Large and/or Folding Objects

FIGS. 17A-E illustrate use of a working channel with a longitudinal slit to transport a large object in accordance with an embodiment of the present invention. For example, a sample and/or a tool and/or a vessel may be larger than a working channel of a catheter. The object may be conveyed along a lumen extending out a longitudinal opening, completely and/or partially outside of a catheter. Optionally a folding vessel may be used to contain a large object.

In some embodiments, a folding sample bag 1770 be deployed and/or retrieved. Optionally bag 1770 is mounted on a carriage 1730 and/or a mount 1740. For example, bag 1770 can be conveyed inside working channel 628 and/or deployed in a folded state for example as illustrated in FIG. 17A. Once deployed, bag 1770 may open, for example as illustrated in FIG. 17B.

In some embodiments, mount 1740 may be biased to an open configuration. Optionally, inside working channel 628 the walls of the channel hold mount 1740 in the closed configuration. When mount 1740 is extended out of distal opening, it optionally springs open as illustrated for example in FIGS. 17B and 17C. Optionally mount 1740 and/or bag 1770 may be conveyed along working channel and/or deployed out of distal opening 660 by a carriage 1730. Carriage 1730 may travel along guide 632.

FIGS. 17B and 17C illustrate bag 1770 deployed in a deployed state in accordance with an embodiment of the current invention. In the deployed state, bag 1770 may optionally be manipulated using carriage 1730. For example, twisting carriage 1730 causes bag 1770 to turn over (for example as illustrated in FIG. 17C). While in the deployed state bag 1770 can be used to collect samples, for example a large polyp inside of an intestine of a subject.

FIG. 17D illustrates returning sample bag 1770 and/or the polyp into working channel 628 in accordance with an exemplary embodiment of the current invention. Optionally, returning bag 1770 to working channel 628 squeezes mount 1740 closing the opening of bag 1770. The bottom of bag 1770 extends out of longitudinal opening 644 of channel 628.

FIG. 17D illustrates conveying sample bag 1770 and/or the large polyp along working channel 628 (for example in order to retrieve the polyp) in accordance with an exemplary embodiment of the current invention. Bag 1770 is optionally conveyed along a lumen and/or path. For example bag 1770 may be conveyed by a user retracting and/or pulling on a proximal end of carriage 1730. For example, when sleeve 642 is located in a relatively large lumen (for example a lumen that has a larger cross section than sleeve 742) (for example an intestine) bag 1770 can be used to convey a relatively large sample (for example the bag and/or sample may be between as large to twice as large as the cross section of the working channel and/or between twice to five times as large as the cross section of the working channel) without crushing or breaking the sample. When the sample reaches a narrowing of the lumen (for example the end of the lumen for example the anus and/or rectum of the intestine) it is optionally detached from guide 632 and/or carriage 1730 and/or mount 1740 and/or conveyed and/or allowed to exit working channel 628. For example bag 1770 may be extracted from channel 628 out of longitudinal opening 644. Sleeve 642 and/or bag 1770 may then be retrieved independently. For example bag 1770 may be retrieved with a tool, for example forceps.

### Method of Using a Large Tool

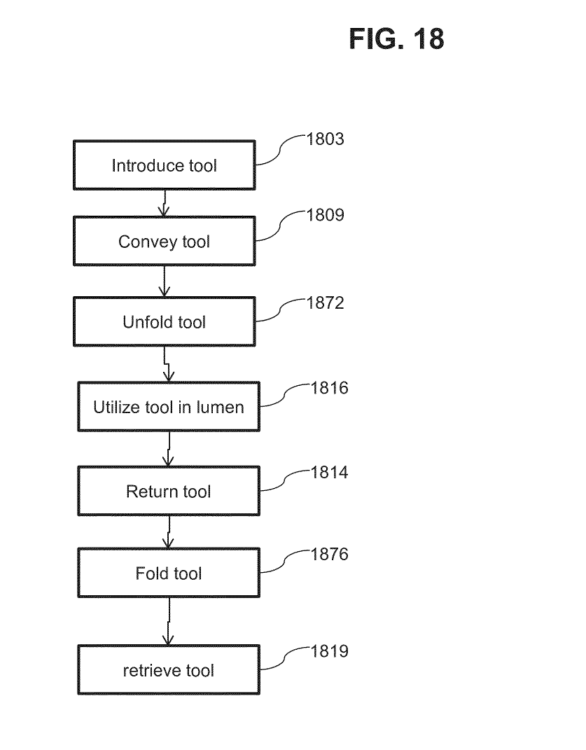

FIG. 18 illustrates a method of employing a large tool in a subject in accordance with an embodiment of the present invention. For example, a large tool is to be used at a site in a lumen of a patient. The site may be accessible by a path. For example, the access path may include a location wherein the path is narrow. The method optionally includes inserting the tool past a narrowing, and/or unfolding the tool and/or conveying the tool along the path to the site and/or using the tool and/or returning the tool. For example, the tool may include a sample collecting bag. For example the lumen may include a large intestine. For example, the narrowing may be at the opening of the lumen, for example the anus and/or rectum.

In some embodiments a tool may be introduced 1803 into a subject past a narrowing of access path to the lumen. Optionally the tool may be in a folded state when introduced 1803 past the narrowing. Alternatively or additionally a second tool may be used to introduce 1803 the first tool, for example a forceps may be used to introduce a large tool through an anus and/or a rectum.

In some embodiments, the large tool will be conveyed 1809 along the access path to the lumen. For example a guide may be used to convey 1809 the tool to the lumen. Optionally the tool may be conveyed 1809 in a folded state and/or unfolded 1872 upon arriving at the deployment site. Alternatively or additionally, the tool may be unfolded 1872 after introduction 1803 past the narrowing and/or may be conveyed 1809 in an unfolded state.

In some embodiments the tool will be utilized 1816 at the site. For example, a sample may be placed in a sample bag. While at the site the tool may, for example, be controlled using a guide and/or carriage and/or mount for example as described herein.

In some embodiments, the tool may be returned 1814 to the access pathway. Optionally the tool may be folded 1876 before return 1814 and/or retrieval 1819. Alternatively, the tool may be returned 1814 in an unfolded state. For example the tool may be conveyed along the access path in a folded and/or unfolded state. Optionally, the tool may be retrieved 1819 back out of the subject.

### States of a Folding Tool

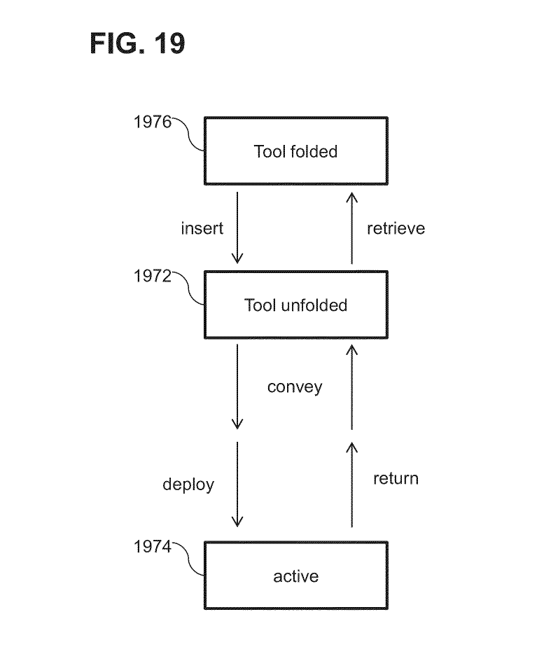

FIG. 19 is a state diagram of a folding tool, for example a sample bag in accordance with an embodiment of the current invention. Optionally, a tool may have a folded state 1976 and/or an unfolded state 1972 and/or an active state 1974.

In some embodiments, the tool may be inserted and/or retrieve through a narrowing of an access path in a folded state 1976. Alternatively or additionally the tool may be inserted and/or retrieved in an unfolded state 1972. Optionally an extraction tool, for example forceps, is used to insert or retrieve the folding tool.

In some embodiments, the folding tool may be conveyed and/or deployed to a site of interest in the body. Conveyance and/or deployment may optionally be in an unfolded state 1972. Alternatively or additionally conveyance and/or deployment may be in a folded state 1976.

In some embodiments, the tool may be employed at the site of interest in an active state 1974 state (for example for a sample bag the active state may include an open state in which objects may be placed in the bag).

In some embodiments a tool is returned to an access path and/or conveyed along the path. For example returning and/or conveying may be in an unfolded state 1972.

In some embodiments a folding tool may be returned to an access path and/or conveyed along the path without use of a guide. In some embodiments a tool larger than the cross sectional area of a working channel may be returned to a working and/or conveyed along a working channel having a longitudinal opening and/or slit without use of a guide.

### Multi-Track Catheter

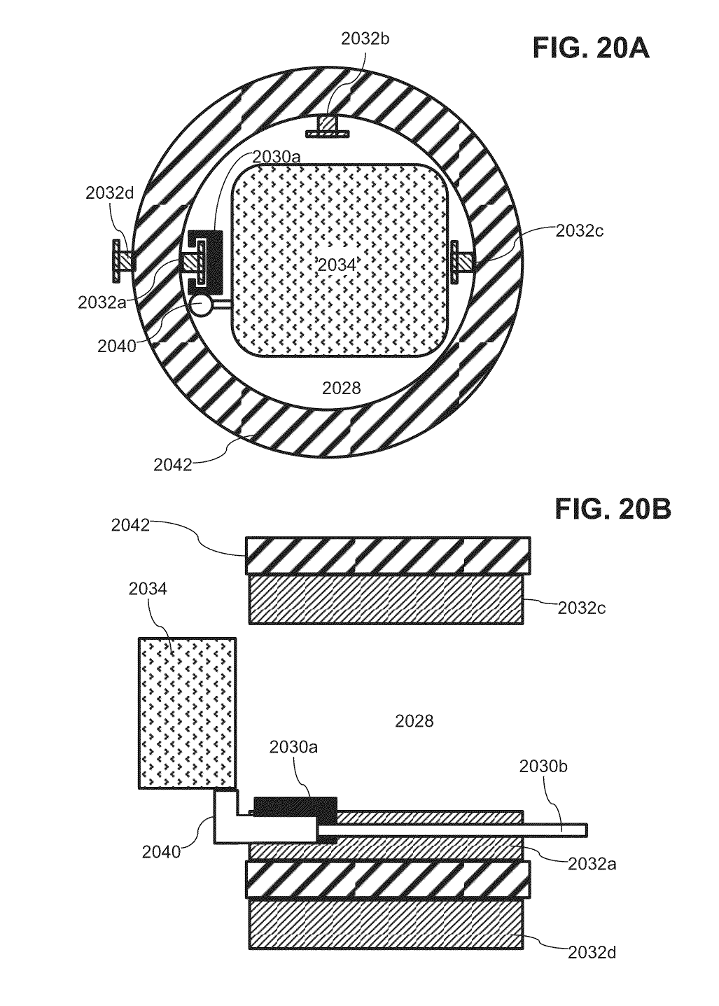

FIG. 20A is an axial cross section of a working channel 2028 having track guides and a tool in accordance with an embodiment of the current invention. Guides 2032a-c of working channel 2028 may for example include one or more tracks running longitudinally along the inside of a sleeve 2042. Alternatively or additionally, guides 2032d of a working channel 2028 may for example include one or more tracks running longitudinally along the outside of sleeve 2042.

In some embodiments, a tool 2034 may be connected to a mount 2040 and/or one or more carriages 2030a,b. For example carriages 2030a,b may include a sliding carriage 2030a. Carriage 2030a may for example slide along guide 2032a and/or convey tool 2034 along working channel 2028. When carriage 2030a is at a distal end of guide 2032a mount 2040 and/or tool 2034 may optionally extend out of working channel 2028 (for example as illustrated in the transverse cross section of FIG. 20B). Optionally, mount 2040 is supported on a hinge. For example, when tool 2034 is extended out of working channel 2028, a user twists carriage 2030b to move tool 2034 away from the axis of channel 2028.

In some embodiments, tool 2034 may fill more than 50% of the cross section of the working channel 2028. Optionally, a second tool may be introduced to channel 2028 behind tool 2034. For example, when tool 2034 is moved away from the axis of channel 2028 the second tool may be pushed out the opening of the channel.

### External Guides

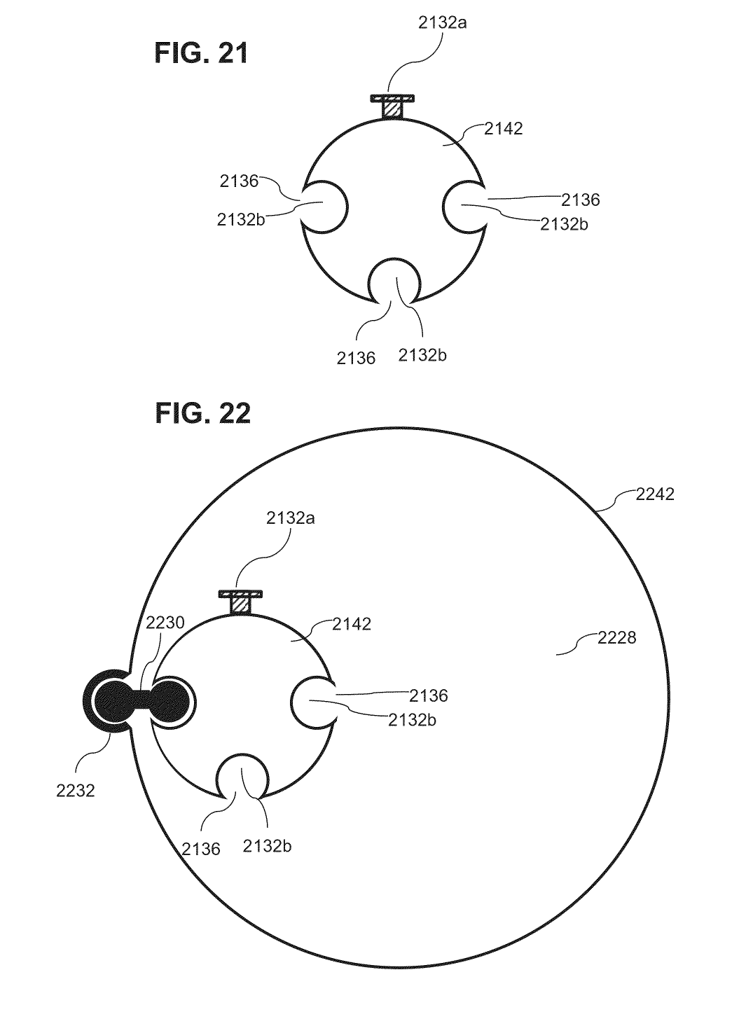

FIG. 21 illustrates a schematic cross sectional view of an endoprobe 2142 with external guides 2132a and 2132b. External guide 2132a optionally includes a track. Guides 2132b optionally include channels. Optionally guides 2132b have longitudinal slits 2136. Slits 2136 optionally communicate with the outside of endoprobe 2142. For example slits 2136 may communicate with an access path to a site of a procedure and/or a lumen of a living creature. For example, endoprobe 2142 with external guides 2132a and/or 2132b may be placed in a lumen of a living creature. For example, endoprobe 2142 with external guides 2132a and/or 2132b may be placed along an access path to a treatment site. Tools (for example tool 534 and/or tube 1564 and/or tool 934a and/or tool 2034) may be connected to guides 2132a and/or 2132b (for example via a carriage (for example carriage 530 and/or 2030a,b) and/or a mount (for example mount 540 and/or mount 2040)). The tools may be conveyed to the treatment site and/or controlled at the treatment site using guides 2132a and/or 2132b. Optionally an endoprobe may include one or more guides of a single guide and/or one or more guides of different kinds.

### External Guides in a Working Channel

FIG. 22 illustrates endoprobe 2142 with external guides 2132a,b inserted through a working channel 2228 of a larger endoprobe 2242 in accordance with an embodiment of the current invention. For example the external guides 2132a,b may be used for conveying tools and/or materials through working channel 2228. Optionally a carriage 2230 may link a guide 2232 of large endoprobe 2242 to a guide 2132b of endoprobe 2142. Alternatively or additionally, the outer endoprobe may be a simple catheter with no guides and an endoprobe with external guides (for example endoprobe 2142) may be inserted inside and used to convey multiple tools and/or materials through the larger catheter.

### Supporting a Tool on Multiple Carriages

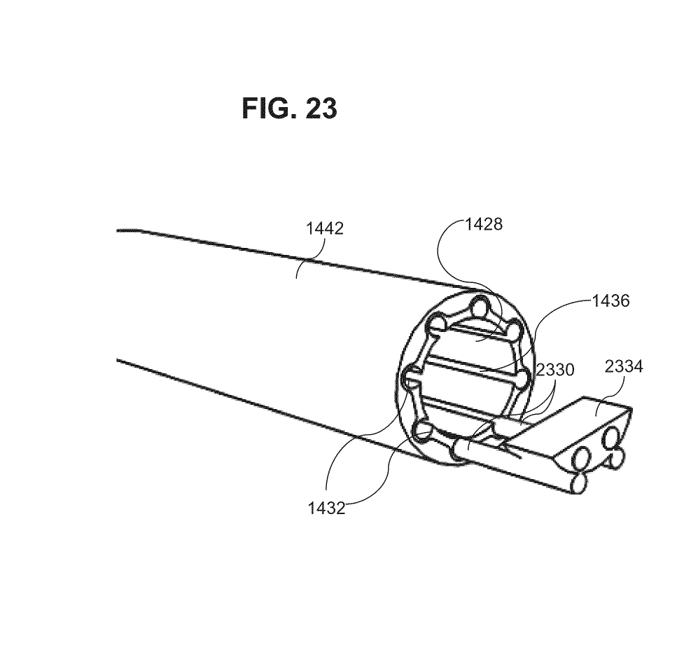

FIG. 23 illustrates a tool 2334 connected to multiple carriages 2330 and/or guides 1432 in accordance with an embodiment of the present invention. For example, tool 2334 may be conveyed through channel 1428 and/or supported in a lumen by multiple carriages 2330. Extra support supplied by multiple carriages may be useful for example for heavy tools and/or tools that need to be extra steady. In some embodiments, a tool may be conveyed through at least a portion of a working channel by a single guide and/or carriage and then connected to a further guide and/or carriage for deployment. Alternatively or additionally, a tool may be conveyed by the multiple carriages and/or disconnected from one or more of the carriages during deployment.

### Rounded Guide Tracks

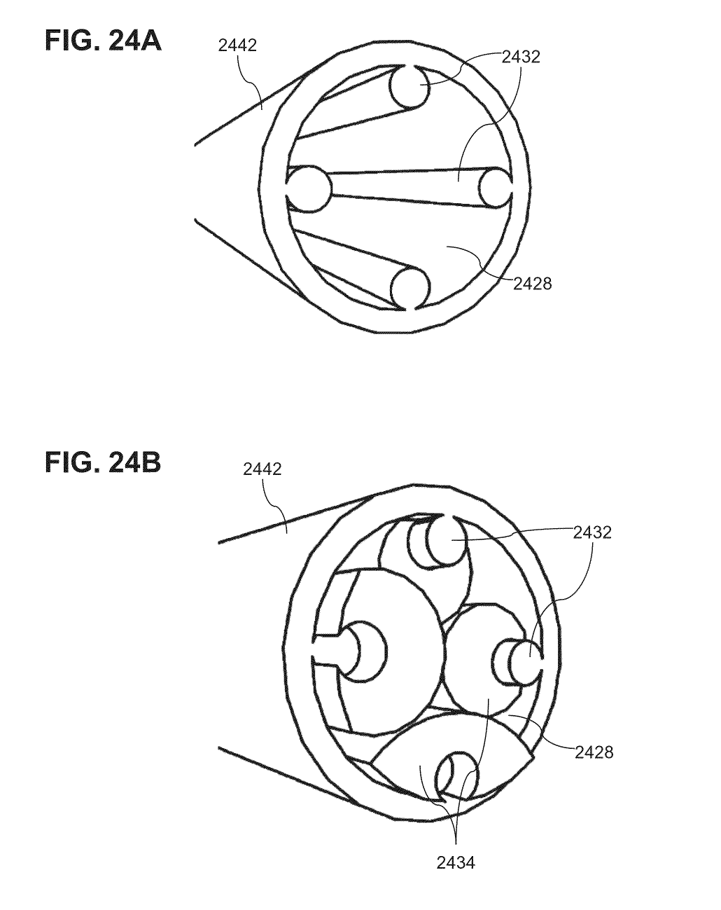

FIGS. 24A, B illustrate rounded guide 2432 tracks in accordance with an embodiment of the present invention. For example sleeve 2442 includes guide 2432 tracks distributed around the inner perimeter of a working channel 2428. Alternatively or additionally, an endoprobe may include rounded guide tracks on an external surface.

FIG. 24B illustrates four tools 2434 connected to guide 2432 tracks in accordance with an embodiment of the current invention. Optionally sleeve 2442 may be integrally molded with guide 2432 tracks and/or tracks may be attached to a sleeve. Optionally an endoprobe with an external guide channel (for example catheter illustrated in FIG. 21) may be connected to guides 2432, for example for conveying the endoprobe through sleeve 2442. For example, a single guide 2432 on the inside and/or outside of a catheter can be connected to the endoprobe of FIG. 21 to increase the number and/or type of available guides. Optionally sleeve 2442 may be a passive sleeve. Alternatively or additionally, sleeve 2442 may include an integral steering mechanism. In some embodiments a sleeve with rounded tracks may include a working channel with a longitudinal opening.

### T-Shaped Guide Slots

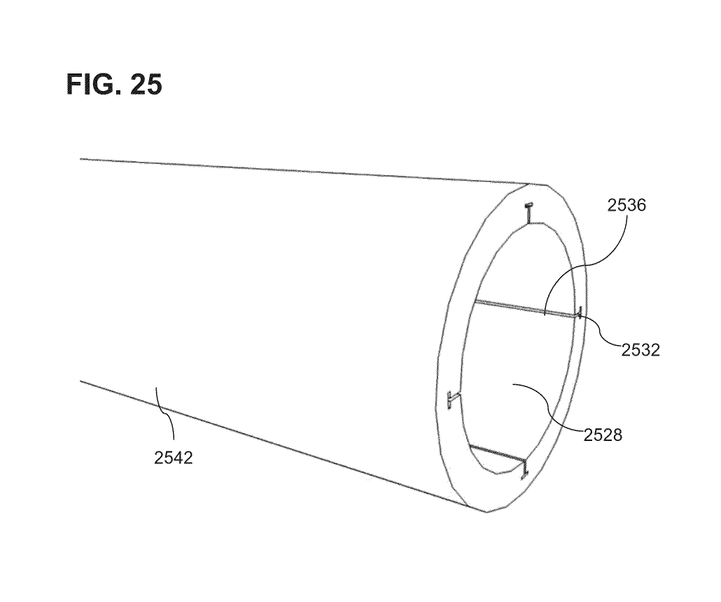

FIG. 25 illustrates a T-shaped slot guide 2532 in accordance with an embodiment of the present invention. Optionally, a T-shaped slot guide 2532 communicates with a working channel 2528 of sleeve 2542 via slits 2536. Optionally, slits 2536 may be open only along the entire length of guides 2532. Alternatively or additionally, slits 2536 may be open only along a portion of a guide 2532. T-shaped guides 2532 may stabilize the tool from rotation. In some embodiments a single sleeve may have various kinds of guides on the inside and/or outside of the sleeve.

### Application of a Multi-Function Catheter

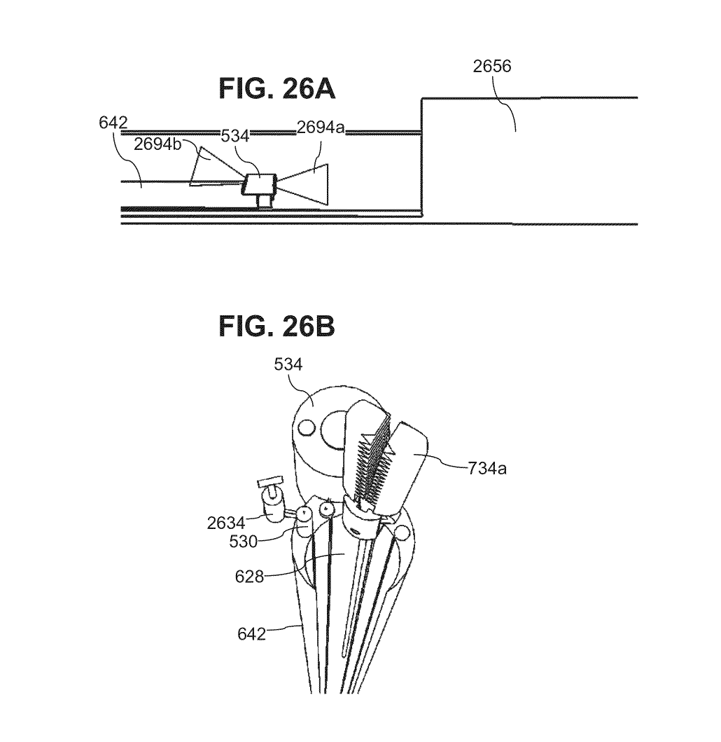

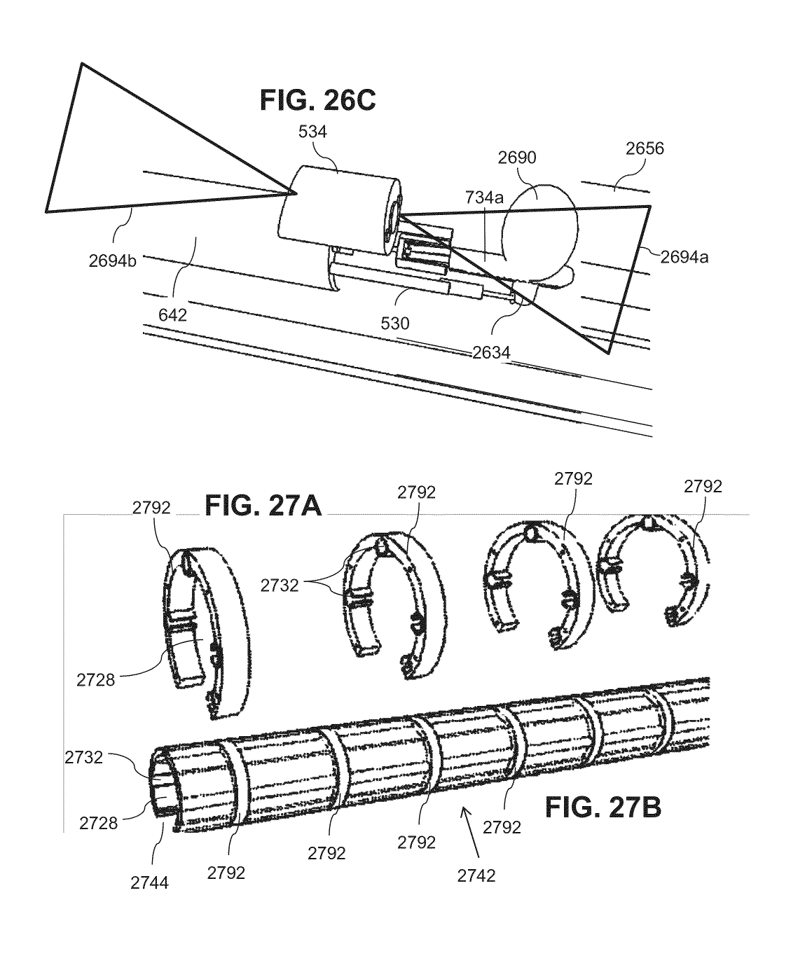

FIGS. 26A-C are perspective views of a catheter in use for removing a growth (for example a polyp) in accordance with an embodiment of the current invention. In the example various tools are applied over time to detect a health condition and/or intervene with a treatment.

FIG. 26A illustrates a multi-function catheter in a search mode in accordance with some embodiments of the current invention. For example, the search mode may be used for detection of a disease within a lumen 2656 (for example an intestine). Optionally, camera tool 534 is mounted on a catheter sleeve 642. Tool 534 is mounted off the axis of the catheter with a field of vision 2694a distal to the catheter (looking forward) and a field of vision 2694b looking backward (from the distal end of the catheter proximally). In some embodiments, sleeve 642 may be inserted into the lumen in the configuration of FIG. 26A. Optionally, while the catheter is inserted into the lumen the camera may be used to search for symptoms. Alternatively or additionally, tool 534 may be positioned coaxially with the catheter (for example as illustrated in FIG. 6C) reducing the profile of the catheter. Alternatively or additionally, tool 534 may be located in the distal end of working channel looking ahead (out the distal end of the channel) while the catheter is being inserted. Optionally, a guide wire and/or camera may first be placed into lumen 2656 and then sleeve 642 may be inserted over the guide wire.

In some embodiments, positioning a tool 534 outside the axis of a catheter (as depicted for example in FIGS. 6F, 11B and/or 26A-C) may be useful in searching for symptoms in a lumen. For example, a camera that can see in both directions may allow searching the lumen while inserting the catheter and/or while removing the catheter. A camera with a field of vision 2694a,b may allow view both sides of an object (for example as the camera approaches an object the object is viewed from one side with the one field of vision 2694a,b and/or after the catheter passes by the object, the other side of the object is seen with the other field of vision 2694b,a. For example, a catheter may be inserted beyond a region of interest and the drawn back while searching for a condition requiring treatment. Tool 534 may optionally include a long focal length camera looking backward and/or proximally (for example field of vision 2694b). The backwards field of vision 2694b may be used for spotting suspicious objects. Optionally, the forward field of vision 2994a may be used for spotting suspicious objects from the opposite side. Depending on the location of abnormal structures, obstructions etc. an object may be more easily spotted from one side. Additionally or alternatively, the trailing field of vision (for example the forward field of vision 2994a when searching is performed while removing the catheter and/or the rearward field of vision 2994b when searching is being done as the catheter is inserted) may have a smaller focal length than leading field of vision and/or may be used for investing objects closely and/or during an intervention.

In some embodiments, when the distal end of the catheter passes a suspicious object spotted previously in the rear facing field of vision 2694b, the forward field of view 2694a may be used for investigating the suspicious object and/or for directing intervention targeting the object. Optionally, a camera used for investing objects and/or directing intervention may have a smaller focal length and/or narrower field of view than a camera used for spotting objects. Alternatively or additionally, once a suspicious object is spotted, further sensors may be deployed for diagnosing the object. For example, the further sensors may include a camera with specific frequency band sensitivity (for example for spectral analysis) and/or having higher resolution and/or a shorter focal length and/or a temperature sensor and/or a chemical sensor etc. Deployment of further sensors may optionally be made while the catheter and/or the originally camera of tool 534 remains deployed (thus keeping sight of the object and avoiding loosing contact with the object while deploying the new sensor).

FIG. 26B illustrates a catheter in a treatment mode in accordance with some embodiments of the current invention. Optionally, a catheter is switched from the search mode to the treatment mode when a condition is found that requires treatment. A catheter may have multiple search and/or treatment modes that may be selected during a medical procedure and/or without removing the catheter from the patient. Optionally, to change modes, tools are introduced and/or removed and/or replaced while the distal end of the catheter remains in the lumen and/or the treatment site and/or sensors (for example in tool 534) keep track of the object. In the exemplary embodiment of FIG. 26B, a forceps (for example tool 734a) and a cutter (for example tool 2634) are deployed for an intervention.

FIG. 26C illustrates an exemplary intervention in accordance with an embodiment of the current invention. For example, a polyp 2690 may be grasped with forceps tool 734a and/or cut off with cutter 2634. Guides (for example 632) and/or carriages (for example 530) are optionally used to control tools 2634 and/or 734a to perform the procedure. Optionally a sample bag (for example bag 1770) may be deployed and/or used to retrieve polyp 2690. When necessary a tool can be retrieved and redeployed in a different guide for improved access to a treatment site. In some embodiments, further diagnostic and/or treatment tools may be deployed to while the catheter remains in place and while tracking the suspicious object. After a procedure is completed tools 734a and/or 2634 are optionally retrieved and/or new tools are optionally deployed for a further procedure and/or search mode is restarted. Retrieving and/or deploying tools and/or changing modes are optionally carried out while the distal end of sleeve 642 remains in the lumen and/or the site of the procedure. Optionally after the procedure, a search for suspicious objects is resumed. Optionally, as further objects are identified and/or diagnosed further treatment and/or investigative and/or diagnostic tools may be deployed using the same catheter which optionally remains in the lumen.

### Stiffening Rings

FIGS. 27A, B illustrate stiffeners in accordance with an embodiment of the present invention. Optionally a catheter and/or a sleeve (for example sleeve 2742) may include a structure (for example rings 2792). The structure may for example preserve the geometry of a working channel 2728 and or a guide channel 2732 as the catheter snakes around inside a lumen. For example the stiffeners may keep guide channel 2728 open and/or keep a longitudinal opening 2744 open and/or keep a guide channel 2732 open.

### Viewing Tools

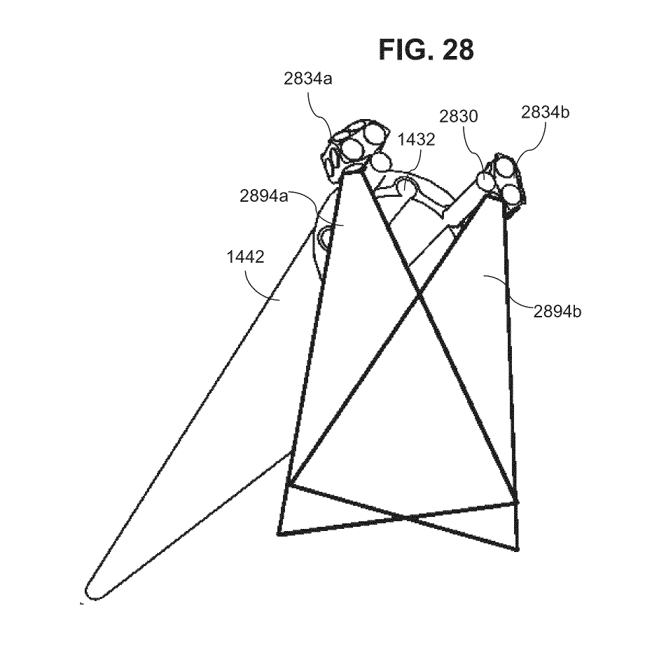

FIG. 28 illustrates further options for sensing and/or viewing tools in accordance with an embodiment of the present invention. In some embodiments, tools 2834a,b may be deployed to sense, diagnose, illuminate and/or view targets at various angles and/or in various frequency bands and/or in cooperation. For example, two imaging sensors with overlapping fields of view 2894a,b may be used cooperatively to produce stereoscopic vision. Alternatively or additionally, a single sensor may be inserted, manipulated, retrieved and/or reinserted to view an object from various viewpoints. Control of tools 2834a,b may optionally be achieved using guides 1432 and/or carriages 2830. Alternatively or additionally, different sensors may be deployed, retrieved and/or redeployed for different tasks in a lumen while the catheter and/or other tools and/or other sensors continue operation. In some embodiments, multiple sensors may be deployed from a catheter during a treatment procedure for example allowing viewing of the procedure from multiple angles.

### Forward Deployed Tools

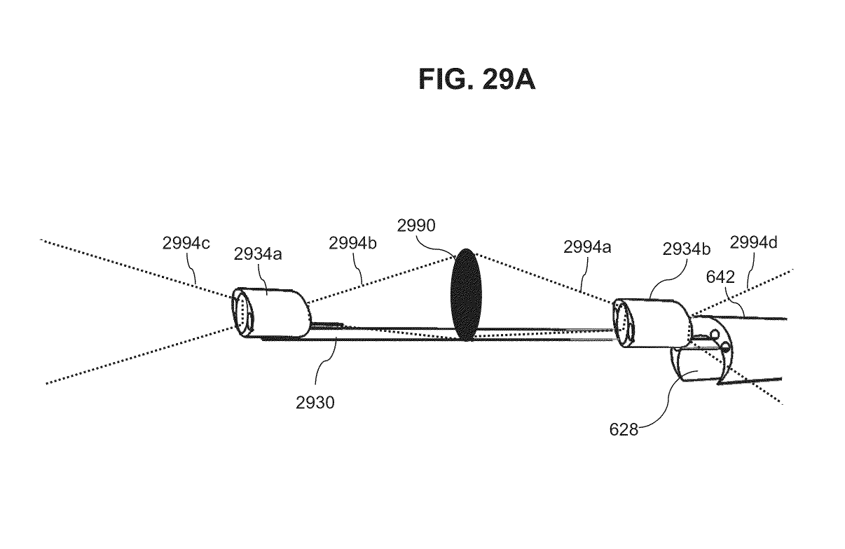

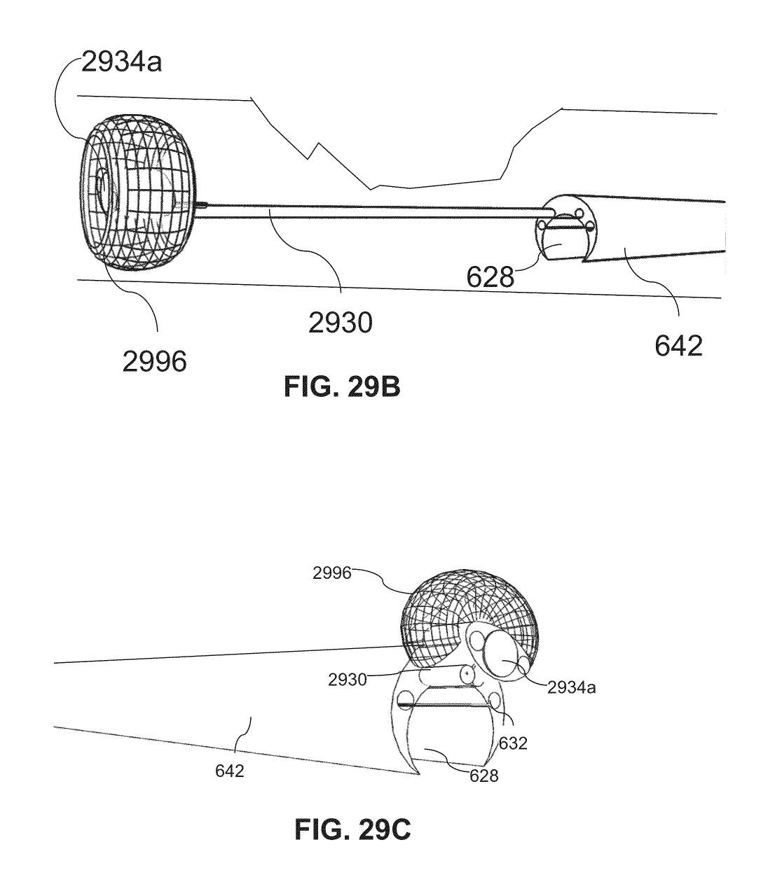

FIG. 29A illustrates a forward deployed tool 2934a in accordance with some embodiments of the current invention. In some embodiments, a tool 2934a and/or a sensor may be deployed far ahead of a distal end of a catheter. For example, a viewing sensor and/or a camera may be deployed on a carriage 2930 far ahead of a catheter and/or sleeve 642 and/or an endoscope. Optionally, forward deployment may give an improved perspective when searching for structures and/or symptoms of illness. Alternatively or additionally, forward deployment may facilitate viewing an object and/or procedure from opposite sides (for example a sensor tool 2934b deployed near the catheter may view the proximal side of the procedure and/or object (for example object 2990 in field of view 2994a) and/or a forward deployed camera tool 2934a may view the distal side of the procedure and/or object (for example object 2990 in field of view 2994b)). Alternatively or additionally, the opposite views may be supplied by a single sensor at different locations and times. For example the sensor may be moved to different positions without moving the catheter and/or without interrupting the procedure. Optionally, when performing a therapeutic and/or diagnostic procedure a one or more tool and/or cameras may be deployed on opposite sides of an object. Overlapping fields of view (for example forward fields of view 2994c and 2994a or rearward fields of view 2994b and 2994d) are optionally used for binocular vision and/or for close up and wide angle views and/or for views in different spectral bands (for detecting and/or identifying and/or diagnosing various objects and/or structures).

FIGS. 29B, C illustrate a tool having a protective shield 2996 in accordance with an embodiment of the current invention. Optionally a protective shield 2996 is supplied to a tool 2934a. For example shield 2996 may protect the walls of a lumen from damage by a tool. For example a shield may include a cushion, a balloon and/or a large blunt surface. Optionally, shield 2996 may be expandable. For example, tool 2934a may be conveyed into or out of a lumen through working channel 628. While tool 2934a is passing through channel 628, shield 2996 may optionally be in a contracted state. When tool 2934a is deployed in a lumen, shield 2996 may optionally be expanded. For example, when tool 2934a is deployed forward of sleeve 642 (for example as illustrated in FIG. 29B), shield 2996 may protect the walls of an intestine from being punctured by tool 2934a. Alternatively or additionally, when tool 2934a is supported on an outer surface of sleeve 642 (for example as illustrated in FIG. 29C), shield 2996 may protect the walls of an intestine from being scraped by tool 2934a. Alternatively or additionally, a cushion and/or a shield may protect a fragile tool. Optionally shield 2996 may be expanded and/or contracted by inflation and/or deflation.

### External Guides

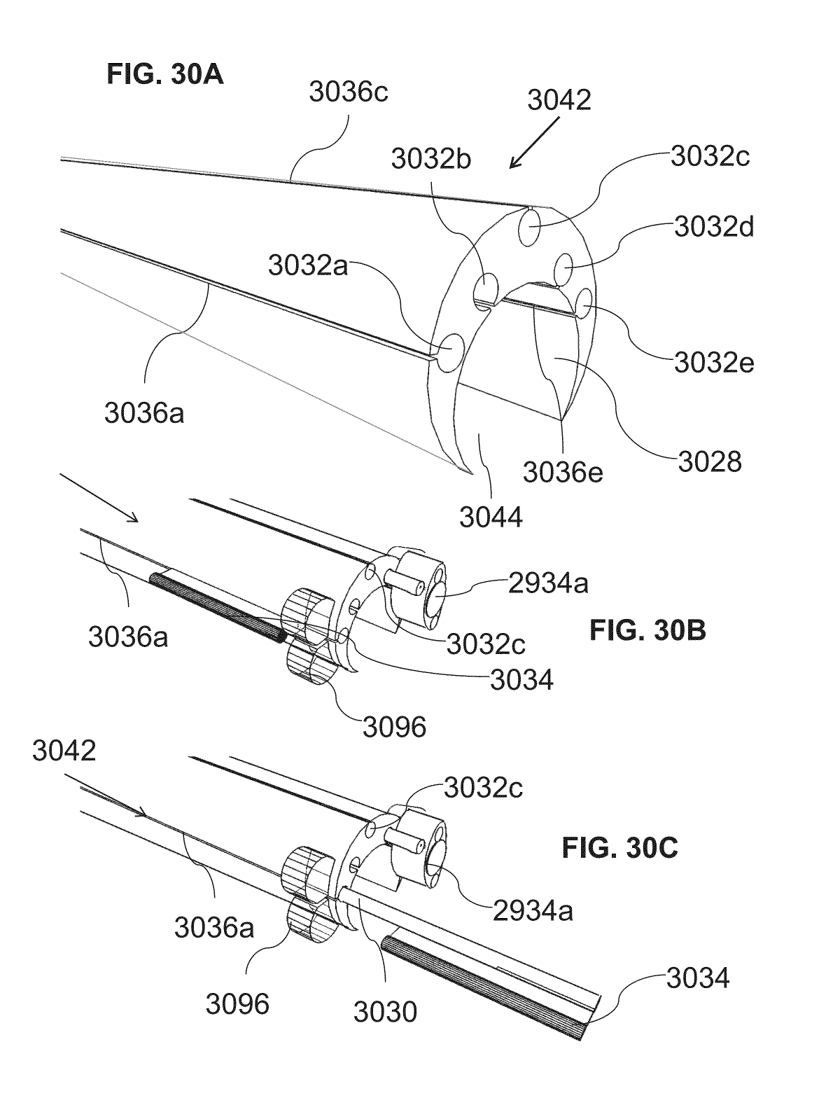

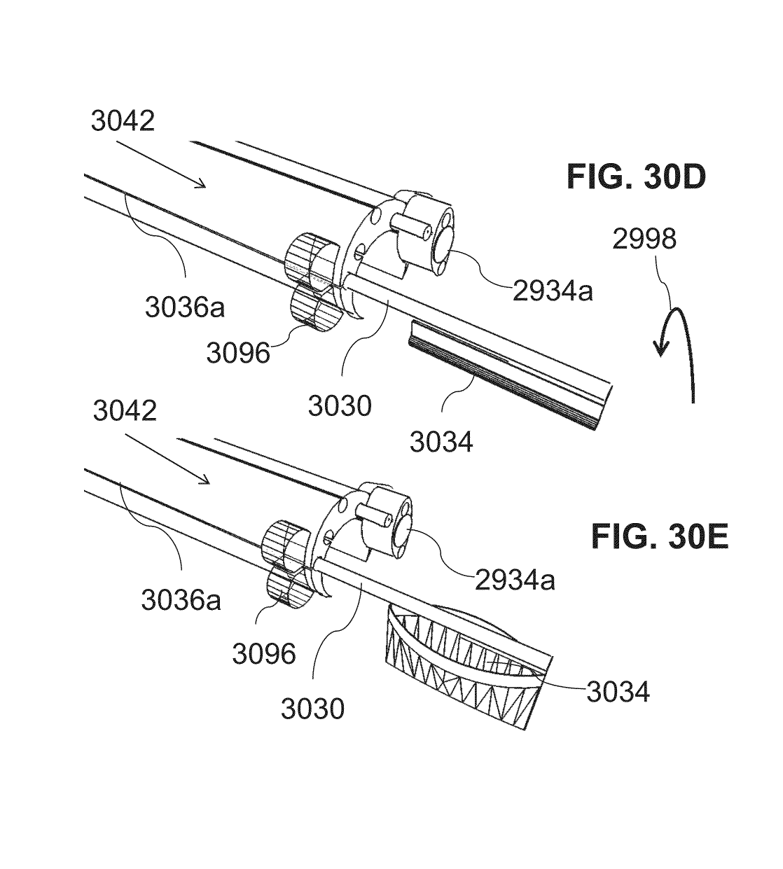

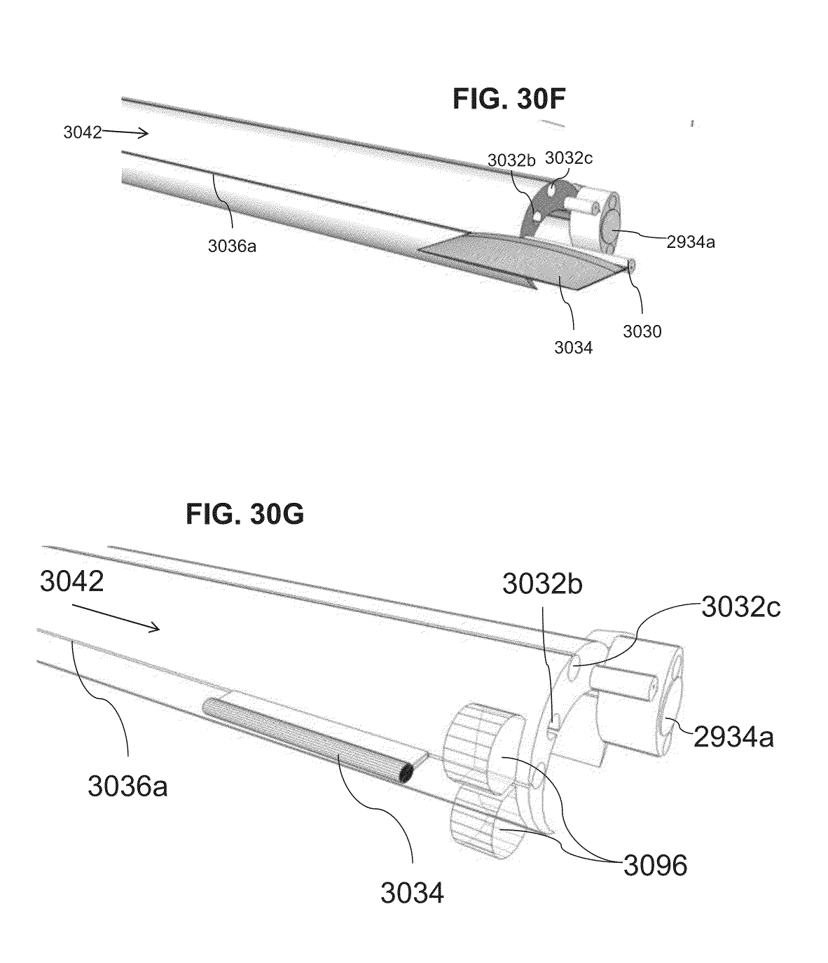

FIGS. 30A-G illustrate a catheter having an external guide in accordance with an embodiment of the current invention. Optionally an external guide may communicate with an external surface of a catheter. For example an external guide channel may include a longitudinal slit communicating with the outside of the catheter. For example, an external guide may be used to convey a large load into and/or out of a lumen. For example a large load may include a tool and/or a sample and/or a combination thereof that is larger than the working channel of the catheter and/or that does not fit through the working channel. An external guide may be used to convey a tool and/or a sensor to a location along the path of a catheter without moving the catheter itself. For example if a catheter gets hung up and/or caught and/or stuck and/or twisted and/or kinked a tool or sensor may be moved to the location of the problem. An external guide may optionally serve as an emergency conduit to remove tools from a lumen for example when the working channel of a catheter becomes blocked. Alternatively or additionally, an external guide may be used to guide a catheter. For example a first guide wire or the like may be positioned in a lumen and used to guide a catheter and/or a sleeve along the external guide to a site of a procedure. Alternatively or additionally, an external guide may be used to convey a second catheter to a site of a first catheter.

In some embodiments a catheter may include various kinds of guides. For example sleeve 3042 includes two external guides 3032a and 3032c including longitudinal slits 3036a,c communicating with an external surface of the catheter. Alternatively or additionally, the catheter may include internal guides 3032b,d,e which may communicate with a working channel 3028 of the catheter (for example by a longitudinal slit 3036e) and/or may be completely and/or partially closed. Optionally, working channel 3028 of sleeve 3042 includes a longitudinal slit 3044. Alternatively or additionally a catheter may include an external guide and/or a closed working channel.

FIGS. 30B-E illustrate a deployment of a folding sample bag 3034 using an external guide 3032a and/or carriage 3030 in accordance with an embodiment of the current invention. For example, bag 3034 may be conveyed to a site of a procedure in a folded state (for example as illustrated in FIGS. 30A-E). Bag 3034 may optionally be deployed in an open state and/or opened once deployed (for example as illustrated in FIGS. 30D, E). Bag 3034 is optionally rotated 2998 to a desired orientation.

In some embodiments a shield 3096 may be attached to a catheter near an external track. Optionally optional shield 3096 may be collapsible and/or inflatable. Alternatively or additionally, a shield may be attached to a tool, for example the bag 3034 and/or the supporting carriage 3030 of bag 3034.

FIG. 30F illustrates retrieving a sample bag 3034 with a large sample using an external guide 3032a in accordance with an embodiment of the current invention. For example the large sample may be retrieved along the outside of the catheter.

FIG. 30G illustrates an optional shield 3096 in accordance with an embodiment of the current invention. For example, bag 3034 may be inserted through a narrow entrance to a lumen (for example through an anus and/or rectum leading to a colon) in a folded and/or unshielded state (for example with shield 3096) in a contracted state. Once in the lumen (for example the colon), shield 3096 may be expanded. Optionally bag 3034 is conveyed along external guide 3032a to a site of a procedure in a folded state with shield 3096 is an expanded state (for example protecting the walls of the colon from being scratched by bag 3034). Optionally, bag 3034 is deployed at the site of the procedure and/or opened and/or returned along the outside of sleeve 3042 with a large sample.

### Alternate Cross Sections

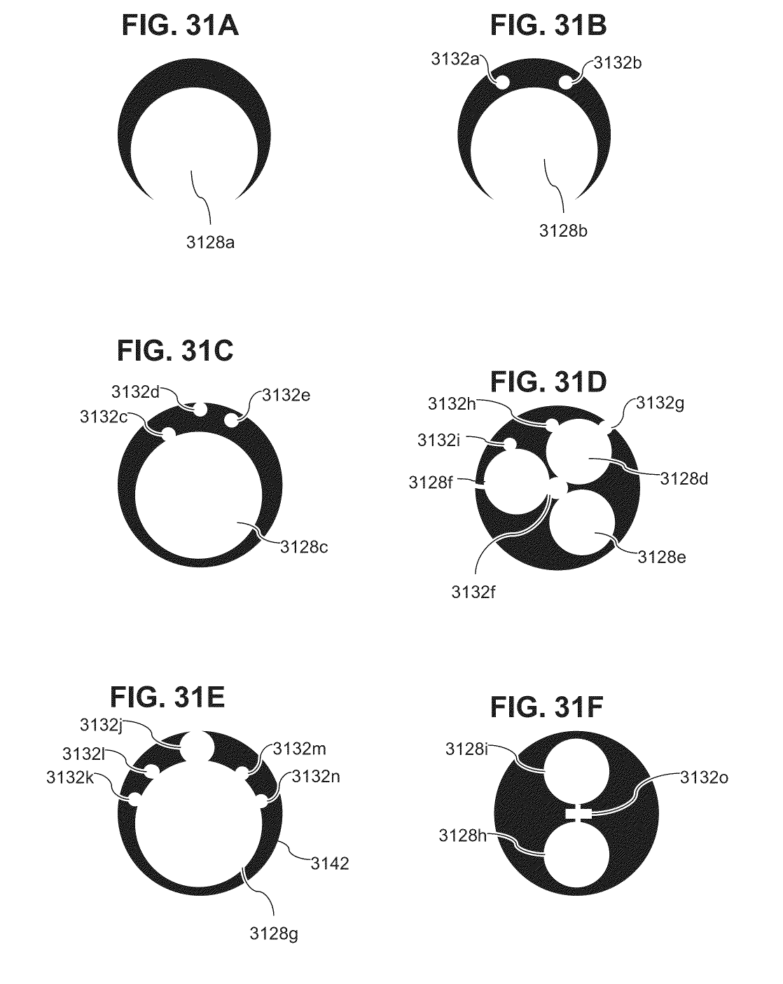

FIGS. 31A-F illustrate various optional configurations for working channels and/or connecting channels in accordance with some embodiments of the current invention. In the spirit of the current invention a catheter may include and/or not include one or more working channels, internal guides (for example communicating with one or more working channels), external guides (for example communicating with an external surface of the catheter), and/or closed guides. Optionally, a working channel may include a longitudinal opening and/or may be closed. A connecting channel may interconnect two channels (for example working channels and/or guide channels) and/or may connect a channel to an external surface of the catheter.

FIG. 31A illustrates a working channel 3128a with a longitudinal opening and no guide channels in accordance with some embodiments of the current invention.

FIG. 31B illustrates an off center closed working channel 3128b and/or closed guide channels 3132a,b in accordance with some embodiments of the current invention.

FIG. 31C illustrates an off center closed working channel 3128c and/or an internal guide channel 3132c and/or and external guide channel 3132d and/or a closed guide channels 3132e in accordance with some embodiments of the current invention.

FIG. 31D illustrates a geometry of interconnected channels in accordance with some embodiments of the current invention. For example, two or more working channels 3128d,e,f may be interconnected by a single connecting guide channel 3132f. Optionally a tool can be conveyed into a lumen through one of working channels 3128d-f using a carriage running along guide channel 3132f. The tool may optionally be deployed into a lumen at a distal end of the catheter. Optionally, once inside the lumen the carriage can be twisted around (for example similar to that twisting deployment of carriage 530 and tool 534 between FIGS. 6D and 6E. For example, the tool may be retrieved through a different one of channels 3128d-f. For example a folded sample bag could be inserted through channel 3128e and retrieved with a large sample through channel 3128d partially protruding through guide channel 3132f (for example similarly to how bag 1770 protrudes through opening 644 in FIG. 17E) from the lumen in which it was inserted. In some embodiments, when there is no carriage in guide channel 3132f, channels 3128d-f may communicate via guide channel 3132f. Optionally, a carriage may have a large enough cross section to fill channel 3132f. When the large carriage is inserted into guide channel 3132f it may optionally cut off communication between channels 3128d-f. Alternatively or additionally, a carriage may have a geometry that allows communication between some or all of channels 3128d-f at least over part of their length with the carriage is inserted into guide channel 3132f.

In some embodiments, a working channel 3128d may be connected to an external surface of a catheter via a connecting guide channel 3132g. Optionally, when no carriage is inserted into guide channel 3132g then working channel 3128d has a longitudinal slit (for example guide channel 3132g). Optionally, when a carriage is inserted into guide channel 3132g, working channel 3128d is separated from the outside of the catheter along all and/or part of its length. Alternatively or additionally, a carriage may have a geometry that allows communication between channels 3128d and the exterior of the catheter at least over part of its length with the carriage is inserted into guide channel 3132g.

In some embodiments, a working channel (for example channels 3128d,f) may be connected to one or more connecting guide channels (for example guide channels 3132f,g) and/or to one or more private guide channels (for example channels 3132i,h) that are not connected to another channel and/or lumen.

FIG. 31E illustrates a catheter cross section with a large working channel 3128g channel and/or a longitudinal connecting guide channel 3132j opening to an external surface of the catheter and/or four private guide channels 3132k-n in accordance with an embodiment of the current invention. For example sleeve 3142 may be functionally equivalent to sleeve 1142 when there is no carriage in guide channel 3132j. For example sleeve 3142 may be functionally equivalent to sleeve 1442 when there is a carriage in guide channel 3132j.

In some embodiments, a connecting guide channel may have a non-circular cross section. For example in FIG. 31F, illustrates a T-shaped connecting guide channel 3132o connecting between working channels 3128i and 3128h.

### Distal Fingers

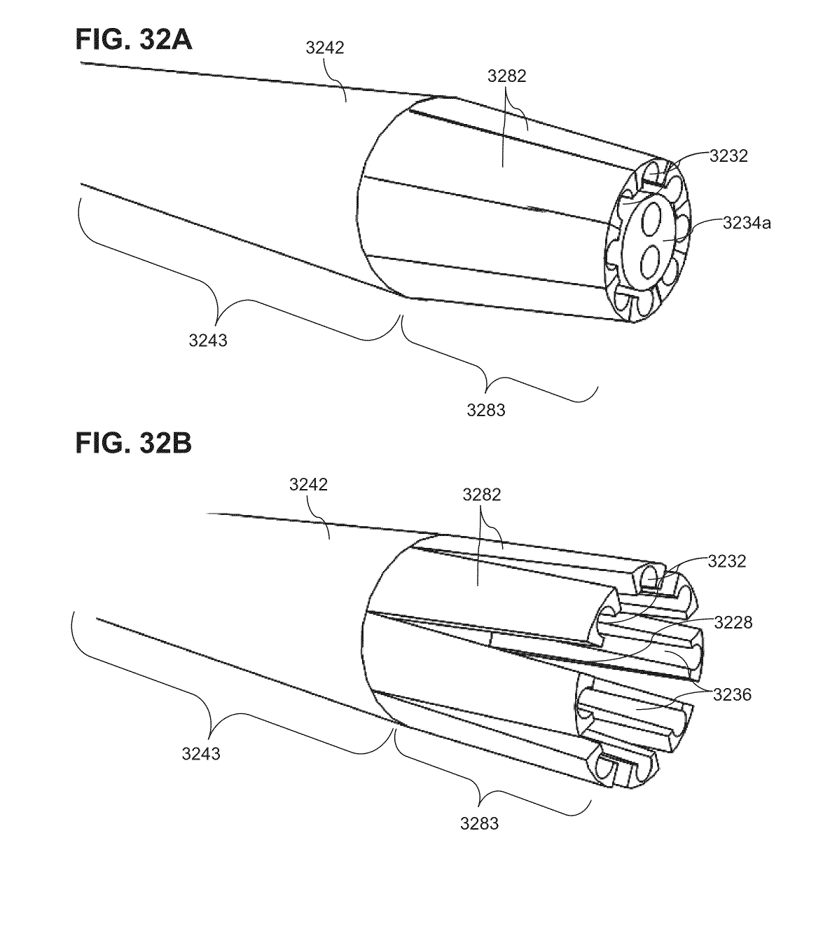

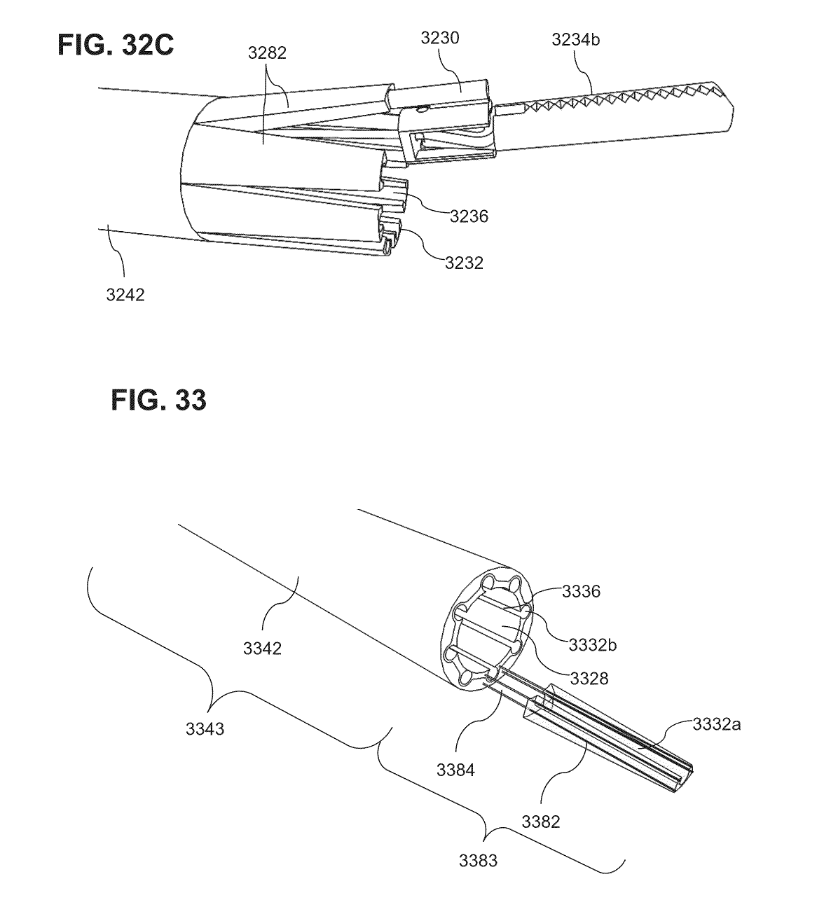

FIGS. 32A-C illustrate an endoprobe (for example sleeve 3242) with distal fingers 3282 in accordance with an embodiment of the present invention. Optionally, fingers 3282 may be pointed independently from the distal end of the sleeve. For example in FIG. 32A fingers 3282 are shown pointed inward and for example in FIG. 32B fingers 3282 are shown pointing outward from the axis of a working channel 3228 and/or a catheter and/or an endoprobe. In some embodiments, each finger 3282 may be independently pointable. In some embodiments, all or some fingers may point in a synchronized and/or simultaneous manner. For a finger may include a guide and/or a channel. By directing the fingers, an operator may optionally direct multiple channels at a distal end of a catheter. The fingers and/or channels may be directed independently and/or in a synchronized manner.

In some embodiments, the distal end of sleeve 3242 may be surrounded by fingers 3282. In some embodiments, surrounding a sleeve with fingers 3282 and/or pointing fingers 3282 inward may make it easier to maneuver sleeve 3242 in a lumen. Pointing fingers 3282 in a synchronized manner may for example stabilize and/or direct a distal end of a tool 3234a located in working channel 3228. Pointing fingers 3282 in a synchronized manner may for example help facilitate steering of a distal end of sleeve 3242 in a lumen. Movement of fingers 3282 may facilitate releasing sleeve 3242 and/or an associated tool from an entanglement.

FIG. 32C illustrates manipulating a tool 3234b in a lumen using fingers 3282 and/or a carriage 3230 in accordance with an embodiment of the present invention. In some embodiments a finger 3282 may include a guide 3232. For example a guide 3232 may continue from the main region 3243 of sleeve 3242 to the fingered region 3283. Optionally, carriage 3230 is runs along and/or passes through a guide 3232 from the proximal end of sleeve 3242 to the distal end of a finger 3282. Optionally, tool 3234b, which is mounted on carriage 3230, is pointed and/or directed in a lumen by pointing finger 3282.

In some embodiments an endoprobe may have one or a few fingers, different fingers on a single sleeve may have different geometries and/or a single finger may include multiple guides. Optionally a guide in a finger may be continuous with a guide in a main region of a catheter. Alternatively or additionally a guide in a finger may be separate from a guide in a main region of a catheter. In some embodiments a guide may be on an inner surface of a finger. In some embodiments a guide may be on a surface between two fingers. In some embodiments a guide may be on an outer surface of a finger. Optionally an endoprobe without a working channel may include fingers. Optionally a finger may include one or more guide tracks. In some embodiments a finger may be used to direct material (for example a fluid) to a site inside a living creature. For example, fluid may pass through a channel and/or a tube in the body of the catheter to the finger. For example the fluid may pass through a channel and/or a tube in the finger to a distal end of the finger and/or out a distal opening of the finger and/or to the site.

### Finger Control Mechanism

FIG. 33 is an exploded view of a sleeve 3342 including a single distal finger 3382 in accordance with an embodiment of the present invention. Optionally pulleys and/or cables 3384 may control movement of finger 3382. For example cables 3384 may pass along sleeve 3342 to a distal opening. A user at the proximal end of sleeve 3342 may control tension on cables 3384 to point finger 3382 in one or another direction.

In some embodiments a guide 3332a may be continuous from a main region 3343 of sleeve 3342 to a distal end of finger 3382. Alternatively or additionally a guide 3332b may end at the end of the main region 3343 of sleeve 3342. In some embodiments guide 3332a of a finger may communicate with a working channel 3328 of a catheter over all and/or part of its length. For example, a finger may communicate with a working channel 3328 over finger region 3383. For example a guide may include a communication slit 3336. Alternatively or additionally a guide and/or a portion of a guide on a finger may communicate with a lumen and/or an external surface and/or a guide channel of a catheter.

### Finger Filling a Slit

FIG. 34 illustrates a finger 3482 fitting a slit 3444 in a distal end of an endoscope 3442 in accordance with an embodiment of the current invention. For example, the distal end of endoscope 3442 and/or a working channel 3428 may include a slit 3444 and/or a longitudinal opening in which one or more fingers are located. For example a user may point finger 3482 outward to open the slit or point finger 3482 inward to close the slit. Alternatively or additionally, a finger may open and/or close a slit from a different direction and/or a slit may be larger than the fingers. Alternatively or additionally a finger may fit into an indentation.

### Frame for Catheter

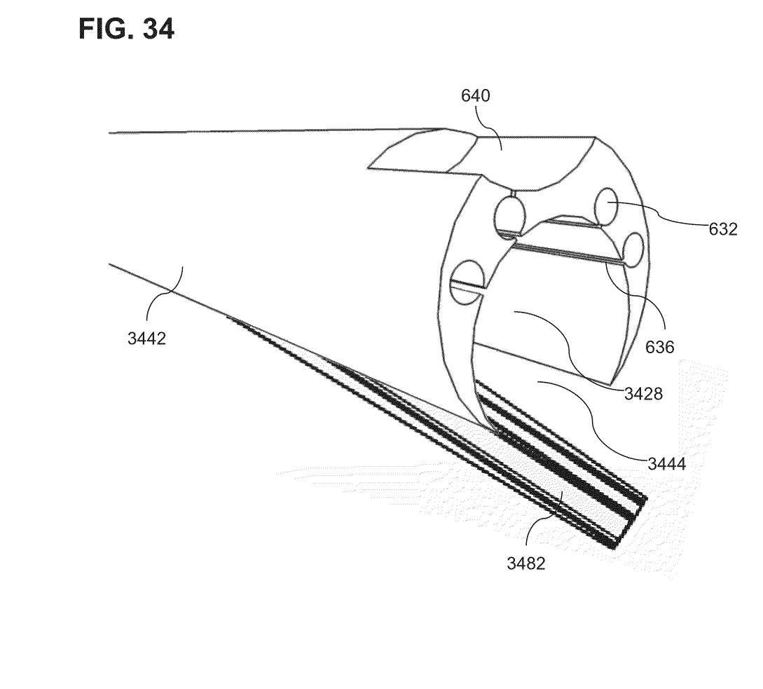

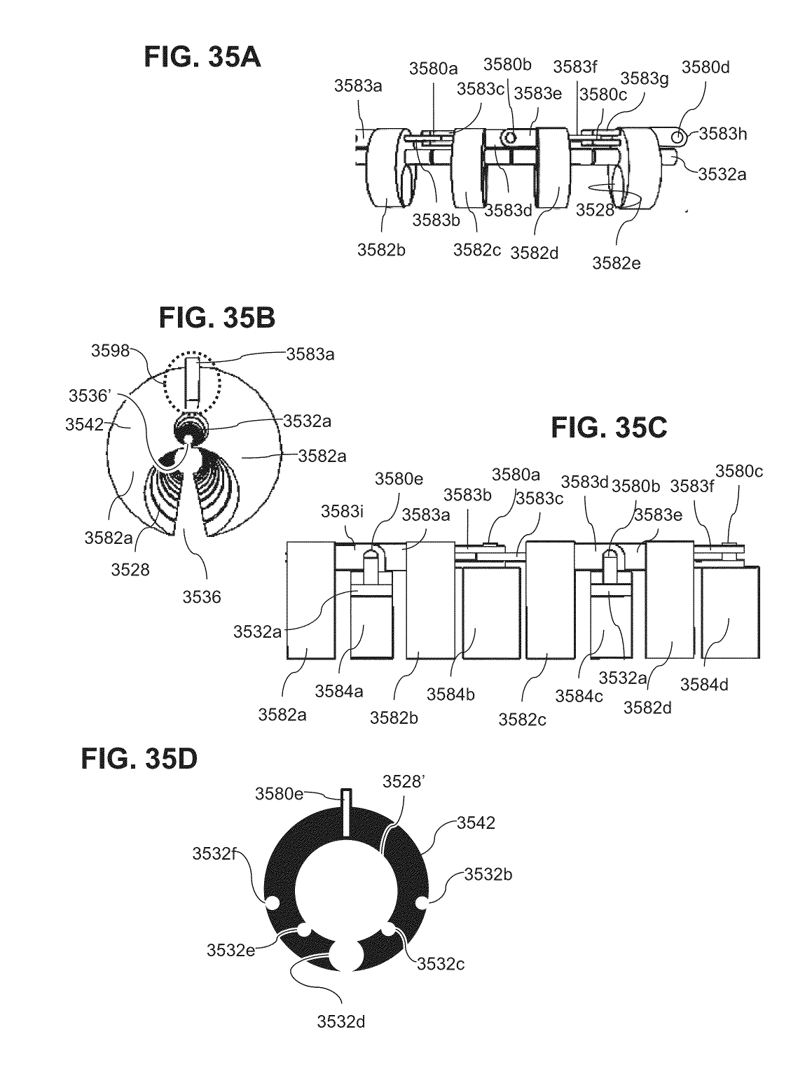

FIGS. 35A-D illustrate frames for a catheter in accordance with some embodiments of the present invention. In some embodiments, a frame may include a spine directed along one edge of the catheter. The spine may be made up of joints and/or spacers. Elements of the frame may act a ribs connected to the spinal spacers and/or joints. The spacers may, for example, define the distance between elements in the region of the spacers. Elements of the frame may preserve the shape of the channels of the catheter (for example preventing the channels from collapsing). Optionally, the spine of the frame may occupy a region along a single edge of the catheter. Limiting the cross section of the spine may allow for a variety of locations for channels along the cross section of the catheter. Channels may communicate with each other and/or with the outside of the catheter. For example a longitudinal slit may join a one channel to another channel and/or a channel to the outside of the catheter.

FIG. 35A illustrates a side perspective view of a catheter 3542 including a frame with a single spine in accordance with an embodiment of the present invention. In some embodiments, the spine of a frame may be made up of a series spacers 3583a-h joined by joints 3580a-d. Elements 3582b-e are optionally connected to the spacers 3583a-h and/or the joints 3580a-d.

In some embodiments, joints 3580a-d may include hinges which vary in orientation. The series of element may be bent in any direction by bending a combination of vertically oriented and horizontally oriented joints. For example, joints may be oriented in an alternating pattern. For example, joint 3580b, which is oriented to bend vertically around a horizontal axis intervenes for example between spacers 3583e and 3583d. Joint 3580a which is oriented to bend horizontally around a vertical axis intervenes for example between spacers 3583b and 3583c. Alternatively or additionally, some joints may bend in one direction (for example right) while others bend in the opposite direction (for example left). Optionally the end of the catheter (for example spacer 3583a) is raised by bending joints 1480b and/or 3580d. Optionally the end of the catheter (for example \ 3583a) is twisted rightward by bending joints 1480a and/or 3580c. Alternatively or additionally, joints may include ball and socket joints and/or flexible material and/or other joints. Optionally the length of the catheter and/or the spacing between elements 3582b-e may be fixed by the spine. For example bending of the catheter may be achieved by rotating elements 3582b-e around an axis on the spine. For example, the distance between elements 3582b-e in the region of the spine is optionally defined by the spacers. The distance between elements 3582b-e in the area not in the region of the spine is optionally adjusted according to steering of the catheter. For example, pulling a lower steering cable may cause the catheter to bend downward. For example, pulling the lower steering cable reduces the distance between elements at the bottom of the catheter while the distance between elements at the top of the catheter is preserved by the spacers of the spine (located at the center top of the catheter). For example, pulling a right steering cable may cause the catheter to bend rightward. For example, pulling the right steering cable reduces the distance between elements at the right side of the catheter while the distance between elements at the center of the catheter is preserved by the spacers of the spine (located at the center top of the catheter).

Optionally a frame may run the entire length of a catheter. For example, a frame may prevent collapse of channels, prevent twisting and/or limit turning all along the catheter. Alternatively or additionally, the frame may be located only near the distal end of the catheter. For example the frame may be located in distal region of length less than half of the catheter and/or less than ⅛ of the catheter and/or less than 1/16 of the catheter and/or less than 1/32 of the catheter and/or less than 1/64 of the catheter. For example the length of the framed region of the catheter may be less than 2 cm and/or less than 4 cm and/or less than 8 cm and/or less than 37 cm and/or less than 32 cm and/or less than 64 cm.

In some embodiments, the frame may assist steering of the endoscope. Alternatively or additionally the frame may prevent collapse of channels, prevent twisting and/or limit turning along all and/or part of the endoscope, for example as the endoscope is maneuvered along a lumen. Maneuvering of the endoscope may be for example by steering cables and/or by actuators for example as described herein below and/or by any means known in the art.

FIG. 35B illustrates a cross sectional view of a frame of a catheter in accordance with an embodiment of the current invention. In some embodiments, the spine of the frame may run along one edge of a catheter. For example in FIGS. 35A-C the spine is made up of joints 3580a-d which run along top (dorsal) edge of the catheter. Optionally the spine may be contained within a continuous convex region 3598 including for example less than 1/10 of the cross sectional area of the catheter. Alternatively or additionally the spine may be contained in a region of less than 1/64 of the cross sectional area of the catheter and/or 1/50 of the cross sectional area of the catheter and/or 1/36 of the cross sectional area of the catheter and/or 1/25 of the cross sectional area of the catheter and/or 1/16 of the cross sectional area of the catheter and/or 1/9 of the cross sectional area of the catheter and/or ¼ of the cross sectional area of the catheter.

In some embodiments, the cross section of the catheter includes a working channel 3528. Optionally the working channel may include a longitudinal slit 3536 communicating with the outside of the catheter. Alternatively or additionally, a working channel may communicate through a second slit 3536′ with a smaller guide channel 3532a.

FIG. 35C illustrates a catheter frame with limiting inserts 3584a-d intervening between elements 3582a-e. For example insert 3584a limits downward twisting of element 3582a (for example as element 3582a twists downward insert 3584a intervenes between the bottoms of elements 3582a and 3582b, preventing joint 3580e from bending too far). For example insert 3584b limits sideways twisting of element 3582a (for example as element 3582b twists rightward insert 3584a intervenes between the right sides of elements 3582b and 3582c, preventing joint 3580a from bending too far). The minimum radius of curvature of bending of the catheter depends on the ratios of the dimensions of the parts of the frame. For example for a longer length of joint 3580a compared to the width (along the length of the catheter) of elements 3582a, 3582b and insert 3584a, the joint will be able to rotate more sharply

FIG. 35D illustrates an alternative cross section of a catheter with a frame in accordance with an embodiment of the present invention. In the embodiment of FIG. 35D, the frame has a spine running along the top of the catheter including a joint 3580e. The cross section optionally includes a large working channel 3528′ and multiple smaller guide channels 3532b-f. For example guide channel 3532d has longitudinal slits communication with outside the catheter and also with working channel 3528′. For example guide channels 3532e, 3532c have longitudinal slits communicating with working channel 3528′. For example guide channels 3532b, 3532f include longitudinal slits communicating with the outer surface of the catheter.

### Actuators for Steering an Endoscope

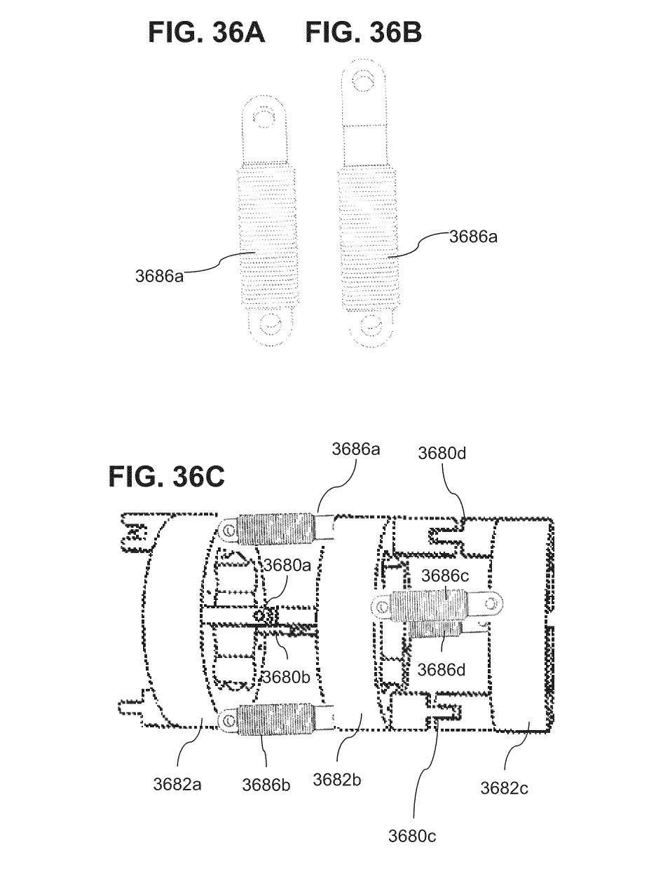

FIGS. 36A-C illustrate the use of actuators for steering of an endoscope in accordance with an embodiment of the current invention. Actuators optionally allow independent control of various sections of an endoscope. In some embodiments, independent control may facilitate more precise steering of the endoscope than, for example, steering cables.

FIGS. 36A, B illustrate an exemplary magnetic steering actuator 3686a in a contracted and extended configuration respectively in accordance with an embodiment of the current invention. An operator optionally uses a remote control to contract or extend actuators inside a lumen of a patient.

FIG. 36C illustrates an endoscope frame made up of elements 3682a-c joined by joints 3680a-d and actuators 3686a-d in accordance with an embodiment of the present invention. Optionally, by selectively extending and/or contracting actuators 3686a-d an endoscope can be steered. For example to steer the end element 3682a upwards actuator 3686a would be contracted while actuator 3686b would be extended. For example to steer the end element 3682a rightward actuator 3686d would be contracted while actuator 3686c would be extended. For example, each actuator may be one or more wires running to a power source outside the endoscope. Each actuator may be separately controlled by supplying a current to its respected wire. Optionally a single wire may be connected to more than one actuator. Alternatively or additionally, a controller may be located inside the endoscope. The controller may control individual actuator according to control signals received from outside the endoscope. Use of a controller optionally reduces the number of wires running all the way through the endoscope.

### Immobilizing an Endoscope

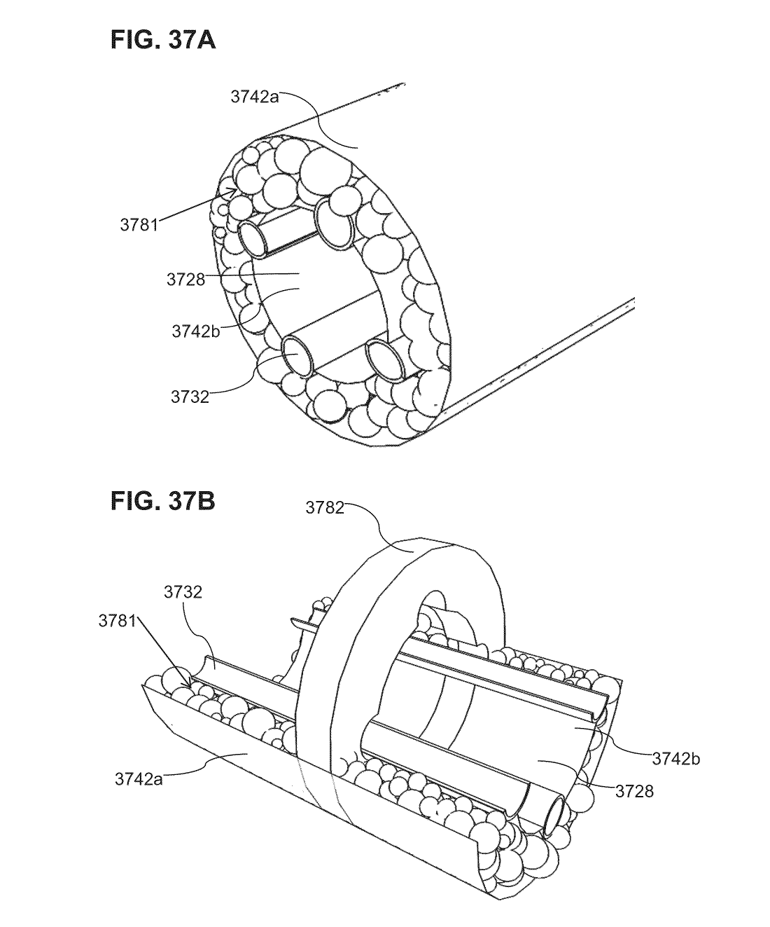

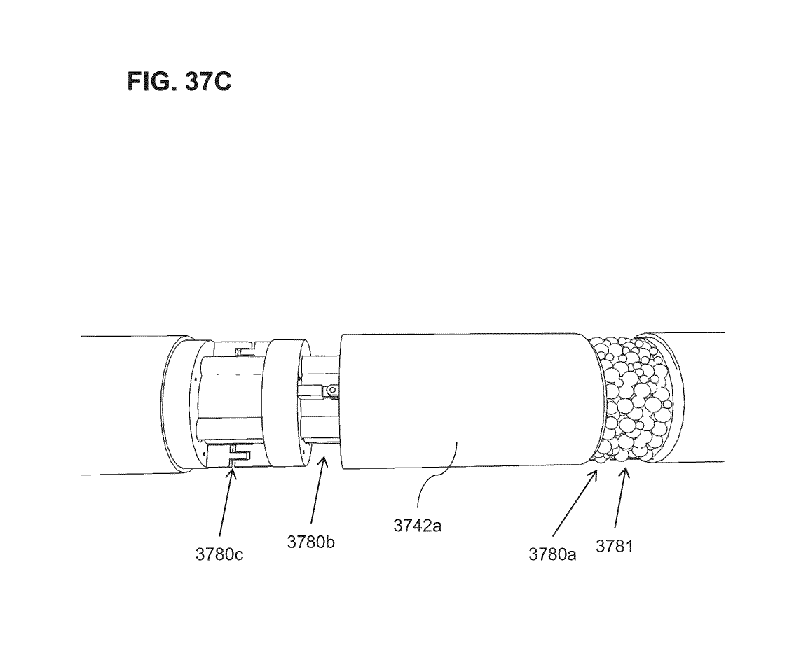

FIGS. 37A-C illustrate an endoscope with immobilizable joints in accordance with an embodiment of the current invention. For example an endoscope may be inserted into a desired location and/or configuration (for example a guide 3732 and/or a working channel 3728 may be inserted over a guidewire). The endoscope may optionally then be immobilized in place. Optionally the guidewire may then be removed with the endoscope remaining in place. Optionally the immobile endoscope may be used for access to the location and/or as a stable platform for tools in the location. Optionally the immobilization may be reversible. For example, after use, the endoscope may be remobilized and/or removed from the patient.

In the exemplary embodiment of FIGS. 37A-C, joints 3780a-c are reversibly immobilized by a collection of different sized balls 3781. Balls 3781 are optionally packed between frame elements (for example element 3782) and/or around joints 3780a-c and/or are sandwiched between two sleeves 3742a, 3742b. Optionally, when balls 3781 can move freely, then as joints 3780a-c move, balls 3781 are redistributed. Optionally, when the balls 3781 are immobilized, joint 3780a-c are optionally also immobilized. For example, a vacuum may be applied to the area where the balls 3781 are located collapsing the space between sleeves 3742a, 3742b and/or immobilizing balls 3781 and/or joints 3780a-c. When air is allowed to enter between sleeves 3742a, 3742b then balls 3781 and/or joints 3780a-c are remobilized.

FIG. 37A illustrates balls 3781 packed between two sleeves 3742a, 3742b in accordance with an embodiment of the current invention.

FIG. 37B illustrates balls 3781 packed around a frame element 3782 in accordance with an embodiment of the current invention.

FIG. 37C is a cutaway illustration of balls 3781 packed around joints 3780a-c in accordance with an embodiment of the current invention. In FIG. 37C, outer sleeve 3742a is cut away around joints 3780a-c to illustrate the immobilizing system. Joint 3780a is illustrated with balls 3781 in place. Joints 3780b, 3780c are illustrated with balls 3781 removed in order to better illustrate the joint.

### Closed Channels

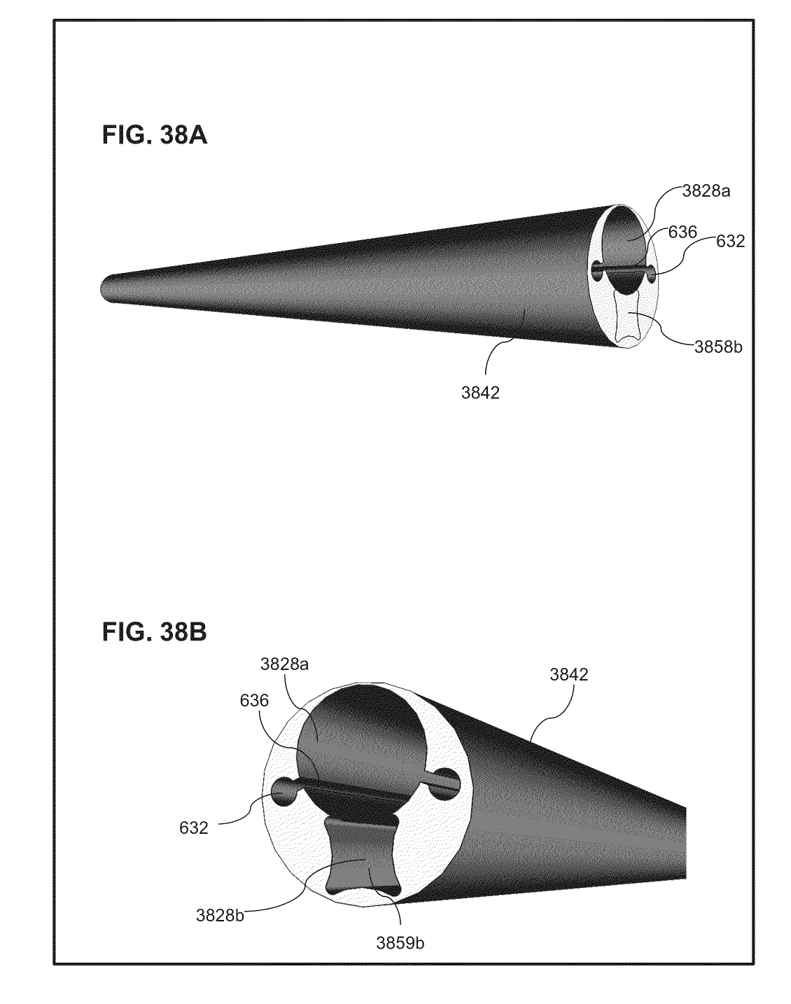

FIG. 38A is a perspective distal view of a sleeve with a closed channel in accordance with an embodiment of the present invention. In some embodiments, a sleeve 3842 may include a channel closed at a distal end. For example, a seal 3858b may seal a channel (for example channel 3828b illustrated in FIG. 38B) from exposure to the patient.

In some embodiments, the sealed 3858b may close a distal end of channel 3828b. Alternatively or additionally, seal 3858b may seal a portion of the length of the channel 3828b. For example, closed channel 3828b may be sealed along its length from exposure to other channels, for example a working channel 3828a and/or a guide channel 632.

FIG. 38B is a perspective proximal view of a sleeve with a closed channel in accordance with an embodiment of the present invention. In some embodiment sleeve 3842 may be single use. Alternatively or additionally sleeve 3842 may be cleaned and reused. In some embodiments, channel 3828b and/or a portion thereof may be permanently sealed. Alternatively or additionally, channel 3828b may be openable. For example channel 3828b may opened for cleaning and/or sterilizing. For example, seal 3858b may include a removable plug and/or a removable inner sleeve. Optionally the plug and/or inner sleeve may be single use. Optionally, the inner sleeve may be placed and/or mounted on a tool. For example tool and/or sleeve may be inserted and/or retrieved from channel 3828b together. Alternatively or additionally, a sleeve or plug may remain in the channel while a tool is inserted and/or removed.

In some embodiments sealed channel 3828b may protect a tool from exposure to a patient. For example, a tool that is difficult to clean may be inserted into a patient in a sealed channel. For example, a steering mechanism and/or a camera may be inserted into a sealed channel.

### Steering Mechanism

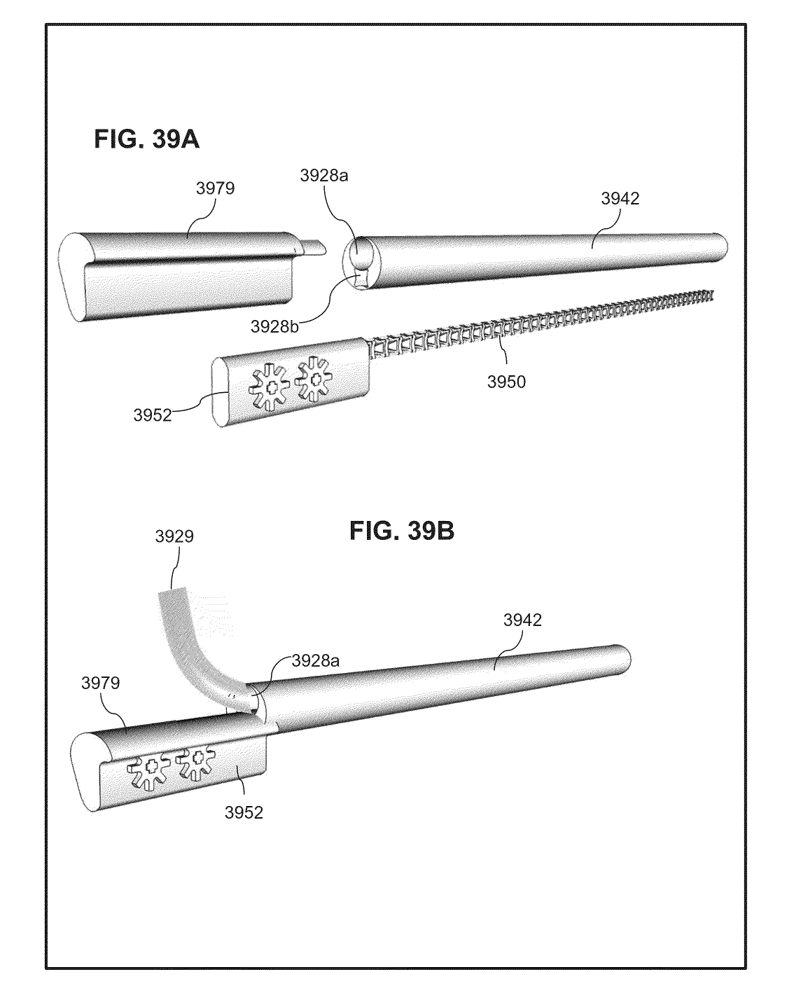

FIGS. 39A-B are a perspective proximal views of a sleeve and removable steering mechanism in accordance with an embodiment of the present invention. In some embodiments a steering mechanism 3952 endoprobe 3950 may be inserted into a closed channel 3928b and/or operatively connected to a sleeve 3942. For example channel 3928b may be sealed from a body of a patient and/or other channels of sleeve 3942. Optionally channel 3928b and/or endoprobe 3950 may be engagable, for example to prevent rotation of endoprobe 3950 in sleeve 3928b. In some embodiments, a distal portion of endoprobe 3950 may direct movement of a distal portion of sleeve 3942, for example for navigated inside a patient. Optionally, inside closed channel, endoprobe 3950 is protected from contamination and/or from contaminating a patient. Optionally when sleeve 3942 is in place in a patient, endoprobe 3950 may be removed from channel 3928b and/or channel 3928b can be used for another tool and/or function. Optionally, endoprobe 3950 is removed from sleeve 3942 before cleaning and/or sterilizing a reusable sleeve 3950 and/or before discarding a single use sleeve.

In some embodiments, a sealed channel (for example channel 3928b) and/or an steering mechanism endoscope (for example endoscope 3950) may extend for a distance ranging between 1% to 50% of the length of a sleeve in which it is formed and/or inserted (for example sleeve 3942) and/or between 50 to 80% and/or between 80 to 90% and/or between 90 to 98% and/or between 98% to 100% the length of the sleeve in which it is formed and/or inserted.

In some embodiments a sealed channel may be used with various tools. For example, multiple tools may be inserted into a sealed channel simultaneously and/or serially. For example, channel 3928b may include a window (for example in a distal seal thereof and/or along a side wall thereof. Optionally, an illumination source and/or an imaging sensor (for example a camera) may be placed in the channel. Alternatively or additionally, a tool may include a radiation source and/or a transceiver. Generally, a sealed channel may reduce the need for cleaning and/or sterilization of a tool, for example of a complex instrument.

FIG. 39A illustrates a proximal view of sleeve 3942, endoprobe 3950 and a shield 3979 in accordance with an embodiment of the current invention. In some embodiments, a sealed channel may have a cross section that does not have rotational symmetry (for example is not circular). For example, all or part of channel 3928b and/or endoprobe 3950 may have an irregular approximately rectangular cross section. For example, a the non-circular cross sections of channel 3928b and/or all or part of endoprobe 3950 may be match such that endoprobe 3950 is inhibited from and/or prevented from rotating within channel 3928b. Alternatively or additionally, endoprobe 3950 may engage a connector of channel 3928b, for example to fix a relative position and/or orientation of endoprobe 3950 and/or sleeve 3942.

In some embodiments, a sleeve may not include channel open on both ends and/or a sleeve may include a simple working channel open on both ends and/or may include multiple working channels and/or one or more sealed channels. Alternatively or additionally, a sleeve may include a guide channel and/or a further working channel.

In some embodiments, for example, the steering system may bend a distal portion of an endoscope between up to 30 degrees and/or up to 60 degrees and/or up to 90 degrees and/or up to 135 degrees and/or up to 180 degree and/or up to 279 degrees and/or a up to 360 degrees. The diameter and/or width of the insertable portion of an endoprobe steering system may range for example between 0 to 1 mm and/or between 1 to 2 mm and/or between 2 to 4 mm and/or between 4 to 8 mm and/or between 8 to 26 mm and/or between 18 to 36 mm. For example, an endoprobe steering system may have a length ranging between 1 to 30 mm and/or between 30 mm to 100 mm and/or between 100 mm to 300 mm and/or between 300 mm to 1 m and/or between 1 m to 2 m. The diameter and/or width of the closed channel may range for example between 0 to 1 mm and/or between 1 to 2 mm and/or between 2 to 4 mm and/or between 4 to 8 mm and/or between 8 to 26 mm and/or between 18 to 36 mm. For example, a closed channel may have a length ranging between 1 to 30 mm and/or between 30 mm to 100 mm and/or between 100 mm to 300 mm and/or between 300 mm to 1 m and/or between 1 m to 2 m.

FIG. 39B illustrates a proximal view of sleeve 3942 with endoprobe 3950 inserted therein and shield 3979 connected to steering system 3952 in accordance with an embodiment of the current invention. In some embodiments, a shield may protect and/or shield steering mechanism 3952 from contamination. For example, contamination may be prevented from fluids that pass through a working channel 3928a of the catheter. For example a shield 3979 may connect to a proximal portion of sleeve to steering mechanism 3952. Optionally, shield 3979 fits to the sleeve 3942. For example, shield may fit to sleeve 3942 in a determined orientation and/or determine an orientation of steering mechanism 3952 to sleeve 3942. Alternatively or additionally, a steering system and/or endoprobe may be connected to a sleeve without a shield.

In some embodiments, an extension 3929 may extend from the proximal end of a channel. For example extension 3929 distances the proximal opening of working channel 3928a from a steering mechanism 3952. In some embodiments, distancing the opening of a channel from a steering apparatus may inhibit cross contamination. For example, extension 3929 may include an inner tube and/or an extension of sleeve 3942. In some embodiments, an extension may extend from a proximal opening of closed channel (for example channel 3928b).

In some embodiments a sleeve may include a closed channel (for example channel 3928b) with or without a channel open on both ends (for example channel 3928a).

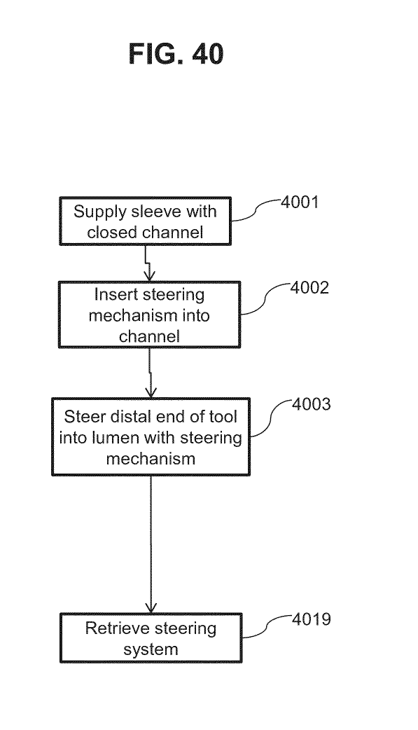

FIG. 40 is a flowchart illustration of a method of steering a sleeve in a lumen in accordance with an embodiment of the current invention. In some embodiments a sleeve is supplied 4001 for a catheter. The sleeve optionally includes a closed channel that is closed on a distal portion thereof. Optionally the closed channel includes a proximal opening. Optionally, a steering mechanism is inserted 4002 into the closed channel for example through the proximal opening thereof. In some embodiments, the steering mechanism is used to steer 4003 a distal portion of the sleeve through the lumen, for example to reach a region of interest. Optionally after the sleeve has reached its destination the steering mechanism is retrieved 4019 from the channel. For example, the channel may be used for another tool and/or the steering mechanism may be used in another sleeve.

### Manipulating a Tool

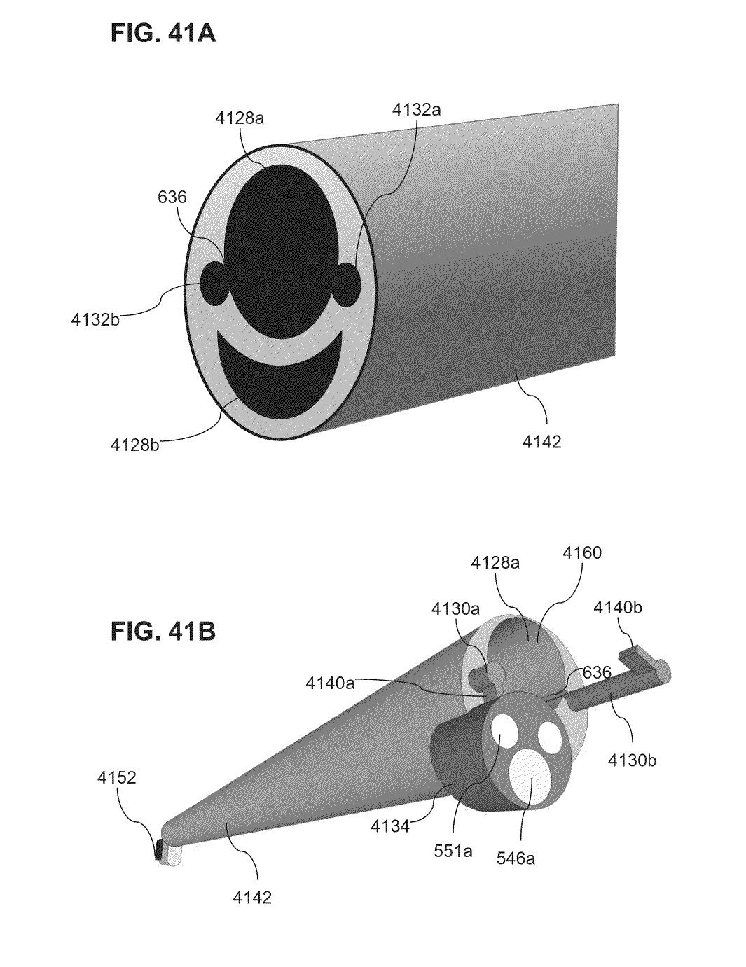

FIG. 41A is a perspective proximal view of a multi-channel sleeve in accordance with an embodiment of the present invention. In some embodiments a sleeve may include one or more sealed channels 4128b and/or one or more working channels 4128a and/or one or guides, for example guide channels 4132a and/or 4132b. Optionally, a channel 4128b may have a shape that fits a tool in a determined orientation. For example channel 4128b has a partial moon shape.

In some embodiments a steering mechanism 4152 may be inserted into a sealed channel 4128b (for example as illustrated in FIG. 41B). Optionally a carriage may be inserted into guide channel 4132a-b and/or one or more tools may pass through working channel 4128a into a patient. Optionally working channel 4128a has a distal opening 4160.

FIG. 41B is a perspective distal view of a sleeve with exemplary tools in accordance with an embodiment of the present invention. In some embodiments a user may insert one or more tools through a working channel 4128a into a lumen of a patient. Optionally the tools are connected to a distal portion of a carriage. For example, the tools may be manipulated may moving a proximal portion of the carriage.

In some embodiments, a carriage 4130a and/or 4130b may provide a direct connection between the distal and proximal portions of a sleeve 4142. For example, a carriage 4130a-b may be a continuous part. For example, a distal portion of carriage 4130a-b located outside the distal portion of the sleeve and/or an intermediate portion located inside sleeve 4142 and/or a proximal portion located outside a proximal opening of the sleeve may be connected directly to move together in at least one direction. For example, longitudinal movements of a proximal portion of carriage may cause the same longitudinal movement of an intermediate and/or distal portion. Optionally carriage 4130a-b may be elastic. For example some relative movement of the proximal and/or intermediate and/or distal portions may occur in one or more directions, but they may tend to return to a determined relative position. For example the positions of the proximal, intermediate and/or distal portions may be determined by a fixed stress strain relationship. Optionally, the stress strain curve may be continuous along the length of the carriage. For example, twisting of a proximal portion of carriage around its longitudinal axis may cause the same and/or proportional twisting movement of an intermediate and/or distal portion. Optionally carriages 4130a-b are flexible. For example, carriage 4130a-b may bend along with sleeve 4142 to fit along a curved body lumen. Optionally, the carriage is not articulated between the proximal and/or intermediate and/or distal portions thereof. For example carriage my not include a hinge and/or a non-elastic connection.

Optionally, a carriage (for example a distal portion thereof) is directly and/or rigidly and/or elastically connected to a mount. For example carriage 4130a is connected to mount 4140a and/or carriage 4130b is connected to mount 4140b. Optionally a tool 4134 (for example including a camera 546a and/or a light source 551a and/or a sensor and/or a pusher and/or a cutter and/or a pincer etc.) is connected to a mount (for example mount 4140a-b). A connection between a tool and a mount may optionally be permanent and/or reversible. Optionally, tool 4134 is illustrated supported by a mount to an outer surface of sleeve 4142. The tool is optionally connected through rigid and/or elastic connections to the distal portion of the carriage (for example outside the distal portion of the sleeve and/or inside the a lumen of a patient) and/or to the intermediate portion of the carriage (for example inside the sleeve) and/or to the proximal portion of the carriage (for example outside the proximal portion of the sleeve and/or outside the patient and/or handled by the user).

In some embodiments, by moving the proximal portion of the carriage with respect to the sleeve a user moves the distal portion of the carriage and/or the tool in the same way, similarly and/or proportionally with respect to the sleeve. For example a force in one or more directions (for example longitudinal and/or twisting) with respect to the sleeve on the proximal portion of the carriage is passed directly as a force with respect to the distal portion of the sleeve to the tool on the distal portion of the carriage (the force may be the same and/or slightly less for example due to friction losses). For example a force in one or more directions (for example longitudinal and/or twisting) on one carriage with respect to another carriage is on the proximal portion thereof is passed directly as a relative force with respect to tools on the distal portions of the carriages (the force may be the same and/or slightly less for example due to friction losses). By moving proximal portions of one or more carriages and/or tools with respect to each other and/or with respect to the catheter a user may move and/or manipulate the tools in a lumen of a patient. For example as described above, moving a pusher with relation to a pincer may open and/or close the pincer and/or a blade may be moved to cut and/or takes samples etc. Optionally a tools and/or a carriage may move as a unit. For example a tool may be moved and/or manipulated inside a patient by a user outside the patient without requiring separate controls and/or hinges and/or actuators and/or articulated elements in contact with the patient, inside the catheter and/or inside the patient. For example, multiple tools can be moved in and/or out of a patient and/or manipulated inside the patient serially through a single working channel and/or more than one working channel.

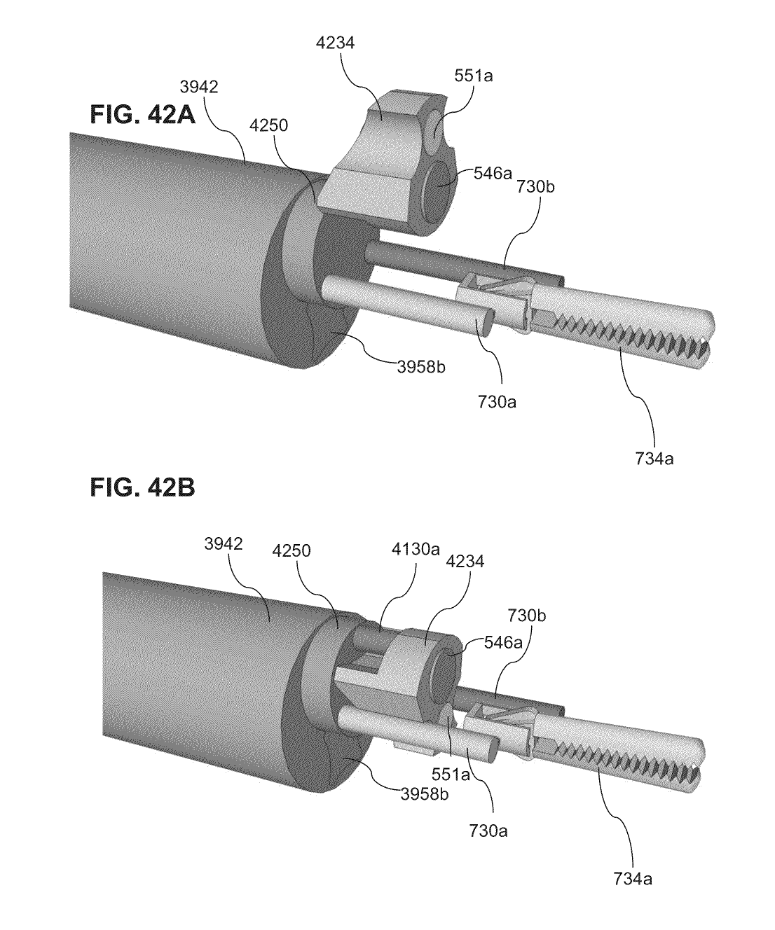

FIGS. 42A-B illustrate a multi-tool endoscope 4250 inserted through sleeve 3942 in accordance with an embodiment of the current invention. Optionally a tool may be designed to make space for other tools in an endoscope. For example, camera tool 4234 is designed with indentations for carriages 730a and 730b. For example to deploy camera 4234, pincer tool 734a may be moved out of the way (from example by twisting carriage 730a to move tool 734a downward away from camera tool 4234). Optionally then tool 4234 is moved distally ahead of the other tools and deployed upward by twisting carriage 4130a.

### Detection and Identification

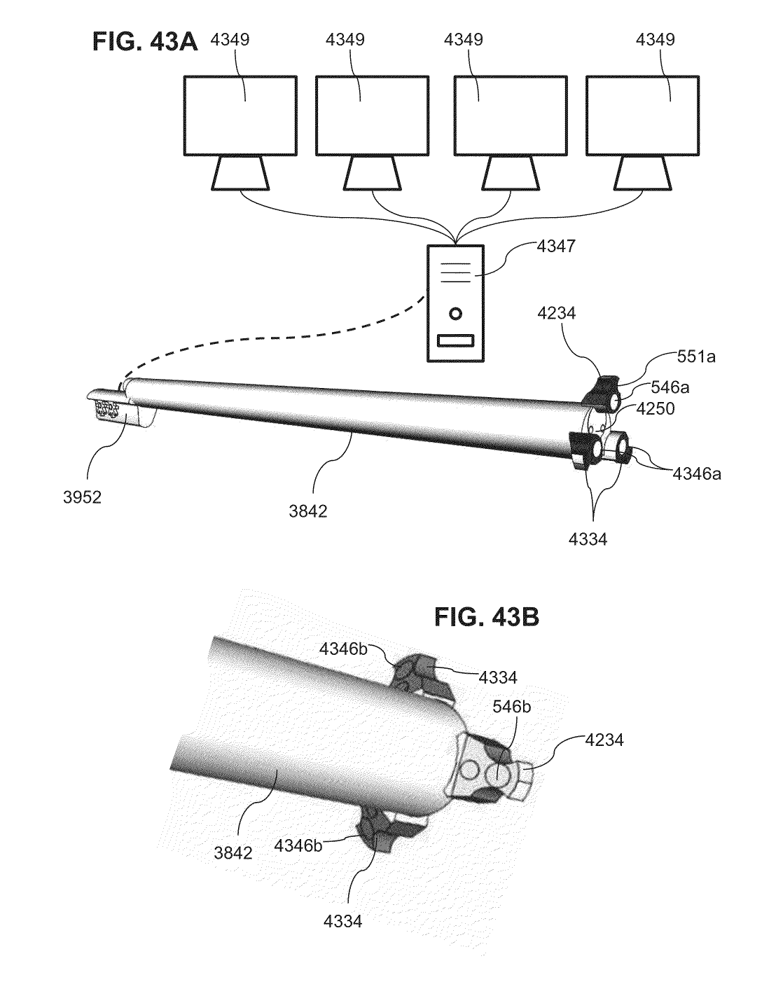

FIGS. 43A-B are a perspective view illustrations of a multi-camera endoscope in accordance with an embodiment of the current invention. In some embodiments there may be multiple tools 4234, 4334 including imaging sensors (for example forward facing camera 546a and/or cameras 4346a and/or rearward facing camera 546b and/or cameras 4346b). The output of the sensors may be processed by a processor 4347. The output of the cameras may be displayed on one or more output device 4349. For example output of one camera may be displayed on one output device and/or output of another camera may be displayed on a separate output device. Alternatively or additionally processor 4347 may process data and/or display one kind of data on one output device and another king of data on another device.

In some embodiments, recognizes a significant object, for example, an object that is suspected of being symptoms of a disease and/or a disease precursor. Optionally, processor 4347 displays on one output device 4349 data that is used for steering the sleeve. For example data for steering the sleeve may include an image from a single forward looking camera 546a and/or a collage of images from multiple forward directed cameras 546a, 4346a and/or other ancillary information. The steering data may be displayed on a first display device.

In some embodiments processor 4347 displays device information about significant objects on an output. For example, processor 4347 may include software for recognizing suspicious objects in images and/or collecting data on those objects. For example a catheter may include sensors for non-image data. For example, as the distal end of sleeve 3842 including tools 4234 and 4334 approaches and/or passes an object processor 4347 collects data from various sensors and/or determines if the object is a significant object. For an identified significant object, processor 4347 optionally collects up images of the object (for example different images may be at different angles and/or magnifications and/or different wave lengths) and/or non-image data on the object. Optionally processor 4347 displays data on the object on a second display device (and/or on separate window on the first display device). For example, the displayed data may include various images of the object and/or an estimated size and/or temperature of an object. The images and/or non-image data may be used by the processor to determine whether the object is significant and/or may be displayed to a user. Alternatively or additionally an indicator may be displayed on the first display to notify the user of the detection of the significant object. For example, a separate display device and/or window may be used for each significant object. Alternatively or additionally, processor 4347 may display in one display and/or window all objects of a particular type (for example all suspected polyps) while displaying objects of a second type (for example suspected infection sites) on separate display device. The user may optionally choose to use various tools to explore further some objects.

In some embodiments a catheter may include one image sensors for navigation and/or one or more image sensors for detection and/or identification of objects. For example, one forward facing sensor may be used for steering and/or detection of objects. Optionally one or more forwards and/or rearward sensors may optionally be used for detection and/or further identification.

In some embodiments, some sensors may be included on a multi-tool endoscope (for example tool 4234 on endoscope 4250) going through a working channel 3828a of a sleeve 3842. Other tools (for example tools 4334) are optionally directly placed into a region of interest through working channel 3828a using guides 632. A sensor (for example 4246a, 4246b, 4346a and/or 4346b) optionally sends output data to processor 4347 over a wire and/or a wireless channel. A processor 4347 optionally sends data to displays 4349 over a wire and/or a wireless channel.

It is expected that during the life of a patent maturing from this application many relevant technologies will be developed and the scope of the terms are intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A catheter for transporting material between outside a living creature and a region inside the living creature comprising:
a sleeve including:
a working channel including a proximal opening for access from outside of the living creature and a distal opening for positioning inside the region of the living creature;
a plurality of elongated guides extending along at least a portion of a length of said working channel, each of said guides in communication with said working channel along at least 5 cm of a length thereof;
a tool transportable between a first location outside said proximal opening, a second location inside said working channel and a third location outside said distal opening; and
a carriage mating to at least one guide of said plurality of elongated guides; said carriage
connected to said tool off center of a cross section of said tool,
traveling along said at least one guide from a proximal position to a distal position thereby conveying said tool from said second location to said third location, and
rotating around an axis of said at least one guide in said distal position thereby moving said tool to a fourth location away from an axis of said working channel.

2. The catheter of claim 1, wherein said tool includes a camera and in said fourth location a lens of said camera clears an outer cross section of the catheter.

3. The catheter of claim 1, further including a mount external to said working channel, said mount supporting said tool outside said working channel.

4. The catheter of claim 1, further including a longitudinal opening having a length of at least half a length of said working channel, said opening communicating between said working channel and the region.

5. The catheter of claim 4, further including a stiffener holding said longitudinal opening open.

6. A catheter for transporting material between outside a living creature and a region inside the living creature comprising:
a sleeve including a working channel including a proximal opening for access from outside of the living creature and a distal opening for positioning inside a region of the living creature said working channel configured for transporting a tool between a first location outside said proximal opening, a second location inside said working channel and a third location outside said distal opening; and
a mount external to said working channel, said mount supporting said tool on a portion of said sleeve outside said working channel in said third location.

7. The catheter of claim 6, wherein said mount supports said tool substantially out of a contour of a cross section of said working channel.

8. A method for performing one or more tasks inside a region of a living creature, said region accessible by a path comprising:
providing one or more elongated guides located along at least a portion of a length of the path, each of said one or more guides in communication with said path along said at least a portion of said length a sleeve including a channel having a proximal opening and closed on a distal end thereof;
inserting a first tool to a location on said path;
inserting a steering mechanism into said proximal opening;
introducing a distal portion of said sleeve into said region while steering said distal portion with said steering mechanism;
blocking at least 50% of a cross section of said path at said location with said first tool; and
unblocking said path by conveying said first tool along a first guide of said one or more guides to a site in said region retrieving said steering mechanism through said proximal opening.

9. A method for performing one or more tasks inside a region of a living creature, said region accessible through a working channel of a catheter said working channel including a distal opening inside said region and proximal opening outside said region comprising:
providing one or more elongated guides located along a length of the working channel, each of said one or more guides in communication with the working channel along said length each of said one or more guides having a proximal opening and a distal opening and providing first carriage connected to said tool off center of a cross section of said tool, said carriage mating to a first guide of said elongated guides said first carriage having a length greater than said working channel;
blocking at least 50% of a cross section of said working channel at a location with said first tool;
conveying said first tool along said working channel and out said distal opening of said working channel by means of said guide; and
unblocking said working channel by twisting a proximal end of said guide to move said tool away from an axis of said working channel.

10. The method of claim 9, further including:
supporting said tool inside said region on an outer surface of said catheter away from said working channel.

11. The method of claim 9, wherein said working channel and a closed channel with a proximal opening are included in a sleeve, the and the method further comprising:
inserting a steering mechanism into said proximal opening;
introducing a distal portion of said sleeve into said region while steering said distal portion with said steering mechanism; and
retrieving said steering mechanism through said proximal opening.

12. The catheter of claim 6, wherein in said tool includes a lens and at said third location said lens clears an outer cross section of the catheter.

13. The catheter of claim 6, further comprising:
a closed channel of length at least 50% of the sleeve, said closed channel having a proximal opening for access from outside of the living creature and said closed channel closed from said region;
a steering mechanism said steering mechanism reversibly inserted into said closed channel for steering said catheter.

14. The catheter of claim 13, further comprising:
an engagable fitting for inhibiting rotation of said steering mechanism with respect to said closed channel.

15. The catheter of claim 13, wherein said channel and said steering mechanism have matching non-rotationally symmetric cross sections such that the steering mechanism is inhibited from rotating in said channel.

16. The catheter of claim 15, wherein said steering mechanism fits into said channel in a single orientation.

17. The catheter of claim 16, wherein said closed channel is sealed off from said region.

18. The catheter of claim 13, further comprising:
a working channel including a proximal opening for access from outside of the living creature and a distal opening for positioning inside a region of the living creature said working channel for transporting a tool between a first location outside said proximal opening, a second location inside said working channel and a third location outside said distal opening.

19. The catheter of claim 18, wherein said closed channel is sealed off from said working channel.

20. The catheter of claim 1, further comprising:
a closed channel having a proximal opening for access from outside of the living creature and closed from said region;
a steering mechanism said steering mechanism reversibly inserted into said closed channel for steering said catheter.
